(19) United States
(12) Patent Application Publication
(10) Pub. No.: US 2024/0392009 A1
(43) Pub. Date: Nov. 28, 2024

(54) PHARMACEUTICAL COMPOSITIONS FOR ANTIBODIES AND MAKING AND USING THE SAME

(71) Applicant: Invetx, Inc., Boston, MA (US)

(72) Inventors: Krishnan Sampathkumar, Boston, MA (US); Chi-Ting Huang, Boston, MA (US)

(21) Appl. No.: 18/671,670

(22) Filed: May 22, 2024

Related U.S. Application Data
(60) Provisional application No. 63/503,659, filed on May 22, 2023.

(51) Int. Cl. C07K 16/28; A61K 9/08; A61K 47/10; A61K 47/18; A61K 47/22; A61K 47/26

(52) U.S. Cl. CPC C07K 16/283; A61K 9/08; A61K 47/10; A61K 47/183; A61K 47/22; A61K 47/26; C07K 2317/20

(57) ABSTRACT

A stable pharmaceutical formulation comprising: an antibody comprising a polypeptide having a canine CH2, CH3, IgG Fc region variant, or a canine FcRn-binding region thereof; a buffering agent at a pH in the range of from 4.5 to 7.0; a tonicity and/or stabilizing agent; a chelating agent; and a surfactant; wherein the formulation retains stability for up to twenty-four months in solution and is suitable for oral, rectal, transmucosal, intestinal, or parenteral administration.

### REFERENCE TO RELATED APPLICATIONS

This application claims benefit to and priority to U.S. Provisional Application Ser. No. 63/503,659, filed on May 22, 2023, which is hereby incorporated by this reference in its entirety.

### FIELD

This invention relates generally to stable pharmaceutical formulations of a pharmaceutically active antibody or fragments or domains of antibodies including bispecifics, tripecifics, and the like, comprising a polypeptide having canine or feline Complementarity-determining regions (CDRs) of a variable antibody domain, CH2, CH3, IgG Fc region variant, or a canine or feline FcRn-binding region thereof or a mixture of such antibody molecules for administration to a subject in need thereof.

### BACKGROUND

Currently, many antibodies are provided as lyophilized or freeze dried formulations in order to maintain stability through a product shelf-life of at least 2 years. Lyophilization or freeze-drying is a process which aids by removing or reducing water in the formulation that otherwise facilitates physical and chemical reactions of the polypeptide chain causing instability on long term storage. Lyophilized formulations of antibodies have a number of limitations, including a prolonged process for lyophilization and resulting high cost for manufacturing. In addition, a lyophilized formulation has to be reconstituted aseptically and accurately by practitioners prior to administering to the subject.

Antibodies are also provided in liquid formulations at concentrations comparable to or higher than the reconstituted lyophilized formulations so that there is no need to reconstitute the formulation prior to administration. This allows users and practitioners to more quickly and easily administer antibodies to a subject. However, liquid antibody preparations have short shelf lives and the antibodies may lose biological activity as a result of chemical and physical instabilities during the storage. Factors causing chemical instability include, but are not limited to deamidation, racemization, hydrolysis, oxidation, beta elimination or disulfide exchange, and the like. Factors causing physical instability include, but are not limited to antibody denaturation, aggregation, particle formation, precipitation, adsorption and the like. Among those factors, aggregation, deamidation and oxidation are known to be the most common causes of the antibody degradation (Wang et al., 1988, J. of Parenteral Science & Technology 42(Suppl) S4-S26; Cleland et al., 1993, Critical Reviews in Therapeutic Drug Carrier Systems 10(4): 307-377).

For a protein, and in particular, an antibody, to remain biologically active, a formulation must preserve intact the conformational integrity of at least a core sequence of the protein's amino acids while at the same time protecting the protein's multiple functional groups from degradation. Degradation pathways for proteins can involve chemical instability (i.e., any process which involves modification of the protein by bond formation or cleavage resulting in a new chemical entity) or physical instability (i.e., changes in the higher order structure of the protein). For a general review of stability of protein pharmaceuticals and factors affecting stability, see, for example, Manning, et al. (1989) Pharmaceutical Research 6:903-918 (the contents of which is incorporated by reference herein it its entirety). In addition, it is desirable to maintain stability when carrier polypeptides are not included in the formulation.

While the possible occurrence of protein instabilities is widely appreciated, it is impossible to predict particular instability issues for a particular protein. Any of these instabilities, or a combination thereof, can potentially result in the formation of a polypeptide by-product or derivative having undesirable effects such as lowered activity or efficacy, increased toxicity, and/or increased immunogenicity and the like. Indeed, polypeptide precipitation can lead to thrombosis and/or non-homogeneity of dosage form, and particle formation can cause undesirable immune reactions in the subject receiving the polypeptide. Thus, the safety, efficacy and immunogenicity of any pharmaceutical formulation of a polypeptide is directly related to its stability.

Accordingly, there continues to exist a need for formulations that not only maintain the stability and biological activity of biological polypeptides upon storage and delivery, but also are suitable for various routes of therapeutic administration.

Thus, provided herein are antibody formulation compositions suitable for presentation in single dose or multi-dose vials or syringes that provide acceptable product quality and stability for over 24 months of shelf life in the liquid state.

### SUMMARY

In accordance with the purpose(s) of this invention, as embodied and broadly described herein, this invention, in one aspect, relates to a stable pharmaceutical formulation comprising: (a) 5 to 100 mg/ml of an antibody or fragments or domains of antibodies comprising a polypeptide having a canine or feline CDR of variable antibody domain, CH2, CH3, IgG Fc region variant, or a canine or feline FcRn-binding region thereof, (b) 5 to 50 mM of a buffering agent at a pH in the range of from 4.0 to 7.2; (c) 0.01 to 35% (w/v) of a tonicity and/or stabilizing agent; and (d) 0.002 to 10% (w/v) of a surfactant; wherein the formulation is a single-dose or multi-dose formulation, and wherein the formulation retains stability for up to twenty-four months in solution.

In a particular embodiment, the formulation further comprises 0.01 to 5.0 mM of a chelating agent. In another embodiment, the multi-dose formulation further comprises an antimicrobial preservative selected from a group comprising benzyl alcohol, phenol, m-cresol, benzalkonium chloride, benzalthonium chloride, phenoxyethanol and methyl paraben or mixtures thereof at about a concentration of 0.1 to 1.5% (w.v). In particular embodiments, the antimicrobial preservative is benzyl alcohol.

In one embodiment, the buffering agent is selected from a group consisting of succinate, histidine, histidine hydrochloride (HCl), phosphate, Tris, diethanolamine, citrate, acetate, other organic acids and mixtures thereof. The buffering agent may preferably be acetate, histidine, or histidine HCl. In another embodiment, the buffering agent is present at a concentration of 5 to 50 mM and maintains a physiologically suitable pH in the range of from pH 4.0 to pH 7.2.

In another embodiment, the tonicity and/or stabilizing agent is selected from a group consisting of CaCl₂, NaCl, MgCl₂, lactose, sorbitol, sucrose, mannitol, trehalose, raffinose, polyethylene glycol, hydroxyethyl starch, cyclodextrin, glycine or other stabilizing amino acids including proline, arginine, glutamine, and the like and mixtures thereof. In an alternate embodiment, the tonicity and/or stabilizing agent is sucrose, trehalose, or sorbitol. In one embodiment, the tonicity and/or stabilizing agent is present at a concentration 0.01 to 35% (w/v) while in an alternate embodiment the tonicity and/or stabilizing agent may be present at 0.01% to 15% w/v.

In one embodiment, the chelating agent is selected from a group consisting of aminopolycarboxylic acids, hydroxyaminocarboxylic acids, N-substituted glycines, 2-(2-amino-2-oxoethyl) aminoethane sulfonic acid (BES), deferoxamine (DEF), citric acid, niacinamide, desoxycholates, diethylenetriamine pentaacetic acid 5 (DTPA), nitrilotriacetic acid (NTA), N-2-acetamido-2-iminodiacetic acid (ADA), bis(aminoethyl)glycolether, N,N,N′,N′-tetraacetic acid (EGTA), trans-diaminocyclohexane tetraacetic acid (DCTA), glutamic acid, and aspartic acid, N-hydroxyethyliminodiacetic acid (HIMDA), N,N-bis-hydroxyethylglycine (bicine) and N-(trishydroxymethylmethyl) 10 glycine (tricine), glycylglycine, sodium desoxycholate, ethylenediamine, propylenediamine, diethylenetriamine, triethylenetetraamine (trien), ethylenediaminetetraacetic acid (EDTA), disodium EDTA, calcium EDTA oxalic acid, malate, citric acid, citric acid monohydrate, and trisodium citrate-dihydrate, 8-hydroxyquinolate, amino acids, histidine, cysteine, methionine, peptides, polypeptides, and proteins and mixtures thereof. In another embodiment, the chelating agent is EDTA, disodium EDTA, or calcium EDTA. The chelating agent is present at a concentration of 0.01 to 5.0 mM. In an alternate embodiment of a formulation according to the present invention, the chelating agent is present from about 0.1 to about 2.0 mM

In yet another embodiment, the surfactant is selected from a group consisting of polysorbates, poloxamers, tritons, sodium dodecyl sulfate, sodium laurel sulfate, sodium octyl glycoside, lauryl-sulfobetaine, myristyl-sulfobetaine, linoleyl-sulfobetaine, stearyl-sulfobetaine, lauryl-sarcosine, myristyl-sarcosine, linoleyl-sarcosine, stearyl-sarcosine, linoleyl-betaine, myristyl-betaine, cetyl-betaine, lauroamidopropyl-betaine, cocamidopropyl-betaine, linoleamidopropyl-betaine, myristamidopropyl-betaine, palmidopropyl-betaine, isostearamidopropyl-betaine, myristamidopropyl-dimethylamine, palmidopropyl-dimethylamine, isostearamidopropyl-dimethylamine, sodium methyl cocoyl-taurate, disodium methyl oleyl-taurate, dihydroxypropyl PEG 5 linoleammonium chloride, polyethylene glycol, polypropylene glycol, and mixtures thereof. In another embodiment, the polysorbate is selected from the group consisting of polysorbate 20, polysorbate 21, polysorbate 40, polysorbate 60, polysorbate 61, polysorbate 65, polysorbate 80, polysorbate 81, polysorbate 85, and mixtures thereof. The surfactant is present at a concentration of 0.002 to 1.0% (w/v).

In still another embodiment, the pharmaceutical formulation further comprises an antioxidant selected from a group consisting of GLA (gamma-linolenic acid)-lipoic acid, DHA (docosahexaenoic acid)-lipoic acid, GLA-tocopherol, di-GLA-3,3′-thiodipropionic acid, DGLA (dihomo-gamma-linolenic acid), AA (arachidonic acid), SA (salicylic acid), EPA (eicosapentaenoic acid) or DHA (docosahexaenoic acid), phenolic anti-oxidants including, polyenes, unsaturated sterols, ascorbic acid, organosulfur compounds, terpenes and amino acid antioxidants. In one embodiment, the amino acid antioxidants are selected from a group consisting of methionine, cysteine, carnosine and analogs thereof. The antioxidant is present at a concentration of 0.02 mM to about 100 mM.

In still another embodiment, the pharmaceutical formulation further comprises a preservative selected from a group consisting of phenols, m-cresol, benzyl alcohol, benzalkonium chloride, benzalthonium chloride, phenoxyethanol and methyl paraben. The preservative is present at a concentration of about 0.001% w/v to about 10% w/v.

In still another embodiment, the pharmaceutical formulation is suitable for oral, rectal, transmucosal, intestinal, or parenteral administration. In another embodiment parenteral administration is selected from intravenous, intraarterial, intraperitoneal, intrathecal, intraventricular, intraurethral, intrasternal, intracranial, intramuscular, intra-ossial, intradermal or subcutaneous administration.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate an embodiment(s) of the invention and together with the description, serve to explain the principles of the invention.

FIG. 1 shows a DSC thermogram overlay of IVX-01101 pH/Buffer screening study evaluation.

FIGS. 2A-2C show the visual appearance of samples at different time points, from left to right the formulation No. is 01 to 06 (as shown in Table 26) (FIG. 2A) T0; (FIG. 2B) 40° C.-2 W; (FIG. 2C) 40° C.-4 W.

FIGS. 3A-3D show non-GMP stable pool production stability data at −70° C.±10° C. for the IVX-03023 monoclonal antibody produced in a 15 L bioreactor. FIG. 3A-3B show that drug substance was stable at recommended long term storage temperature of −70±−10° C. for 1 year and well above the acceptance criteria for all the product quality attributes including size heterogeneity % monomer (FIG. 3A), % purity (FIG. 3B), protein concentration (FIG. 3C), and charge heterogeneity (FIG. 3D).

FIGS. 4A-4H show non-GMP stable pool production stability data at 5° C.±3° C. for the IVX-03023 monoclonal antibody produced in a 15 L bioreactor. FIGS. 4A-4H show that drug product was stable at recommended long term storage temperature of 2-8° C. for 1 year well above the acceptance criteria for all the product quality attributes including size heterogeneity % monomer (FIG. 4A), size heterogeneity % HMW (FIG. 4B), purity (FIG. 4C), charge heterogeneity (FIG. 4D), protein concentration (FIG. 4E), ELISA potency (FIG. 4F), subvisible particle>10/mL (FIG. 4G) and subvisible particle>25/mL (FIG. 4H). Potency of the molecule is maintained.

FIGS. 5A-5E show non-GMP stable pool production stability data at 25° C.±2° C. for the IVX-03023 monoclonal antibody produced in a 15 L bioreactor. FIGS. 5A-5E show that drug product was stable at recommended long term storage temperature of 25° C. for 3 months well above the acceptance criteria for all the product quality attributes including size heterogeneity % monomer (FIG. 5A), size heterogeneity % HMW (FIG. 5B), purity (FIG. 5C), charge heterogeneity (FIG. 5D), and protein concentration (FIG. 5E).

FIGS. 6A-6D show non-GMP stable pool production stability data at −70° C.±10° C. for the IVX-06076 monoclonal antibody produced in a 15 L bioreactor. FIG. 3A-3B show that drug substance was stable at recommended long term storage temperature of −70±−10° C. for 6 months and well above the acceptance criteria for all the product quality attributes including size heterogeneity % monomer (FIG. 6A), % purity (FIG. 6B), protein concentration (FIG. 6C), and charge heterogeneity (FIG. 6D).

FIGS. 7A-7H show non-GMP stable pool production stability data at 5° C.±3° C. for the IVX-06076 monoclonal antibody produced in a 15 L bioreactor. FIGS. 7A-7H show that drug product was stable at recommended long term storage temperature of 5° C. for 1 year well above the acceptance criteria for all the product quality attributes including size heterogeneity % monomer (FIG. 7A), size heterogeneity % HMW (FIG. 7B), purity (FIG. 7C), charge heterogeneity (FIG. 7D), protein concentration (FIG. 7E), ELISA potency (FIG. 7F), subvisible particle>10/mL (FIG. 7G) and subvisible particle>25/mL (FIG. 7H). Potency of the molecule is maintained.

FIGS. 8A-8H show non-GMP stable pool production stability data at 25° C.±2° C. for the IVX-06076 monoclonal antibody produced in a 15 L bioreactor. FIGS. 8A-8H show that drug product was stable at recommended long term storage temperature of 25° C. for 6 months well above the acceptance criteria for all the product quality attributes including size heterogeneity % monomer (FIG. 8A), size heterogeneity % HMW (FIG. 8B), purity (FIG. 8C-8D), protein concentration (FIG. 8E), binding ELISA (FIG. 8F), subvisible particle>10/mL (FIG. 8G) and subvisible particle>25/mL (FIG. 8H).

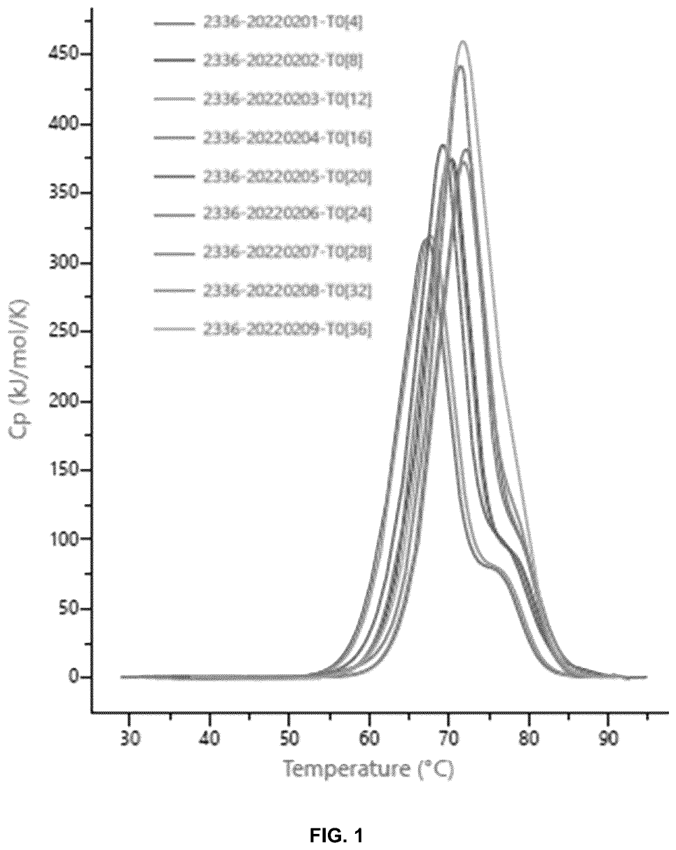

### DETAILED DESCRIPTION

The present invention may be understood more readily by reference to the following detailed description of preferred embodiments of the invention and the Examples included therein and to the Figures and their previous and following description.

### I. Definitions

To facilitate an understanding of the principles and features of the various embodiments of the disclosure, various illustrative embodiments are explained herein. Although exemplary embodiments of the disclosure are explained in detail, it is to be understood that other embodiments are contemplated. Accordingly, it is not intended that the disclosure is limited in its scope to the details of construction and arrangement of components set forth in the description or examples. The disclosure is capable of other embodiments and of being practiced or carried out in various ways.

In describing the exemplary embodiments, specific terminology will be resorted to for the sake of clarity. As used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value.

Similarly, as used herein, “substantially free” of something, or “substantially pure”, and like characterizations, can include both being “at least substantially free” of something, or “at least substantially pure”, and being “completely free” of something, or “completely pure.”

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

All formulations of antibodies and/or antibody fragments that specifically bind to an antigen of interest are herein collectively referred to as “formulations of the disclosure”, “liquid formulations of the disclosure”, “high concentration stable liquid formulations of the disclosure”, “antibody liquid formulations of the disclosure”, or “antibody formulations of the disclosure.”

As used herein, the terms “antibody” and “antibodies” (immunoglobulins) encompass monoclonal antibodies (including full-length monoclonal antibodies), polyclonal antibodies, multispecific antibodies (e.g., bispecific antibodies) formed from at least two intact antibodies, human antibodies, humanized antibodies, camelised antibodies, chimeric antibodies, single-chain Fvs (scFv), single-drain antibodies, single domain antibodies, domain antibodies, Fab fragments, F(ab′)2 fragments, antibody fragments that exhibit the desired biological activity, disulfide-linked Fvs (sdFv) and anti-idiotypic (anti-Id) antibodies (including, e.g., anti-Id antibodies to antibodies of the disclosure), intrabodies, and epitope-binding fragments of any of the above. In particular, antibodies include immunoglobulin molecules, biologically active fragments of the disclosed molecules and immunologically active fragments of immunoglobulin molecules, i.e., molecules that contain an antigen-binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass.

Native antibodies are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Light chains are classified as either lambda chains or kappa chains based on the amino acid sequence of the light chain constant region. The variable domain of a kappa light chain may also be denoted herein as VK. The term “variable region” may also be used to describe the variable domain of a heavy chain or light chain. Particular amino acid residues are believed to form an interface between the light and heavy chain variable domains. Such antibodies may lie derived from any mammal, including, but not limited to, humans, monkeys, pigs, horses, rabbits, dogs, cats, mice, etc.

The term “antibody fragment” refers to less than an intact antibody structure, including, without limitation, an isolated single antibody chain, an Fv construct, a Fab construct, an Fc construct, a light chain variable or complementarity determining region (CDR) sequence, and the like.

The term “variable” refers to the fact that certain portions of the variable domains differ extensively in sequence among antibodies and are responsible for the binding specificity of each particular antibody for its particular antigen. However, the variability is not evenly distributed through the variable domains of antibodies. It is concentrated in segments called Complementarity Determining Regions (CDRs) both in the light chain and the heavy chain variable domains. The more highly conserved portions of the variable domains are called the framework regions (FW). The variable domains of native heavy and light chains each comprise four FW regions, largely adopting a 3-sheet configuration, connected by three CDRs, which form loops connecting, and in some cases forming part of, the β-sheet structure. The CDRs in each chain are held together in close proximity by the FW regions and, with the CDRs from the other chain, contribute to the formation of the antigen-binding site of antibodies (see, Kabat et al., Sequences of Proteins of Immunological Interest, 5th Ed. Public Health Service. National Institutes of Health, Bethesda, Md. (1991)). The constant domains are generally not involved directly in antigen binding, but may influence antigen binding affinity and may exhibit various effector functions, such as participation of the antibody in ADCC, CDC, antibody-dependent phagocytosis and/or apoptosis.

The term “hypervariable region” when used herein refers to the amino acid residues of an antibody which are associated with its binding to antigen. The hypervariable regions encompass the amino acid residues of the “complementarity determining regions” or “CDRs” of the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” in the light chain variable domain and in the heavy chain van able domain; Chothia and Lesk, J. Mol. Biol. 196:901-917 (1987)). “Framework” or “FW” residues are those variable domain residues flanking the CDRs FW residues are present in chimeric, humanized, human, domain antibodies, diabodies, vaccibodies, linear antibodies, and bispecific antibodies.

As used herein “Fc region” includes the polypeptides comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cgamma2 and Cgamma3 (Cγ2 and Cγ3) and the hinge between Cgamma1 (Cγ1) and Cgamma2 (Cγ2). Although the boundaries of the Fc region may vary, the human IgG heavy chain Fc region is usually defined to comprise residues C226 or P230 to its carboxyl-terminus, wherein the numbering is according to the EU index as in Kabat et al. (1991, NIH Publication 91-3242, National Technical Information Service, Springfield. Va.). The “EU index as set forth in Kabat” refers to the residue numbering of the human IgG1 EU antibody as described in Kabat et al supra. Fc may refer to this region in isolation, or this region in the context of an antibody, antibody fragment, or Fc fusion protein. An Fc variant protein may be an antibody, Fc fusion, or any protein or protein domain that comprises an Fc region Particularly preferred are proteins comprising variant Fc regions, which are non-naturally occurring variants of an Fc region. The amino acid sequence of a non-naturally occurring Fc region (also referred to herein as a “variant Fc region”) comprises a substitution, insertion and/or deletion of at least one amino acid residue compared to the wild type amino acid sequence. Any new amino acid residue appearing in the sequence of a variant Fc region as a result of an insertion or substitution may be referred to as a non-naturally occurring amino acid residue. Note: Polymorphisms have been observed at a number of Fc positions, including but not limited to Kabat 270, 272, 312, 315, 356, and 358, and thus slight differences between the presented sequence and sequences in the prior art may exist.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, immunoglobulins can be assigned to different classes. Presently there are five major classes of immunoglobulins: IgA, IgD, IgE, IgG, and IgM, and several of these may be further divided into subclasses (isotypes), e.g., IgG1, IgG2, IgG3, IgG4, IgA, and IgA2 (as defined by mouse and human designation). The heavy-chain constant domains that correspond to the different classes of immunoglobulins are called alpha, delta, epsilon, gamma, and mu, respectively. The subunit structures and three-dimensional configurations of different classes of immunoglobulins are well known in multiple species. The prevalence of individual isotypes and functional activities associated with these constant domains are species-specific and must be experimentally defined.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, monoclonal antibodies are advantageous in that they can be synthesized by hybridoma cells that are uncontaminated by other immunoglobulin producing cells. Alternative production methods are known to those trained in the art, for example, a monoclonal antibody may be produced by cells stably or transiently transfected with the heavy and light chain genes encoding the monoclonal antibody.

The modifier “monoclonal” indicates the character of the antibody as being obtained from a substantially homogeneous population of antibodies, and is not to be construed as requiring engineering of the antibody by any particular method. The term “monoclonal” is used herein to refer to an antibody that is derived from a clonal population of cells, including any eukaryotic, prokaryotic, or phage clone, and not the method by which the antibody was engineered. For example, the monoclonal antibodies to be used in accordance with the present disclosure may be made by the hybridoma method first described by Kohler et al., Nature, 256:495 (1975), or may be made by any recombinant DNA method (see, e.g., U.S. Pat. No. 4,816,567), including isolation from phage antibody libraries using the techniques described in Clackson et al., Nature, 352:624-628 (1991) and Marks et al., J. Mol. Biol., 222.581-597 (1991), for example. These methods can be used to produce monoclonal mammalian, chimeric, humanized, human, domain antibodies, diabodies, vaccibodies, linear antibodies, and bispecific antibodies.

A “human antibody” can be an antibody derived from a human or an antibody obtained from a transgenic organism that has been “engineered” to produce specific human antibodies in response to antigenic challenge and can be produced by any method known in the art. In certain techniques, elements of the human heavy and light chain loci are introduced into strains of the organism derived from embryonic stem cell lines that contain targeted disruptions of the endogenous heavy chain and light chain loci. The transgenic organism can synthesize human antibodies specific for human antigens, and the organism can be used to produce human antibody-secreting hybridomas. A human antibody can also be an antibody wherein the heavy and light chains are encoded by a nucleotide sequence derived from one or more sources of human DNA. A fully human antibody also can be constructed by genetic or chromosomal transfection methods, as well as phage display technology, or in vitro activated ICOS expressing T cells, all of which are known in the art.

An antibody may also refer to a canine antibody or one that has been caninised. As used herein, the term “canine” includes all domestic dogs, Canis lupus familiaris or Canis familiaris, unless otherwise indicated. “Caninized” forms of non-canine (e.g., murine) antibodies are genetically engineered antibodies that contain minimal sequence derived from non-canine immunoglobulin. Caninized antibodies are canine immunoglobulin sequences (recipient antibody) in which hypervariable region residues of the recipient are replaced by hypervariable region residues from a non-canine species (donor antibody) such as mouse having the desired specificity, affinity, and capacity. In some instances, framework region (FR) residues of the canine immunoglobulin sequences are replaced by corresponding non-canine residues. Furthermore, caninized antibodies may include residues that are not found in the recipient antibody or in the donor antibody. These modifications are made to further refine antibody performance. In general, the caninized antibody will include substantially all of at least one, and typically two, variable domains, in which all or substantially all of the hypervariable regions correspond to those of a non-canine immunoglobulin sequence and all or substantially all of the FRs are those of a canine immunoglobulin sequence. The caninized antibody optionally also will comprise a complete, or at least a portion of an immunoglobulin constant region (Fc), typically that of a canine immunoglobulin sequence.

An antibody may also refer to a feline antibody or one that has been felinized. As used herein the term “feline” includes all members of the cat family and other members of Felidae, which includes the subfamilies Pantherinae and Felinae (conventionally designated a felid) as well as domestic cats (Felis catus). “Felinized” forms of non-feline (e.g., murine) antibodies are genetically engineered antibodies that contain minimal sequence derived from non-feline immunoglobulin. Felinized antibodies are feline immunoglobulin sequences (recipient antibody) in which hypervariable region residues of the recipient are replaced by hypervariable region residues from a non-feline species (donor antibody) such as mouse having the desired specificity, affinity, and capacity. In some instances, framework region (FR) residues of the feline immunoglobulin sequences are replaced by corresponding non-feline residues. Furthermore, felinized antibodies may include residues that are not found in the recipient antibody or in the donor antibody. These modifications are made to further refine antibody performance. In general, the felinized antibody will include substantially all of at least one, and typically two, variable domains, in which all or substantially all of the hypervariable regions correspond to those of a non-feline immunoglobulin sequence and all or substantially all of the FRs are those of a feline immunoglobulin sequence. The felinized antibody optionally also will comprise a complete, or at least a portion of an immunoglobulin constant region (Fc), typically that of a feline immunoglobulin sequence.

An antibody may also refer to an antibody from a production animal. As used herein the term “production animal antibody” includes antibodies from livestock including but not limited to cows, horses, sheep, goats, chickens, ostriches, geese, and the like.

The term “heterochimeric” as defined herein, refers to an antibody in which one of the antibody chains (heavy or light) is canonized or felinized while the other is chimeric.

The terms “Fc receptor” or “FcR” are used to describe a receptor that binds to the Fc region of an antibody. In one embodiment, the FcR is a native sequence human FcR. Moreover, in certain embodiments, the FcR is one which binds an IgG antibody (a gamma receptor) and includes receptors of the FcγRI, FcγRII, FcγRIII, and FcγRIV subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (See, Daëron. Annu. Rev. Immunol., 15:203-234 (1997)). FcRs are reviewed in Ravetch and Kinet, Annu. Rev. Immunol., 9:457-92 (1991): Capel et al., Immunomethods, 4:25-34 (i 994); and de Haas et al., J. Lab. Clin. Med., 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., Immunol., 117:587 (1976) and Kim et al., J. Immunol., 24: 249 (1994)).

“Affinity” of an antibody for an epitope to be used in the treatments described herein is a term well understood in the art and means the extent, or strength, of binding of antibody to epitope. Affinity may be measured and/or expressed in a number of ways known in the art, including, but not limited to, equilibrium dissociation constant (KD or Kd), apparent equilibrium dissociation constant (KD′ or Kd′), and IC50 (amount needed to effect 50% inhibition in a competition assay). It is understood dial, for purposes of this disclosure, an affinity is an average affinity for a given population of antibodies which bind to an epitope. Values of KD′ reported herein in terms of mg IgG per mL or mg/mL indicate mg Ig per mL of serum, although plasma can be used.

When antibody affinity is used as a basis for administration of the treatment methods described herein, or selection for the treatment methods described herein, antibody affinity can be measured before and/or during treatment, and the values obtained can be used by a clinician in assessing whether a human patient is an appropriate candidate for treatment.

As used herein, the term “avidity” is a measure of the overall binding strength (i.e., both antibody arms) with which an antibody binds an antigen. Antibody avidity can be determined by measuring the dissociation of the antigen-antibody bond in antigen excess using any means known in the art, such as, but not limited to, by the modification of indirect fluorescent antibody as described by Gray et al., J. Virol. Meth., 44:11-24 (1993)

An “epitope” is a term well understood in the art and means any chemical moiety that exhibits specific binding to an antibody. An “antigen” is a moiety or molecule that contains an epitope, and, as such, also specifically binds to antibody.

The term “antibody half-life” as used herein means a pharmacokinetic property of an antibody that is a measure of the mean survival time if antibody molecules following their administration. Antibody half-life can be expressed as the time required to eliminate 50 percent of a known quantity of immunoglobulin from the patient's body or a specific compartment thereof, for example, as measured in serum or plasma, i.e., circulating half-life, or in other tissues. Half-life may vary from one immunoglobulin or class of immunoglobulin to another. In general, an increase in antibody half-life results in an increase in mean residence time (MRT) in circulation for the antibody administered.

The term “isotype” refers to the classification of an antibody's heavy or light chain constant region. The constant domains of antibodies are not involved in binding to antigen, but exhibit various effector functions. Depending on the amino acid sequence of the heavy chain constant region, a given human antibody or immunoglobulin can be assigned to one of five major classes of immunoglobulins: IgA, IgD. IgE. IgG, and IgM. Several of these classes may be further divided into subclasses (isotypes), e.g., IgG1 (gamma 1), IgG2 (gamma 2), IgG3 (gamma 3), and IgG4 (gamma 4), and IgA1 and IgA2. The heavy chain constant regions that correspond to the different classes of immunoglobulins are called α, δ, ε, γ, and μ, respectively. The structures and three-dimensional configurations of different classes of immunoglobulins are well-known. Of the various human immunoglobulin classes, only human IgG1, IgG2, IgG3. IgG4, and IgM are known to activate complement. Human IgG1 and IgG3 are known to mediate ADCC in humans. Human light chain constant regions may be classified into two major classes, kappa and lambda.

As used herein, the term “immunogenicity” means that a compound is capable of provoking an immune response (stimulating production of specific antibodies and/or proliferation of specific T cells).

As used herein, the term “antigenicity” means that a compound is recognized by an antibody or may bind to an antibody and induce an immune response.

The term “excipient” as used herein refers to an inert substance which is commonly used as a diluent, vehicle, preservative, binder or stabilizing agent for drugs which imparts a beneficial physical property to a formulation, such as increased protein stability, increased protein solubility, and decreased viscosity. Examples of excipients include, but are not limited to proteins (for example, but not limited to serum albumin), amino acids (for example, but not limited to aspartic acid, glutamic acid, lysine, arginine, glycine), surfactants (for example, but not limited to SDS, Tween 20, Tween 80, polysorbate and nonionic surfactants), saccharides (for example but not limited to, glucose, sucrose, maltose and trehalose), polyols (for example, but not limited to mannitol and sorbitol), fatty acids and phospholipids (for example, but not limited to alkyl sulfonates and caprylate). For additional information regarding excipients, see Remington's Pharmaceutical Sciences (by Joseph P. Remington, 18^{th} ed., Mack Publishing Co., Easton. Pa.), which is incorporated herein in its entirety.

The phrase “pharmaceutically acceptable” as used herein means approved by a regulatory agency of the Federal or a state government, or listed in the U.S. Pharmacopeia, European Pharmacopia or other generally recognized pharmacopeia for use in animals, and more particularly in humans.

The terms “stability” and “stable” as used herein in the context of a liquid formulation comprising an antibody (including antibody fragment thereof) that specifically binds to an antigen of interest and refers to the resistance of the antibody (including antibody fragment thereof) in the formulation to aggregation, degradation or fragmentation under given manufacture, preparation, transportation and storage conditions. The “stable” formulations of the disclosure retain biological activity under given manufacture, preparation, transportation and storage conditions. The stability of said antibody (including antibody fragment thereof) can be assessed by degrees of aggregation, degradation or fragmentation, as measured by SEC (size exclusion chromatography which includes SE-HPLC (size exclusion high performance liquid chromatography) or SE-UPLC (size exclusion ultra performance liquid chromatography), CE-SDS (capillary electrophoresis-sodium dodecyl Sulfate) or RP-HPLC (reverse phase chromatography), static light scattering (SLS), Dynamic Light Scattering (DLS), Fourier Transform Infrared Spectroscopy (FTIR), circular dichroism (CD), urea unfolding techniques, intrinsic tryptophan fluorescence, differential scanning calorimetry, and/or ANS binding techniques, compared to a reference formulation. For example, a reference formulation may be a reference standard frozen at −70° C. consisting of 10 mg/ml of an antibody (including antibody fragment thereof) in 10 mM histidine, pH 6.0-6.5 that contains 80 mM NaCl, 4% trehalose and 0.02% polysorbate 80, which reference formulation regularly gives a single monomer peak (e.g., ≥97% area) by SEC. The overall stability of a formulation comprising an antibody (including antibody fragment thereof) can be assessed by various immunological assays including, for example, ELISA and radioimmunoassay using isolated antigen molecules.

The phrase “low to undetectable levels of aggregation” as used herein refers to samples containing no more than about 5%, no more than about 4%, no more than about 3%, no more than about 2%, no more than about 1% and no more than about 0.5% aggregation by weight of protein as measured by SEC or static light scattering (SLS) techniques.

The term “low to undetectable levels of fragmentation” as used herein refers to samples containing equal to or more than about 80%, about 85%, about 90%, about 95%, about 98% or about 99% of the total protein, for example, in a single peak as determined by SEC or reverse phase chromatography, or in two peaks (e.g., heavy- and light-chains) (or as many peaks as there are subunits) by reduced Capillary Gel Electrophoresis (rCGE), representing the non-degraded antibody or a non-degraded fragment thereof, and containing no other single peaks having more than about 5%, more than about 4%, more than about 3%, more than about 2%, more than about 1%, or more than about 0.5% of the total protein in each. The term “reduced Capillary Gel Electrophoresis” as used herein refers to capillary gel electrophoresis under reducing conditions sufficient to reduce disulfide bonds in an antibody.

Concentrations, amounts, cell counts, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

### II. Antibody Formulations

The present invention provides formulations for stabilizing antibodies, in particular, as well as portions and/or fragments thereof.

### A. IVX-01101 Monoclonal Antibody Formulations

In one embodiment, the present invention provides optimized formulation for the IVX-01101, a canine monoclonal antibody comprising polypeptides that have increased binding to canine Nerve Growth Factor (NGF) and FcRn as compared to control polypeptides (e.g., the wild type counterpart IgG canine Fc regions). In certain aspects, the invention provides stabilized liquid polypeptide formulations for therapeutic use. In particular, the invention provides for the stabilization of antibodies, and antigen-binding fragments and variants thereof, for the use in treating diseases and/or disorders. In particular, the invention provides formulations that are stabilized such that the active therapeutic polypeptide is stable over an extended period of time and can be administered through a variety of administration routes. In particular, the formulations are suitable for parenteral administration. In other aspects, the invention provides a uniquely stable antibody formulation that, for example, is stable to various stresses such as freezing, lyophilization, heat, reconstitution, residual metal induced degradation and other stresses that the product may encounter during storage and administration of the antibody. Moreover, exemplary formulations of the present invention are capable of maintaining the stability, biological activity, purity and quality of the antibody or antibody fragment over an extended period of time (for example, a year or more during which time the formulation is stored) and even at unfavorable temperatures. In addition, exemplary formulations of the present invention are suitable for administration to a subject or patient, for example, parenteral administration, to a subject or patient.

### B. IVX-01286 Monoclonal Antibody Formulations

In another embodiment, the disclosure provides an evaluation of feasibility of multi-dose formulation for IVX-01286, a canine antibody that has increased binding to canine NGF for this pipeline molecules, by monitoring the product quality and stability of the molecule in different formulations with different preservatives (antimicrobials) at varying concentrations. IVX-01286 protein is a model IgG monoclonal antibody (mAb) molecule for this pipeline of canine and feline monoclonal antibodies for animal health and has increased binding to canine FcRn.

In this study, different antibody concentrations, pH/buffer systems and a variety of widely accepted parenteral preservatives (antimicrobials) were evaluated for protein quality and stability under ICH recommended stability temperatures. Based on the study results, IVX-01286 molecule formulated in 20 mM Histidine buffer, 5% (w/v) sorbitol, 0.02% (w/v) PS80, 0.05 mM EDTA, 0.9% (w/v) benzyl alcohol, pH 6.6 showed relatively good thermal stability and stability profile is similar to the molecule in the formulation without preservative, i.e. 20 mM Histidine buffer, 5% (w/v) sorbitol, 0.02% (w/v) PS80, 0.05 mM EDTA, pH 6.6.

### C. IVX-03023 Monoclonal Antibody Formulations

In yet another embodiment, the disclosure provides an evaluation of feasibility of single dose formulation for IVX-03023, a feline monoclonal antibody developed to bind with increased affinity to feline Interleukin-5 (IL-5) and FcRn as compared to control polypeptides (e.g., the wild type counterpart IgG canine or feline Fc regions). The disclosure provides proof of concept studies using the formulation of material generated using pool of clones generated by stable expression system. Product stability was monitored using multiple analytical methods and found to be stable at −70° C., 5° C., 25° C. and 40° C. well above the acceptance criteria for all the product quality attributes.

### D. IVX-06076 Monoclonal Antibody Formulations

In one other embodiment, the disclosure provides an evaluation of feasibility of single-dose formulation for IVX-06076, a feline monoclonal antibody comprising polypeptides that have increased binding to feline Nerve Growth Factor (NGF) and FcRn as compared to control polypeptides (e.g., the wild type counterpart IgG feline Fc regions). The disclosure provides proof of concept studies using the formulation of material generated using pool of clones generated by stable expression system. Product stability was monitored using multiple analytical methods and found to be stable at −70° C., 5° C., 25° C. and 40° C. well above the acceptance criteria for all the product quality attributes.

### III. Methods of Making

### A. Antibodies

A formulation according to the present invention stably supports high concentrations of bioactive antibodies in solution and offers the advantage of being suitable for parenteral administration including intravenous, intramuscular, intraperitoneal, intradermal, or subcutaneous injection. The formulation also minimizes risk of bubble formation and anaphylactoid side effects.

The antibody is preferably a monoclonal antibody, a polyclonal antibody, an antibody fragment (eg, Fab, Fab′, F (ab′) 2, Fv, Fc, ScFv, etc.), chimeric antibody, bispecific antibody, heteroconjugate antibody Single chain (ScFv), mutants thereof, fusion proteins comprising antibody portions (eg domain antibodies), humanized antibodies, human antibodies, and glycosylation variants of antibodies, amino acid sequence variants of antibodies, and covalent antibody selected from the group of any other modified configuration of an immunoglobulin molecule containing an antigen recognition site of the required specificity. The antibody can be from murine, rat, human, canine, production animal or any other source (including chimeric or humanized antibodies). In some embodiments, the antibody can be human antibody, or a humanized antibody. In other embodiments, the antibody can be a canine antibody, or a caninized antibody Preferably, the antibody is isolated and more preferably is substantially pure. Where the antibody is an antibody fragment, it preferably retains the functional characteristics of the original antibody, for example, ligand binding and/or antagonism or action activity.

### 1. Polyclonal Antibodies

Polyclonal antibodies can be prepared by immunizing a suitable subject with an immunogen. The antibody titer in the immunized subject can be monitored over time by standard techniques, such as with an enzyme linked immunosorbent assay (ELISA) using immobilized target antigen. If desired, the antibody molecules directed against the target antigen can be isolated from the mammal (for example, from the blood) and further purified by well-known techniques, such as protein A Sepharose chromatography to obtain the antibody, for example, IgG, fraction. At an appropriate time after immunization, for example, when the anti-antigen antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75). For the preparation of chimeric polyclonal antibodies, see Buechler et al. U.S. Pat. No. 6,420,113.

### 2. Monoclonal Antibodies

Any of the many well-known protocols used for fusing lymphocytes and immortalized cell lines can be applied for the purpose of generating a monoclonal antibody (see, for example, G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (for example, a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, for example, the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind a target antigen using a standard ELISA assay.

### 3. Recombinant Antibodies

As an alternative to preparing monoclonal antibody-secreting hybridomas, a monoclonal antibody can be identified and isolated by screening a recombinant combinatorial immunoglobulin library (for example, an antibody phage display library) with a target antigen to thereby isolate immunoglobulin library members that bind the target antigen. Kits for generating and screening phage display libraries are commercially available (for example, the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrad et al. (1991) Bio/Technology 9:1373-1377; Hoogenboom et al. (1991) Nuc. Acid Res 19:4133-4137; Barbas et al. (1991) Proc. Natl. Acad. Sci. USA 88:7978-7982; and McCafferty et al. Nature (1990) 348:552-554.

### 4. Chimeric and Humanized Antibodies

Additionally, recombinant antibodies, such as chimeric, humanized, felinized, caninized and the like, and the monoclonal antibodies comprising both human and non-human portions, which can be made using standard recombinant DNA techniques, are within the scope of the invention.

The term “humanized immunoglobulin” or “humanized antibody” refers to an immunoglobulin or antibody that includes at least one humanized immunoglobulin or antibody chain (i.e., at least one humanized light or heavy chain). The term “humanized immunoglobulin chain” or “humanized antibody chain” (i.e., a “humanized immunoglobulin light chain” or “humanized immunoglobulin heavy chain”) refers to an immunoglobulin or antibody chain (i.e., a light or heavy chain, respectively) having a variable region that includes a variable framework region substantially from a human immunoglobulin or antibody and complementarity determining regions (CDRs) (for example, at least one CDR, preferably two CDRs, more preferably three CDRs) substantially from a non-human immunoglobulin or antibody, and further includes constant regions (for example, at least one constant region or portion thereof, in the case of a light chain, and three constant regions in the case of a heavy chain). The term “humanized variable region” (for example, “humanized light chain variable region” or “humanized heavy chain variable region”) refers to a variable region that includes a variable framework region substantially from a human immunoglobulin or antibody and complementarity determining regions (CDRs) substantially from a non-human immunoglobulin or antibody.

The phrase “substantially from a human immunoglobulin or antibody” or “substantially human” means that, when aligned to a human immunoglobulin or antibody amino sequence for comparison purposes, the region shares at least 80-90%, 90-95%, or 95-99% identity (i.e., local sequence identity) with the human framework or constant region sequence, allowing, for example, for conservative substitutions, consensus sequence substitutions, germline substitutions, backmutations, and the like. The introduction of conservative substitutions, consensus sequence substitutions, germline substitutions, backmutations, and the like, is often referred to as ““optimization” of a humanized antibody or chain. The phrase “substantially from a non-human immunoglobulin or antibody” or “substantially non-human” means having an immunoglobulin or antibody sequence at least 80-95%, preferably at least 90-95%, more preferably, 96%, 97%, 98%, or 99% identical to that of a non-human organism, for example, a non-human mammal.

Accordingly, all regions or residues of a humanized immunoglobulin or antibody, or of a humanized immunoglobulin or antibody chain, except the CDRs, are substantially identical to the corresponding regions or residues of one or more native human immunoglobulin sequences. The term “corresponding region” or “corresponding residue” refers to a region or residue on a second amino acid or nucleotide sequence which occupies the same (i.e., equivalent) position as a region or residue on a first amino acid or nucleotide sequence, when the first and second sequences are optimally aligned for comparison purposes.

The term “significant identity” means that two polypeptide sequences, when optimally aligned, such as by the programs GAP or BESTFIT using default gap weights, share at least 50-60% sequence identity, preferably at least 60-70% sequence identity, more preferably at least 70-80% sequence identity, more preferably at least 80-90% sequence identity, even more preferably at least 90-95% sequence identity, and even more preferably at least 95% sequence identity or more (for example, 99% sequence identity or more). The term “substantial identity” means that two polypeptide sequences, when optimally aligned, such as by the programs GAP or BESTFIT using default gap weights, share at least 80-90% sequence identity, preferably at least 90-95% sequence identity, and more preferably at least 95% sequence identity or more (for example, 99% sequence identity or more). For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, for example, by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., Current Protocols in Molecular Biology). One example of algorithm that is suitable for determining percent sequence identity and sequence similarity is the BLAST algorithm, which is described in Altschul et al., J. Mol. Biol. 215:403 (1990). Software for performing BLAST analyses is publicly available through the National Center for Biotechnology Information (publicly accessible through the National Institutes of Health NCBI internet server). Typically, default program parameters can be used to perform the sequence comparison, although customized parameters can also be used. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

Preferably, residue positions which are not identical differ by conservative amino acid substitutions. For purposes of classifying amino acids substitutions as conservative or nonconservative, amino acids are grouped as follows: Group I (hydrophobic sidechains): leu, met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same class. Non-conservative substitutions constitute exchanging a member of one of these classes for a member of another.

Preferably, humanized immunoglobulins or antibodies bind antigen with an affinity that is within a factor of three, four, or five of that of the corresponding nonhumanized antibody. For example, if the nonhumanized antibody has a binding affinity of 10⁻⁹ M, humanized antibodies will have a binding affinity of at least 3×10⁻⁸M, 4×10⁻⁸ M, 5×10⁻⁸ M, or 10⁻⁹ M. When describing the binding properties of an immunoglobulin or antibody chain, the chain can be described based on its ability to “direct antigen binding”. A chain is said to “direct antigen binding” when it confers upon an intact immunoglobulin or antibody (or antigen binding fragment thereof) a specific binding property or binding affinity. A mutation (for example, a backmutation) is said to substantially affect the ability of a heavy or light chain to direct antigen binding if it affects (for example, decreases) the binding affinity of an intact immunoglobulin or antibody (or antigen binding fragment thereof) comprising said chain by at least an order of magnitude compared to that of the antibody (or antigen binding fragment thereof) comprising an equivalent chain lacking said mutation. A mutation “does not substantially affect (for example, decrease) the ability of a chain to direct antigen binding” if it affects (for example, decreases) the binding affinity of an intact immunoglobulin or antibody (or antigen binding fragment thereof) comprising said chain by only a factor of two, three, or four of that of the antibody (or antigen binding fragment thereof) comprising an equivalent chain lacking said mutation.

The term “chimeric immunoglobulin” or antibody refers to an immunoglobulin or antibody whose variable regions derive from a first species and whose constant regions derive from a second species. Chimeric immunoglobulins or antibodies can be constructed, for example by genetic engineering, from immunoglobulin gene segments belonging to different species. The terms “humanized immunoglobulin” or “humanized antibody” are not intended to encompass chimeric immunoglobulins or antibodies, as defined infra. Although humanized immunoglobulins or antibodies are chimeric in their construction (i.e., comprise regions from more than one species of protein), they include additional features (i.e., variable regions comprising donor CDR residues and acceptor framework residues) not found in chimeric immunoglobulins or antibodies, as defined herein.

Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Seidler et al. (1988) J. Immunol. 141:4053-4060.

5. Human Antibodies from Transgenic Animals and Phage Display

Alternatively, it is now possible to produce transgenic animals (for example, mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice results in the production of human antibodies upon antigen challenge. See, for example, U.S. Pat. Nos. 6,150,584; 6,114,598; and 5,770,429.

Fully human antibodies can also be derived from phage-display libraries (Hoogenboom et al., J. Mol. Biol., 227:381 (1991); Marks et al., J. Mol. Biol., 222:581-597 (1991)). Chimeric polyclonal antibodies can also be obtained from phage display libraries (Buechler et al. U.S. Pat. No. 6,420,113).

### 6. Bispecific Antibodies, Antibody Fusion Polypeptides, and Single-Chain Antibodies

Bispecific antibodies (BsAbs) are antibodies that have binding specificities for at least two different epitopes. Such antibodies can be derived from full length antibodies or antibody fragments (for example F(ab)′2 bispecific antibodies). Methods for making bispecific antibodies are known in the art. Traditional production of full length bispecific antibodies is based on the coexpression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of different antibody molecules (see, WO 93/08829 and in Traunecker et al., EMBO J., 10:3655-3659 (1991)).

Bispecific antibodies also include cross-linked or “heteroconjugate” antibodies. For example, one of the antibodies in the heteroconjugate can be coupled to avidin, the other to biotin or other payload. Heteroconjugate antibodies may be made using any convenient cross-linking methods. Suitable cross-linking agents are well known in the art, and are disclosed in U.S. Pat. No. 4,676,980, along with a number of cross-linking techniques.

In yet another embodiment, the antibody can be fused, chemically or genetically, to a payload such as a reactive, detectable, or functional moiety, for example, an immunotoxin to produce an antibody fusion polypeptide. Such payloads include, for example, immunotoxins, chemotherapeutics, and radioisotopes, all of which are well-known in the art.

Single chain antibodies are also suitable for stabilization according to the invention. The fragments comprise a heavy-chain variable domain (VH) connected to a light-chain variable domain (VL) with a linker, which allows each variable region to interface with each other and recreate the antigen binding pocket of the parent antibody from which the VL and VH regions are derived. See Gruber et al., J. Immunol., 152:5368 (1994).

### 7. Canine Antibodies

With the increasing use of polypeptide (e.g., antibodies, ligand-binding domains of receptors, enzymes, ligands, peptides) as therapeutics for the prevention and treatment of a wide variety of canine diseases, it is important to develop polypeptides with extended half-life, especially for the prevention or treatment of chronic diseases in which a polypeptide must be administered repetitively.

In canine, there are four IgG heavy chains referred to as A, B, C, and D. These heavy chains represent four different subclasses of dog IgG, which are referred to as IgGA, IgGB, IgGC and IgGD. The DNA and amino acid sequences of these four heavy chains were first identified by Tang et al. [Vet. Immunol. Immunopathol. 80: 259-270 (2001)]. The amino acid and DNA sequences for these heavy chains are also available from the GenBank data bases. For example, the amino acid sequence of IgGA heavy chain has accession number AAL35301.1, IgGB has accession number AAL35302.1, IgGC has accession number AAL35303.1, and IgGD has accession number (AAL35304.1). Canine antibodies also contain two types of light chains, kappa and lambda. The DNA and amino acid sequence of these light chains can be obtained from GenBank Databases. For example the kappa light chain amino acid sequence has accession number ABY 57289.1 and the lambda light chain has accession number ABY 55569.1.

a. Canine IgGB Antibody Fc Region

Provided herein are stabilizing formulations relating to antibodies comprising canine CH2, CH3, Fc (e.g., canine IgG Fc region variant) or canine FcRn binding fragments thereof. This disclosure features formulations for stabilizing antibodies comprising polypeptides that have increased binding to canine FcRn than control polypeptides (e.g., the wild type counterpart IgG canine Fc regions). In some instances, these polypeptides have increased binding to canine FcRn than control polypeptides at any pH (e.g., at any pH between about 5.0 to about 8.0). In some instances, these polypeptides have increased binding to canine FcRn than control polypeptides at pH 5.5, pH 6.0 and/or pH 6.5. In some instances, these polypeptides can, e.g., bind to canine FcRn at a higher level at acidic pH (e.g., pH 5.5, pH 6.0 or pH 6.5) than at a neutral pH (e.g., pH 7.0, 7.1, 7.2, 7.3, 7.4, or 7.5). In some instances, these polypeptides bind to canine FcRn at a higher level at pH 5.5, pH 6.0 or pH 6.5 than at pH 7.4. This disclosure relates, in part, to formulations for stabilizing antibodies comprising polypeptides that have increased half-life in canines than their wild type counterparts. For example, provided are binding molecules (e.g., antibodies or ligand-binding portions of receptors) with increased half-life relative to versions of these binding molecules not attached to the Fc regions (e.g., CH2, CH3, or CH2+CH3 regions) or canine FcRn binding regions disclosed herein. Also provided are stabilizing formulations for enzyme-Fc region fusions, ligand-Fc region fusions, nanobody-Fc fusions, and peptide-Fc region fusions, wherein the fusions have increased half-life compared with their wild type counterparts. The Fc regions, in addition to having a substitution or substitutions (relative to the wild type canine Fc region) that increase half-life may also include other substitutions that, e.g., increase effector function, decrease effector function, increase binding to Protein A and/or decrease heterogeneity of the polypeptide (e.g., by removing one or more post-translational modifications in the Fc region). The canine CH2, CH3, and Fc region sequences can be from any canine antibody. In some instances, the canine CH2, CH3, and Fc region sequences are from a canine IgG (e.g., IgG.A, IgG.B, IgG.C, or IgG.D). In other instances, the canine antibody is IVX-01101.

b. Feline Antibodies

Cats typically have three IgG heavy chains referred to as IgG1a, IgG1b and IgG2. These heavy chains represent three different subclasses of cat IgG. The amino acid and DNA sequences for these heavy chains are available from Strietzel et al., 2014, Vet. Immunol. Immunopathol., 158:214-223 and the GENBANK database. For example, the amino acid sequence of feline IgG1a heavy chain has GENBANK accession number BAA32229.1, feline IgG1b heavy chain has GENBANK accession number BAA32230.1, and feline IgG2 heavy chain has GENBANK accession number KF811175.1. Feline antibodies also include two types of light chains: kappa and lambda. The DNA and amino acid sequence of these light chains can also be obtained from GENBANK database. For example, the feline kappa light chain amino acid sequence has accession number AF198257.1 and the feline lambda light chain has accession number E07339.1. Feline antibodies are more specifically described in U.S. Pat. No. 11,498,953 to Brondyk et al; the contents of which is incorporated herein in its entirety.

It is understood that any of the foregoing polypeptide molecules, alone or in combination, are suitable for preparation as stabilized formulations according to the invention.

### B. Excipients

In various embodiments, the present invention provides a formulation that may include various excipients, including, but not limited to, buffer, anti-oxidant, a tonicity agent, and a stabilizer. In addition, the formulations may contain an additional agent for pH adjustment (for example, HCl) and a diluent (for example, water). In other embodiment, different forms of histidine can be used for pH adjustment. In part, the excipients serve to maintain the stability and the biological activity of the antibody (for example, by maintaining the proper conformation of the protein), and/or to maintain pH.

### 1. Buffering Agent

In various aspects of the present invention, a formulation in accordance with the present invention includes a buffering agent (buffer). The buffer serves to maintain a physiologically suitable pH, but in addition, the buffer can serve to enhance isotonicity and chemical stability of the formulation. According to the present invention the buffer provides the liquid composition with a pH close to physiological pH for reduced risk of pain or anaphylactoid side effects on injection and also provides enhanced antibody stability and resistance to aggregation, oxidation and fragmentation. In various embodiments of the present invention, the formulation has a pH of about 4.0 to about 7.2, about 5.5 to about 6.5, preferably about 6.0 to about 6.5. In a particular embodiment, the formulation has a pH of about 6. Ranges intermediate to the above recited pH levels, for example, about pH 4.0 to about pH 7.2, preferably pH 6.0, pH 6.1, pH 6.2, pH 6.3, pH 6.4, pH 6.5, pH 6.6, pH 6.7 or pH 6.8), are also intended to be part of this invention. For example, ranges of values using a combination of any of the above recited values as upper and/or lower limits are intended to be included. The pH may be adjusted as necessary by techniques known in the art. For example, HCl may be added as necessary to adjust the pH to desired levels or different forms of histidine may be used to adjust the pH to desired levels.

The buffer may include, but is not limited to, acetate (sodium or potassium), succinate (sodium or potassium), histidine, phosphate (sodium or potassium), Tris (tris (hydroxymethyl) aminomethane), diethanolamine, citrate, other organic acids, amino acids such as proline, arginine and salts, and mixtures thereof. In one embodiment, the buffer is histidine (for example, L-histidine). In another particular embodiment, the buffer is succinate. In yet another embodiment, the formulation includes an amino acid including but not limited to histidine that is present in an amount sufficient to maintain the formulation at a physiologically suitable pH. Histidine is an exemplary amino acid having buffering capabilities in the physiological pH range. Histidine derives its buffering capabilities spanning from its imidazole group. In one exemplary embodiment, the buffer is L-histidine (base) (for example C₆H₉N₃O₂, FW: 155.15). In another embodiment, the buffer is L-histidine monochloride monohydrate (for example C₆H₉N₃O₂·HCl·H₂O, FW: 209.63). In another embodiment, the buffer is L-histidine hydrochloride (for example C₆H₁₀ClN₃O₂, FW 191.61). In another exemplary embodiment, the buffer is a mixture of L-histidine (base) and L-histidine monochloride monohydrate.

In one embodiment, the buffer (for example, L-histidine or acetate or succinate) concentration is present from about 0.1 mM to about 50 mM, from about 0.1 mM to about 40 mM, from about 0.1 mM to about 30 mM, about 0.1 mM to about 25 mM, from about 0.1 mM to about 20 mM, or from about 5 mM to about 15 mM, preferably 15 mM or 25 mM. In various embodiments, the buffer may be present at about 15 mM, 16 mM, 17 mM, 18 mM, 19 mM, 20 mM, 21 mM, 22 mM, 23 mM, 24 mM, or 25 mM. In a particular embodiment, the buffer is present at about 18 mM, 19 mM, 20 mM, 21 mM, or 22 mM. Ranges intermediate to the above recited concentrations, for example, about 16 mM to about 24 mM, are also intended to be part of this invention. For example, ranges of values using a combination of any of the above recited values as upper and/or lower limits are intended to be included. In certain embodiments, the buffer is present in an amount sufficient to maintain a physiologically suitable pH.

2. Tonicity and/or Stabilizing Agent

In various aspects of the present invention, the formulation includes a stabilizer that can also act as tonicity agent. In part, the tonicity agent contributes to maintaining the isotonicity of the formulation, and to maintaining protein levels. In part, the stabilizing agent contributes to preserving the level, ratio, or proportion of the therapeutically active polypeptide present in the formulation. As used herein, the term “tonicity” refers to the behavior of biologic components in a fluid environment or solution. Isotonic solutions possess the same osmotic pressure as blood plasma, and so can be intravenously infused into a subject without changing the osmotic pressure of the subject's blood plasma. Indeed, in one embodiment according to the invention, tonicity and/or stabilizing agent is present in an amount sufficient to render the formulation suitable for intravenous infusion. Often, the tonicity agent serves as a stabilizing agent as well. As such, the agent may allow the protein to overcome various stresses such as freezing, thawing, transport, high temperatures and shear.

The tonicity and/or stabilizing agent may include, but is not limited to, CaCl₂), NaCl, MgCl₂, lactose, sorbitol, sucrose, mannitol, trehalose, raffinose, polyethylene glycol, hydroxyethyl starch, glycine and mixtures thereof. In a preferred embodiment, the tonicity and/or stabilizing agent is sucrose, trehalose, or sorbitol. According to the present invention the stabilizing agent provides the liquid composition with enhanced antibody stability and resistance to aggregation, oxidation and fragmentation during refrigerated storage, e.g. 0 to 10° C., particularly 5 to 8° C., more particularly 5° C., or frozen storage and in cycles of freezing and thawing.

In one embodiment, the tonicity and/or stabilizing agent is present at about 0.1% to about 35% w/v, or about 3% to about 25% w/v. In another embodiment, the tonicity agent is present at about 5% to about 15% w/v. In another embodiment, the tonicity agent is present at about 8% to about 10% w/v. In another embodiment, the tonicity agent is percent at about 20 mg/ml to about 60 mg/ml, at about 30 mg/ml to about 50 mg/ml, or at about 35 mg/ml to about 45 mg/ml. Preferably, the tonicity agent is present at about 4% w/v. In another embodiment, the tonicity agent is present at about 5% w/v. In another particular embodiment, the tonicity agent is present at about 6% w/v. In another particular embodiment, the tonicity agent is present at about 7% w/v. In yet particular embodiment, the tonicity agent is present at about 8% w/v. In another particular embodiment, the tonicity agent is present at about 9% w/v. In still another particular embodiment, the tonicity agent is present at about 10% w/v.

Ranges intermediate to the above recited concentrations, for example, about 3% to about 12% w/v, are also intended to be part of this invention. For example, ranges of values using a combination of any of the above recited values as upper and/or lower limits are intended to be included. The tonicity and/or stabilizing agent should be present in a sufficient amount so as to maintain tonicity of the formulation.

### 3. Chelating Agent

In various aspects of the present invention, the formulation includes a chelating agent that provides the liquid composition with an enhanced antibody stability and/or resistance to aggregation. Chelating agents can lower the formation of reduced oxygen species, reduce acidic species (e.g., deamidation) formation, reduce antibody aggregation, and/or reduce antibody fragmentation, and/or reduce antibody oxidation in the compositions of the present invention. Such chelating agents can reduce or prevent degradation of an antibody that is formulated in comparison to the antibody without the protection of a chelating agent.

According to one embodiment of the present invention, the chelating agent can be selected from the group consisting of aminopolycarboxylic acids, hydroxyaminocarboxylic acids, N-substituted glycines, 2-(2-amino-2-oxoethyl) aminoethane sulfonic acid (BES), deferoxamine (DEF), citric acid, niacinamide, and desoxycholates and mixtures thereof. A further embodiment contemplates that the chelating agent is selected from the group consisting of ethylenediaminetetraacetic acid (EDTA), diethylenetriamine pentaacetic acid 5 (DTPA), nitrilotriacetic acid (NTA), N-2-acetamido-2-iminodiacetic acid (ADA), bis(aminoethyl)glycolether, N,N,N′,N′-tetraacetic acid (EGTA), trans-diaminocyclohexane tetraacetic acid (DCTA), glutamic acid, and aspartic acid, N-hydroxyethyliminodiacetic acid (HIMDA), N,N-bis-hydroxyethylglycine (bicine) and N-(trishydroxymethylmethyl) 10 glycine (tricine), glycylglycine, sodium desoxycholate, ethylenediamine; propylenediamine; diethylenetriamine; triethylenetetraamine (trien), ethylenediaminetetraaceto EDTA; disodium EDTA, calcium EDTA, oxalic acid, malate, citric acid, citric acid monohydrate, and trisodium citrate-dihydrate, 8-hydroxyquinolate, amino acids, histidine, cysteine, methionine, peptides, polypeptides, and proteins and mixtures thereof. In yet another aspect of the present invention, the chelating agent is selected from the group consisting of salts of EDTA including dipotassium edetate, disodium edetate, edetate calcium disodium, sodium edetate, trisodium edetate, and potassium edetate; and a suitable salt of deferoxamine (DEF) is deferoxamine mesylate (DFM), or mixtures thereof. Chelating agents used in the invention can be present, where possible, as the free acid or free base form or salt form of the compound, also as an anhydrous, solvated or hydrated form of the compound or corresponding salt.

In still a further embodiment of the present invention, the chelating agent is disodium EDTA or calcium EDTA.

The concentration of chelating agent generally ranges from about 0.01 mM to about 50 mM from about 1 mM to about 10.0 mM, from about 15 mM to about 5.0 mM, from about 0.01 mM to about 1.0 mM, or from about 0.03 mM to about 0.5 mM. In one embodiment, the concentration of chelating agent generally ranges from about 0.01 mM to about 2.0 mM, from about 0.01 mM to about 1.5 mM, from about 0.01 mM to about 0.5 mM, from about 0.01 mM to about 0.4 mM, from about 0.01 mM to about 0.3 mM, from about 0.01 mM to about 0.2 mM, from about 0.01 mM to about 0.15 mM, from about 0.01 mM to about 0.1 mM, from about 0.01 mM to about 0.09 mM, from about 0.01 mM to about 0.08 mM, from about 0.01 mM to about 007 mM, from about 0.01 mM to about 0.06 mM, from about 0.01 mM to about 0.05 mM, from about 0.01 mM to about 0.04 mM, from about 0.01 mM to about 0.03 mM, from about 0.01 mM to about 0.02 mM or from about 0.05 mM to about 0.01 mM. In another embodiment the concentration of chelating agent can be about 0.01 mg/ml, 0.02 mg/ml, 0.03 mg/ml, about 0.04 mg/ml, about 0.05 mg/ml, about 0.06 mg/ml, about 0.07 mg/ml, about 0.10 mg/ml, about 0.20 mg/ml. Further embodiments for the concentration of chelating agent contemplate using about 0.045 mM, about 0.046 mM, about 0.047 mM, about 0.048 mM, about 0.049 mM, about 0.05 mM, about 0.051 mM, about 0.052 mM, about 0.053 mM, about 0.054 mM, about 0.055 mM, or about 0.056 mM. Ranges intermediate to the above recited concentrations, for example, about 0.04 mM to about 0.06 mM, are also intended to be part of this invention.

Unless stated otherwise, the concentrations listed herein are those concentrations at ambient conditions, [i.e., at 25° C. and atmospheric pressure].

### 4. Antioxidant Agent

In various aspects of the present invention, the formulation includes an antioxidant agent, so as to, in part, preserve the formulation (for example, by preventing oxidation). Preferably the antioxidant is selected from the group comprising, methionine, sodium thiosulfate, catalase, and platinum.

The anti-oxidant may include, but is not limited to, GLA (gamma-linolenic acid)-lipoic acid, DHA (docosahexaenoic acid)-lipoic acid, GLA-tocopherol, di-GLA-3,3′-thiodipropionic acid and in general any of, for example, GLA, DGLA (dihomo-gamma-linolenic acid), AA (arachidonic acid), SA (salicylic acid), EPA (eicosapentaenoic acid) or DHA (docosahexaenoic acid) with any natural or synthetic anti-oxidant with which they can be chemically linked. These include phenolic anti-oxidants (for example, eugenol, carnosic acid, caffeic acid, BHT (butylated hydroxyanisol), gallic acid, tocopherols, tocotrienols and flavenoid anti-oxidants (such as myricetin and fisetin)), polyenes (for example, retinoic acid), unsaturated sterols (for example, Δ⁵-avenosterol), organosulfur compounds (for example, allicin), terpenes (for example, geraniol, abietic acid) and amino acid antioxidants (for example, methionine, cysteine, carnosine). In one embodiment, the anti-oxidant is ascorbic acid. In another embodiment, the anti-oxidant is methionine, or an analog thereof, for example, selenomethionine, hydroxy methyl butanoic acid, ethionine, or trifluoromethionine.

In one embodiment, the anti-oxidant is present from about 0.1 mM to about 50 mM, from about 0.1-mM to about 40 mM, from about 0.1 mM to about 30 mM, from about 0.1 mM to about 20 mM, or from about 5 mM to about 15 mM. In various embodiments, the anti-oxidant may be present at about 2 mM, 3 mM, 4 mM, 5 mM, 6 mM, 7 mM, 8 mM, 9 mM, 10 mM, 11 mM, 12 mM, 13 mM, 14 mM, or 15 mM. In a particular embodiment, the anti-oxidant is present at about 5 mM. In another particular embodiment, the anti-oxidant is present at about 10 mM. In yet another particular embodiment, the anti-oxidant is present at about 15 mM. Ranges intermediate to the above recited concentrations, for example, about 3 mM to about 17 mM, are also intended to be part of this invention. For example, ranges of values using a combination of any of the above recited values as upper and/or lower limits are intended to be included. In certain embodiments, the anti-oxidant should be present in a sufficient amount so as to preserve the formulation, in part, by preventing oxidation.

### 5. Surfactant Agent

In various aspects of the present invention, the formulation includes a surfactant agent which, in accordance with the present invention provides the liquid composition with enhanced antibody stability and resistance to aggregation and fragmentation.

According to a preferred embodiment of the present invention the surfactant is preferably selected from the group consisting of polysorbates, poloxamers, tritons, sodium dodecyl sulfate, sodium laurel sulfate, sodium octyl glycoside, lauryl-sulfobetaine, myristyl-sulfobetaine, linoleyl-sulfobetaine, stearyl-sulfobetaine, lauryl-sarcosine, myristyl-sarcosine, linoleyl-sarcosine, stearyl-sarcosine, linoleyl-betaine, myristyl-betaine, cetyl-betaine, lauroamidopropyl-betaine, cocamidopropyl-betaine, linoleamidopropyl-betaine, myristamidopropyl-betaine, palmidopropyl-betaine, isostearamidopropyl-betaine, myristamidopropyl-dimethylamine, palmidopropyl-dimethylamine, isostearamidopropyl-dimethylamine, sodium methyl cocoyl-taurate, disodium methyl oleyl-taurate, dihydroxypropyl PEG 5 linoleammonium chloride, polyethylene glycol, polypropylene glycol, and mixtures thereof. Further preferably the surfactant is selected from the group consisting of polysorbate 20, polysorbate 21, polysorbate 40, polysorbate 60, polysorbate 61, polysorbate 65, polysorbate 80, polysorbate 81, polysorbate 85, and mixtures thereof. More preferably the surfactant is selected from polysorbate 20, polysorbate 80, PEG3350, or mixtures thereof.

The concentration of the surfactant generally ranges from about 0.01% to about 10% (w/v), from about 0.01% to about 5.0%, from about 0.01% to about 2.0 mg/ml, from about 0.01% to about 1.5%, from about 0.01% to about 1.0%, from about 0.01% to about 0.5%, from about 0.01% to about 0.4%, from about 0.01% to about 0.3%, from about 0.01% to about 0.2%, from about 0.01% to about 0.15%, from about 0.01% to about 0.1%, or from about 0.01%, to about 0.05%. Further preferably the concentration of the surfactant is about 0.01%, about 0.02%, about 0.03%, about 0.04%, about 0.05%, about 0.06%, about 0.07%, about 0.08%, about 0.09%, about 0.10%, about 0.11%, about, 0.12% about 0.13%, about 0.14%, about 0.15%, about 0.16%, about 0.17%. Most preferably the concentration of the surfactant is about 0.02%. Embodiments with the concentration of the surfactant of 0.01%, about 0.02%, about 0.03%, about 0.04%, or about 0.05% are highly preferred as this concentration permits maintenance of the stability of the antibody of the formulation in solution whilst also reducing the tendency for the formation of bubbles in the formulation during preparation of the formulation, handling of the formulation and preparation for parenteral administration and especially from stress related to shaking and agitation during preparation and also during shipping.

### 5. Preservative

In various aspects of the present invention the formulation includes an anti-microbial preservative. In one embodiment, the preservative agent is selected from phenol, m-cresol, benzyl alcohol, benzalkonium chloride, benzalthonium chloride, phenoxyethanol and methyl paraben or mixtures thereof.

The concentration of preservative generally ranges from about 0.001% w/v to 10% w/v, from about 0.005% w/v to about 5% w/v, from about 0.008 w/v to about 2.0% w/v or from about 0.01% w/v to about 1.5% w/v. Preferably the concentration of preservative can be about 0.1% w/v, 0.2% w/v, 0.3% w/v, about 0.4% w/v, about 0.5% w/v, about 0.6% w/v, about 0.7% w/v, 0.8% w/v, 0.9% w/v about 1.0% w/v, 2.0% w/v, 3.0% w/v, about 4.0% w/v, about 5.0% w/v, about 6.0% w/v, about 7.0% w/v, 8.0% w/v, 9.0% w/v about 9.1% w/v, about 9.2% w/v, 9.3% w/v, 9.4% w/v, 9.5% w/v, 9.6% w/v, 9.7% w/v, 9.8% w/v, 9.9% w/v, 10.0% w/v. In particular embodiments, the concentration of preservative is about 0.01% w/v or 9.0% w/v.

### C. Pharmaceutical Compositions

To prepare pharmaceutical or sterile compositions of an antibody or antigen binding fragment thereof, the formulation can be admixed with a pharmaceutically acceptable carrier or excipient. Various references and handbooks well known in the art can be consulted for more information. See, for example, Remington's Pharmaceutical Sciences and U.S. Pharmacopeia: National Formulary, Mack Publishing Company, Easton, Pa. (1984) (the contents of which is hereby incorporated in its entirety by reference).

Formulations of therapeutic and diagnostic agents may also be prepared by mixing with acceptable carriers, excipients, or stabilizers in the form of, for example, lyophilized powders, slurries, aqueous solutions or suspensions (see, for example, Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Marcel Dekker, NY; Lieberman, et al. (eds.) (1990) Pharmaceutical Dosage Forms: Disperse Systems, Marcel Dekker, NY; and Weiner and Kotkoskie (2000) Excipient Toxicity and Safety, Marcel Dekker, Inc., New York, N.Y.]. In one embodiment, the antibodies of the present invention are diluted to an appropriate concentration in a sodium acetate solution pH 4.5-7.0, and sorbitol or sucrose is added for tonicity and/or stabilizing the protein. Additional agents, such as polysorbate 20 or polysorbate 80, may be added to enhance stability.

Toxicity and therapeutic activity or efficacy of the antibody compositions, administered alone or in combination with another agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index (LD₅₀/ED₅₀). In particular aspects, antibodies exhibiting high therapeutic indices are desirable. The data obtained from these cell culture assays and animal studies can be used in formulating a range of dosage for use in the subject, including humans, canines, felines and other mammals. The dosage of such compounds lies preferably within a range of circulating concentrations that include the ED₅₀ with little or no toxicity. The dosage may vary within this range depending upon the dosage form employed and the route of administration.

### D. Administration

The mode of administration for a formulation according to the present invention can vary. Suitable routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial. In particular embodiments, the antibody formulation of this invention, or antigen binding fragment thereof, can be administered by an parenteral route such as by injection. In further embodiments of the invention, an antibody of this invention or antigen binding fragment thereof, or pharmaceutical composition thereof, is administered intravenously, subcutaneously, intramuscularly, intraarterially, intratumorally, or by inhalation including by aerosol delivery. Administration by non-invasive routes (for example, orally including but not limited to a pill, capsule or tablet) is also within the scope of the present invention.

According to one embodiment of the present embodiment, the formulations can be administered directly into the blood stream, into muscle, into tissue, into fat, or into an internal organ. Suitable means for parenteral administration include intravenous, intraarterial, intraperitoneal, intrathecal, intraventricular, intraurethral, intrasternal, intracranial, intramuscular, intra-ossial, intradermal and subcutaneous. Suitable devices for parenteral administration include needle (including microneedle, microprojections, soluble needles and other micropore formation techniques) injectors, needle-free injectors and infusion techniques.

The pharmaceutical compositions disclosed herein may also be administered by infusion. Examples of well-known implants and modules form administering pharmaceutical compositions include: U.S. Pat. No. 4,487,603, which discloses an implantable micro-infusion pump for dispensing medication at a controlled rate; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments. Many other such implants, delivery systems, and modules are well known to those skilled in the art.

Alternately, one may administer an antibody in a local rather than systemic manner, for example, via injection of the antibody directly into an arthritic joint or pathogen-induced lesion characterized by immunopathology, often in a depot or sustained release formulation. Furthermore, one may administer the antibody in a targeted drug delivery system, for example, in a liposome coated with a tissue-specific antibody, targeting, for example, arthritic joint or pathogen-induced lesion characterized by immunopathology. The liposomes will be targeted to and taken up selectively by the afflicted tissue.

The administration regimen depends on several factors, including the serum or tissue turnover rate of the therapeutic antibody, the level of symptoms, the immunogenicity of the therapeutic antibody, and the accessibility of the target cells in the biological matrix. Preferably, the administration regimen delivers sufficient therapeutic antibody to effect improvement in the target disease state, while simultaneously minimizing undesired side effects. Accordingly, the amount of biologic delivered depends in part on the particular therapeutic antibody and the severity of the condition being treated. Guidance in selecting appropriate doses of therapeutic antibodies is available [see, e.g., Wawrzynczak Antibody Therapy, Bios Scientific Pub. Ltd, Oxfordshire, U K (1996); Kresina (ed.) Monoclonal Antibodies, Cytokines and Arthritis, Marcel Dekker, New York, N.Y. (1991); Bach (ed.) Monoclonal Antibodies and Peptide Therapy in Autoimmune Diseases, Marcel Dekker, New York, N.Y. (1993); Baert, et al. New Engl. J. Med. 348:601-608 (2003); Milgrom et al. New Engl. J. Med. 341:1966-1973 (1999); Slamon et al. New Engl. J. Med. 344:783-792 (2001); Beniaminovitz et al. New Engl. J. Med. 342:613-619 (2000); Ghosh et al. New Engl. J. Med. 348:24-32 (2003); Lipsky et al. New Engl. J. Med. 343:1594-1602 (2000)].

Determination of the appropriate dose is well understood by those of skill in the art and can be determined using well-known parameters or factors known or suspected to affect treatment. Generally, the dose begins with an amount somewhat less than the optimum dose and it is increased by small increments thereafter until the desired or optimum effect is achieved relative to any negative side effects. Important diagnostic measures include monitoring those associated with symptoms. For example, inflammation or level of inflammatory cytokines produced following treatment can be determined and/or monitored.

Antibodies, including antigen binding fragments thereof, disclosed herein may be provided by continuous infusion, or by doses administered at various intervals including daily, 1-7 times per week, weekly, bi-weekly, monthly, bimonthly, quarterly, semiannually, annually and the like. Doses may be provided via a number of routes including but not limited to intravenously, subcutaneously, topically, orally, nasally, rectally, intramuscular, intracerebrally, intraspinally, or by inhalation. A total weekly dose is generally at least 0.05 μg/kg body weight, more generally at least 0.2 μg/kg, 0.5 μg/kg, 1 μg/kg, 10 μg/kg, 100 μg/kg, 0.25 mg/kg, 1.0 mg/kg, 2.0 mg/kg, 5.0 mg/ml, 10 mg/kg, 25 mg/kg, 50 mg/kg or more [see, e.g., Yang, et al. New Engl. J. Med. 349:427-434 (2003); Herold, et al. New Engl. J. Med. 346:1692-1698 (2002); Liu, et al. J. Neurol. Neurosurg. Psych. 67:451-456 (1999); Portielji, et al. Cancer Immunol. Immunother. 52:133-144 (2003)). Doses may also be provided to achieve a pre-determined target concentration of an antibody in the subject's serum, such as 0.1, 0.3, 1, 3, 10, 30, 100,300 μg/ml or more. In other embodiments, an antibody of the present invention is administered subcutaneously or intravenously, on a weekly, biweekly, “every 4 weeks,” monthly, bimonthly, or quarterly basis at 10, 20, 50, 80, 100, 200, 500, 1000 or 2500 mg/subject.

In some embodiments the administration pattern of the medicament comprises administration of a dose of the medicament once every week, once every two weeks, once every three weeks, once every four weeks, once every five weeks, once every six weeks, once every seven weeks, once every eight weeks, once every nine weeks, once every ten weeks, once every fifteen weeks, once every twenty weeks, once every twenty five weeks, or once every twenty six weeks. In some embodiments, the anti-NGF antagonist antibody is administered once every month, once every two months, once every three months, once every four months, once every five months, or once every six months. Most preferably the administration pattern of the medicament comprises administration of a dose of the medicament once every eight weeks.

In some embodiments the volume of a dose is less than or equal to about 20 ml, about 15 ml, about 10 ml, about 5 ml, about 2.5 ml, about 1.5 ml, about 1.0 ml, about 0.75 ml, about 0.5 ml, about 0.25 ml or about 0.01 ml.

In some embodiments the volume of a dose is about 20 ml, about 19 ml, about 18 ml, about 17 ml, about 16 ml, about 15 ml, about 14 ml, about 13 ml, about 12 ml, about 11 ml, about 10 ml, about 9 ml, about 8 ml, about 7 ml, about 6 ml, about 5 ml, about 4 ml, about 3 ml, about 2 ml or about 1 ml. Alternatively about 20.5 ml, about 19.5 ml, about 18.5 ml, about 17.5 ml, about 16.5 ml, about 15.5 ml, about 14.5 ml, about 13.5 ml, about 12.5 ml, about 11.5 ml, about 10.5 ml, about 9.5 ml, about 8.5 ml, about 7.5 ml, about 6.5 ml, about 5.5 ml, about 4.5 ml, about 3.5 ml, about 2.5 ml, about 1.5 ml, or about 0.5. Alternatively about 900 microliters, about 800 microliters, about 700 microliters, about 600 microliters, about 500 microliters, about 400 microliters, about 300 microliters, about 200 microliters, or about 100 microliters, alternatively about 950 microliters, about 850 microliters, about 750 microliters, about 650 microliters, about 550 microliters, about 450 microliters, about 350 microliters, about 250 microliters, about 150 microliters, or about 50 microliters. Most preferably the volume of the dose is less than or equal to about 2.5 ml.

According to an embodiment of the present invention, the concentration of antibody can range from about 0.1 to about 200 mg/ml. Preferably the concentration of antibody is about 0.5 mg/ml, about 1 mg/ml, about 2 mg/ml, about 2.5 mg/ml, about 3 mg/ml, about 3.5 mg/ml, about 4 mg/ml, about 4.5 mg/ml, about 5 mg/ml, about 5.5 mg/ml, about 6 mg/ml, about 6.5 mg/ml, about 7 mg/ml, about 7.5 mg/ml, about 8 mg/ml, about 8.5 mg/ml, about 9 mg/ml, about 9.5 mg/ml, about 10 mg/ml, about 11 mg/ml, about 12 mg/ml, about 13 mg/ml, about 14 mg/ml, about 15 mg/ml, about 16 mg/ml, about 17 mg/ml, about 18 mg/ml, about 19 mg/ml, about 20 mg/ml, about 21 mg/ml, about 22 mg/ml, about 23 mg/ml, about 24 mg/ml, about 25 mg/ml, about 26 mg/ml, about 27 mg/ml, about 28 mg/ml, about 29 mg/ml, about 30 mg/ml, about 31 mg/ml, about 32 mg/ml, about 33 mg/ml, about 34 mg/ml, about 35 mg/ml, about 36 mg/ml, about 37 mg/ml, about 38 mg/ml, about 39 mg/ml, about 40 mg/ml, about 41 mg/ml, about 42 mg/ml, about 43 mg/ml, about 44 mg/ml, about 45 mg/ml, about 46 mg/ml, about 47 mg/ml, about 48 mg/ml, about 49 mg/ml, about 50 mg/ml, about 51 mg/ml, about 52 mg/ml, about 53 mg/ml, about 54 mg/ml, about 55 mg/ml, about 56 mg/ml, about 57 mg/ml, about 58 mg/ml, about 59 mg/ml, about 60 mg/ml, about 70 mg/ml, about 80 mg/ml, about 90 mg/ml, about 100 mg/ml or about 110 mg/ml. In one embodiment, the concentration of antibody may be selected from the group comprising about 2 mg/ml, about 2.5 mg/ml, about 5 mg/ml, about 10 mg/ml, about 20 mg/ml, about 30 mg/ml, about 40 mg/ml, about 50 mg/ml, 60 mg/ml, about 70 mg/ml, about 80 mg/ml, about 90 mg/ml, or about 100 mg/ml.

According to embodiments contemplated by the present invention, the dose contains less than or equal to about 0.5 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 70 mg, about 80 mg, about 90 mg, about 100 mg, or about 110 mg of antibody.

According to another embodiment the dose contains an amount of antibody that is about 1 μg/kg, about 10 μg/kg, about 20 μg/kg, about 25 μg/kg, about 50 μg/kg, about 100 μg/kg, about 200 μg/kg, about 250 μg/kg, about 500 μg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, or about 11 mg/kg (of mass of the mammal to which the dose it to be administered). In yet another embodiment, the dose contains about 20 μg/kg, about 25 μg/kg, about 50 μg/kg, about 100 μg/kg, about 200 μg/kg, about 250 μg/kg, 1 mg/kg or about 2 mg/kg.

Dosage regimens may vary over time depending on the pattern of pharmacokinetic decay that the practitioner wishes to achieve. For example, in some embodiments, although dosing from one-four times a week is contemplated, even less frequent dosing may be used. In some embodiments, the dose is administered once every 2 weeks, every 3 weeks, every 4 weeks, every 5 weeks, every 6 weeks, every 7 weeks, every 8 weeks, every 9 weeks, every 10 weeks, every 15 weeks, every 20 weeks, every 25 weeks, or longer. In some embodiments, the dose is administered once every 1 month, every 2 months, every 3 months, every 4 months, every 5 months, every 6 months, or longer. In yet another embodiment, the dose is administered once every eight weeks. The progress of this therapy is easily monitored by conventional techniques and assays.

For the purpose of the present invention, the appropriate dosage of the medicament will depend on the antibody employed. For example formulations comprising a polypeptide comprising a canine IgG Fc region variant, or a canine FcRn-binding region thereof, may be administered to manage pain. The dosage would depend on factors including, but not limited to, the type and severity of the pain to be treated; whether the agent is administered for preventative or therapeutic purposes; previous therapy; the patient's clinical history and response to the agent; the discretion of the attending professional; and the like. Typically the clinician will administer the medicament until a dosage is reached that achieves the desired result. Dosages may be determined empirically. For example, the animal or individual may be given incremental dosages to assess efficacy of the medicament with subsequent follow-up of an indicator(s) of pain followed as by, for example, evaluating a change in a pain numerical rating scale (NRS).

Dose and/or frequency of a formulation according to the present invention can vary over course of treatment. Empirical considerations, such as the antibody half-life, generally will contribute to the determination of the dosage. Frequency of administration may be determined and adjusted over the course of therapy, and is generally, but not necessarily, based on treatment and/or suppression and/or amelioration and/or delay of pain. In some animals or individuals more than one dose may be required. Frequency of administration may be determined and adjusted over the course of therapy. For repeated administrations over several days or longer, depending on the pain and its severity, the treatment is sustained until a desired suppression of symptoms occurs or until sufficient therapeutic levels are achieved to reduce pain.

Presentation of the formulation may be provided as a single dose or multi-dose presentation in vials or syringes. Administration of the formulation comprising the liquid composition can be continuous or intermittent, depending, for example, upon a number of factors including but not limited to the recipient's physiological condition, whether the purpose of the administration is therapeutic or prophylactic, and other factors known to skilled practitioners. The administration of the medicament comprising the liquid composition may be essentially continuous over a preselected period of time or may be in a series of spaced doses, for example, either before, during, or after developing pain.

In one embodiment, the administration of the dose is contemplated as parenteral administration preferably selected from intravenous, intraarterial, intraperitoneal, intrathecal, intraventricular, intraurethral, intrasternal, intracranial, intramuscular, intra-ossial, intradermal and subcutaneous routes. One embodiment contemplates providing the formulation in a unit dosage sterile liquid form for parenteral administration.

Treatment efficacy can be assessed by monitoring the disease or condition. For example, pain relief may be characterized by time course of relief. Accordingly, in some embodiments, relief is observed within about 24 hours after administration. In other embodiments, relief is observed within about 36, 48, 60, 72 hours or 4 days after administration. In some embodiments, frequency and/or intensity of pain is diminished, and/or quality of life of those suffering pain is increased. In some embodiments, pain relief is provided for duration of at least about 7 days, at least about 14 days, at least about 21 days, at least about 28 days, at least about 35 days, at least about 42 days, at least about 49 days, at least about 56 days, at least about 63 days, at least about 70 days, at least about 77 days, at least about 84 days, at least about 180 days, or longer after a single dose of the medicament.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

For any method disclosed herein that includes discrete steps, the steps may be conducted in any feasible order. And, as appropriate, any combination of two or more steps may be conducted simultaneously.

The description exemplifies illustrative embodiments. In several places throughout the application, guidance is provided through lists of examples, which examples can be used in various combinations. In each instance, the recited list serves only as a representative group and should not be interpreted as an exclusive list.

All headings are for the convenience of the reader and should not be used to limit the meaning of the text that follows the heading, unless so specified.

The present invention is illustrated by the following examples. It is to be understood that the particular examples, materials, amounts, and procedures are to be interpreted broadly in accordance with the scope and spirit of the invention as set forth herein.

### Examples

### Example 1: IVX-01101 Monoclonal Antibody pH/Buffer Screening Study

Provided herein is a 2-part design of the development of an antibody stabilizing formulation. The antibody used for this study is the IVX-01101 monoclonal antibody.

To start, a pH/buffer screening study was conducted under stress condition (40° C. incubation) to select the optimal pH/buffer system for stabilizing an antibody the most. Next, a screening study of excipients and surfactant strength was conducted to evaluate and select the excipients and surfactant strength according to their stabilizing effect on IVX-01101 under stress conditions (40° C. incubation, multiple freeze and thaw cycles, and agitation).

Biophysical and analytical methods included appearance, pH, protein concentration (conc.), osmolality, sub-visible particles (microflow imaging (MFI)), size exclusion-ultra performance liquid chromatography (SE-UPLC), imaged capillary isoelectric focusing (iCIEF), CE-sodium dodecyl sulfate-reduced & non-reduced (CE-SDS-R & NR), differential scanning calorimeter (DSC). Potency testing was used for the selected formulation.

The data summary (DS) information used in the formulation development study is listed in Table 1.

TABLE 1
DS information

| Material | Manufactured | | DS | |
|---|---|---|---|---|
| name | by | Lot No. | source | Formulation |
| IVX- | WuXi | SH23362110 | 3L | 20 mM histidine/histidine |
| 01101 | Biologics | | BRs | hydrochloride buffer, 8% |
| DS | | | | (w/v) sucrose, pH 6.5 |

### Materials and Methods

Appearance: The appearance of all samples, including clarity, color and visible particles, was examined against black and white backgrounds using the YB-2 Clarity Detector.

Protein Concentration: Protein concentration (conc.) was determined by a Thermo UV spectrophotometer Nano Drop One. The extinction coefficient used in all evaluation studies was 1.52 AU*mL*mg⁻¹*cm⁻¹. Measurement were repeated twice with 2.5 μL sample for each sample and an average result was reported. Each sample measurement was conducted after blank control using water.

pH: pH value was measured using a pH meter with a glass electrode. The pH meter was calibrated with three different standard (pH 4.01, 7.00 and 9.21) prior to usage. The slope of calibration was between 95.0%-105.0%. Each sample were measured twice; an average result was reported.

Osmolality: Osmolality was measured using Advanced 2020 Multi-Sample Osmometer. Before and after the tests, the testing accuracy of the osmometer was confirmed with a Clinitrol 290 mOsm/kg reference solution. The sample volume for testing was 20 μL and only one test was performed for each sample.

MFI: Sub-visible particles were monitored by a Micro Flow Imaging (MFI) system. 1.5 mL of each sample was transferred into the MFI 96-well plate in bio-safety hood for analysis. The results were analyzed by the vendor's MVAS software. The sub-visible particle amounts in the equivalent circular diameter over 2 μm, 5 μm, 10 μm, and μm were reported.

Differential Scanning Calorimeter (DSC): DSC is a thermoanalytical technique in which the difference in the amount of heat required to increase the temperature of the sample or the reference is measured as a function of temperature. Samples were diluted to 1 mg/mL with the reference buffer. 400 μL of respective reference buffers were added into the odd-numbered wells of a 96-well plate and 400 μL of samples were added into the even-numbered wells of the same plate. Experimental parameters were set such that the scan temperature ramped from 10 to 95° C. at a scan rate of 200° C./h. Data analysis was performed in Malvern VP DSC automated data analysis software.

SE-UPLC: Size exclusion ultra performance liquid hcromatograpy (SE-UPLC) is a purity analysis method that separates proteins based on their sizes. Size exclusion chromatography was performed on an Agilent UPLC system with a SEC column (Waters Acquity BEH 150×4.6 mm, 1.7 μm). The sampler temperature was set to 5±3° C. and the column oven temperature was set as 25±3° C. The mobile phase was 50 mM PB, 300 mM NaCl, pH 6.8±0.1 and the flow rate was set as 0.4 mL/min. 10 μg of each sample was injected. Detection wavelength was set at 280 nm and the run time was 8 minutes. Data was analyzed by Agilent CDS Software.

iCIEF: Imaged capillary isoelectric focusing (iCIEF) is a high-resolution technique to separate protein into groups based on their isoelectric point (pI). Compared to conventional CIEF systems, iCIEF provides faster method development, higher detection sensitivity, better reliability, and higher analytical throughput. 20 μL of the reference standard or samples (diluted to 1.0 mg/mL) were mixed individually with 80 μL of master mixture, which is composed by 0.5 μL of high pI marker (7.05), 0.5 μL low pI marker (4.22), 1.0 μL of Pharmalyte 3-10, 3.0 μL of Pharmalyte 5-8, 35 μL of 1% methyl cellulose (MC), 37.5 μL of 8 M urea solution, 2.0 μL 200 mM Iminodiacetic Acid solution and 0.5 μL of ultrapure water. The final protein concentration of sample in loading mixture is 0.2 mg/mL. The loading mixture was then analyzed with iCE3 Capillary Isoelectric Focusing Analyzer equipped with a FC-COATED whole-column detection capillary. The focusing was carried out by two steps: (1) 1.5 kV for 1 minute; (2) 3 kV for 8 minutes, and the auto-sampler tray was maintained at 10° C. Absorbance detection took place at 280 nm. After the analysis, the raw data were processed with Empower 3.

CE-SDS-NR: Non-reduced Capillary Electrophoresis Sodium Dodecyl Sulfate (CE-SDS-NR) was performed based on a PerkinElmer Caliper automated electrophoresis that allows protein separation by size and helps determine purity. In the non-reduced microchip CE-SDS analysis, samples were diluted to 1 mg/mL with pure water. 2 μL of diluted samples were mixed with 7 μL of the sample denaturing solution (the mixture of sample buffer, 10% SDS solution and 100 mM N-Ethylmaleimide (NEM) with the volume ratio of 100:10:4). The well-mixed samples were incubated at 70° C. for 10 minutes. 35 μL of water was added to each sample, vortexed for 10 seconds and centrifuged at 14000 rpm for 1 minute. 42 μL of the prepared samples were transferred to a 96-well plate, centrifuged at 4000 rpm for 15 minutes, placed onto a Labchip GXII's plate holder, separated and detected in the HT Protein Express LabChip filled with destain-gel, gel-dye and lower marker. Results was analyzed with LabChip GX Reviewer software.

CE-SDS-R: Reduced Capillary Electrophoresis Sodium Dodecyl Sulfate (CE-SDS-R) was performed based on a PerkinElmer Caliper automated electrophoresis that allows protein separation by size after reduction of sample and helps determine purity of heavy and light chains of the antibody. In the reduced microchip CE-SDS analysis, samples were diluted to 1 mg/mL with pure water. 2 μL of diluted samples were mixed with 7 μL of the sample denaturing solution (the mixture of sample buffer, 10% SDS solution and 1 M dithiothreitol as to reduction agent with the volume ratio of 100:10:4). The well-mixed samples were incubated at 70° C. for 10 minutes. 35 μL of water was added to each sample, vortexed for 10 seconds and centrifuged at 14000 rpm for 1 minute. 42 μL of the prepared samples were transferred to a 96-well plate, centrifuged at 4000 rpm for 15 minutes, placed onto a Labchip GXII's plate holder, separated and detected in the HT Protein Express LabChip filled with destain-gel, gel-dye and lower marker. Results was analyzed with LabChip GX Reviewer software.

Potency: Nerve growth factor (NGF) is a member of the neurotrophin family and is essential for the development and phenotypic maintenance of neurons in the peripheral nervous system and for the functional integrity of the cholinergic neurons in the central nervous system. This assay is an ELISA method for the binding analysis of IVX-01101 monoclonal antibody (mAb) to canine NGF. Samples, assay control (AC) and standard (STD) at appropriate dilutions are loaded onto to the antigen canine NGF protein coated 96-well high binding plate. After washing plate, Peroxidase-AffiniPure Rabbit Anti-Dog IgG (H+L) antibody added to the wells allowing interaction with IVX-01101 mAb captured during the previous step. After a finally washing, the TMB substrate solution is loaded into wells. TMB specifically reacts with the peroxide in the presence of peroxidase and produces a colorimetric signal that is proportional to the amount of IVX-01101 bound to the NGF-coated wells.

The first step of the ELISA method is to coat the 96-well plates with 100 μL/well of 3 μg/mL of Canine NGF protein solution. Seal plates with plate seal film and incubate the plate for 16-86 h at 2 to 8° C. Wash plate 6 times with approximately 300 μL/well of 0.05% PBST, Add 200 μL/well of blocking buffer (1% BSA/PBS) to the plate. Seal the plate with plate sealer, shake plate with plate shaker at 220 RPM, 25° C. for 1-3 h. then transfer 100 μL of diluted IVX-01101 antibody into separate wells in the 96-well plate. The standard curve is from 20000 ng/mL to 0 ng/mL. Incubate plate for 70±20 min on plate shaker at approximately 220 RPM at 25° C. Add 100 μL/well of Peroxidase-AffiniPure Rabbit Anti-Dog IgG (H+L) antibody solution to the plate. Seal the plate with plate sealer. Incubate plate for 70±20 min on plate shaker at approximately 220 RPM at 25° C. Lastly, add 100 μL/well of TMB (For KPL-5120-0047 kit, mix equal volume of TMB Peroxidase substrate and Peroxidase substrate solution B.) to all wells. Stop reaction after 10-20 min by adding 100 μL/well of 1 M HCll.

TMB used in the ELISA method specifically reacts with the peroxide in the presence of peroxidase and produces a colorimetric signal that is proportional to the amount of IVX-01101 bound to the wells. Dose response curves of IVX-01101 data according to a 4-parameter logistic (Auto-Estimate) regression model were completed using the SoftMax Pro software.

### Study Design

In this formulation development report, pH/buffer systems, excipients and surfactant strength were screened. The detailed study design is shown in Table 2.

TABLE 2
The study design of IVX-01101 formulation development study

| Study | Description |
|---|---|
| pH/buffer | Twelve pH/buffer types under stress conditions (40° C. for |
| screening | up to 4 weeks (W)) were investigated in the pH/buffer |
| study | screening study. The visual appearance, pH, protein |
| | concentration, SE-UPLC, iCIEF, DSC and CE-SDS-non- |
| | reduced and reduced were tested in this study. |
| Excipients/ | Different types of excipients (sucrose, trehalose dihydrate, |
| surfactant | sorbitol, L-arginine hydrochloride, sodium chloride, L- |
| strength | methionine, EDTA) and various concentration of PS80 |
| screening | (0.01%, 0.02%, 0.04% and 0.06%) were evaluated under |
| study | stress conditions (40° C. for up to 4 weeks, agitation at 300 |
| | rpm for 3 days (D), and freeze/thaw for up to 5 cycles |
| | (CYS). Visual appearance, pH, protein concentration, |
| | osmolality, SE-UPLC, CE-SDS-NR&R, iCIEF, MFI and |
| | potency testing were performed in this study. |

pH/Buffer Screening Study

Objective: The pH/buffer screening study aim was to select the optimal pH/buffer system to best stabilize the protein, and was conducted under stress conditions (40° C. incubation).

Sample Preparation: IVX-01101 drug substance (DS, Lot No.: SH23362110) was first buffer-exchanged into 9 buffers including 20 mM L-Glutamic acid/NaOH (pH 4.5, pH 5.0), 20 mM citric acid 1-hydrate/sodium citrate dehydrate buffer (pH4.5, pH5.0), 20 mM succinate acid/sodium succinate hexahydrate buffer (pH 5.0), 20 mM L-Histidine/L-Histidine monohydrochloride buffer (pH 6.5), and 20 mM phosphate buffer (pH 6.5, pH 7.0, pH 7.5), and then concentrated to the target protein concentration (30.0 mg/mL), respectively (Table 3). Each of the prepared samples was filtered with a 0.22 μm polyvinylidene fluoride (PVDF) filter and filled into 2 mL glass vials (1.0 mL per vial), then stoppered, capped and labeled immediately. All procedures were carried out in a bio-safety hood.

TABLE 3
Formulation candidates list of pH/buffer screening study

| Formulation No. | Buffer system | Target pH |
|---|---|---|
| Fb1 | 20 mM L-Glutamic acid/NaOH | 4.5 |
| Fb2 | | 5.0 |
| Fb3 | 20 mM citric acid 1-hydrate/sodium | 4.5 |
| Fb4 | citrate dehydrate | 5.0 |
| Fb5 | 20 mM succinate acid/sodium | 5.0 |
| | succinate hexahydrate buffer | |
| Fb6 | 20 mM L-Histidine/L-Histidine | 6.5 |
| | monohydrochloride | |
| Fb7 | 20 mM Phosphate buffer | 6.5 |
| Fb8 | | 7.0 |
| Fb9 | | 7.5 |

Study Parameters: Table 4 shows the sampling and testing plan for the pH/buffer screening study. Samples were stored at 40° C. for up to 4 weeks and pulled at each time point. Testing included appearance, pH, protein concentration, SE-UPLC, iCIEF, CE-SDS-NR&R and DSC for in this study.

TABLE 4
Sampling and testing plan of pH/buffer screening study

| | | 40° C. | | |
|---|---|---|---|---|
| Formulation No. | T0 | 1 W | 2 W | 4 W |
| Fb1-Fb9 | X, Y | X | X | X |

X = Appearance, protein conc., SE-UPLC, iCIEF, CE-SDS-NR & R

### Results

Table 5 shows the data summary of DSC results from the pH/buffer screening study. The Tₒₙₛₑₜ values were higher than 54.9° C. in all formulations, indicating that the 40° C. thermal condition was suitable for the stress study.

TABLE 5
pH/Buffer screening study: DSC

| Formulation | | | |
|---|---|---|---|
| No. | Tₒₙₛₑₜ (° C.) | Tₘ1 (° C.) | Tₘ2 (° C.) |
| Fb1 | 54.9 | 67.0 | 74.8 |
| Fb2 | 58.3 | 70.2 | 77.2 |
| Fb3 | 55.3 | 67.6 | 75.7 |
| Fb4 | 58.2 | 69.9 | 76.8 |
| Fb5 | 56.8 | 69.1 | 76.1 |
| Fb6 | 58.9 | 71.1 | 79.7 |
| Fb7 | 60.4 | 71.8 | 79.6 |
| Fb8 | 60.4 | 71.6 | 79.1 |
| Fb9 | 58.7 | 71.6 | 78.7 |

Appearance, pH, and protein concentration: Table 6 provides the data from the appearance testing for the pH/buffer screening study.

According to the T0 appearance result, all formulations were colorless, slight opalescent liquid and not essentially free of visible particles except Fb1 (20 mM L-Glutamic acid/NaOH, pH 4.5) and Fb7 (20 mM PB buffer, pH 6.5) showed free of visible particles. And visible particles were observed in all formulations after 1 W, 2 W and 4 W incubation under 40° C. For the buffer at pH 5.0, Fb2 (20 mM L-Glutamic acid/NaOH), Fb4 (20 mM citric acid 1-hydrate/sodium citrate dehydrate) and Fb5 (20 mM succinate acid/sodium succinate hexahydrate buffer), amount of visible particles increased obviously.

TABLE 6
pH/Buffer screening study: appearance

| | Appearance | | | |
|---|---|---|---|---|
| Formulation | | 40° C. | | |
| No. | T0 | 1 W | 2 W | 4 W |
| Fb1 | C, SOL, | C, SOL, | C, SOL, NEFP | C, SOL, NEFP |
| | FP | NEFP | | |
| Fb2 | C, SOL, | C, SOL, | C, SOL, NEFP | C, HOL, NEFP |
| | NEFP | NEFP | | |
| Fb3 | C, SOL, | C, SOL, | C, SOL, NEFP | C, SOL, NEFP |
| | NEFP | NEFP | | |
| Fb4 | C, SOL, | C, SOL, | C, SOL, NEFP | C, HOL, NEFP |
| | NEFP | NEFP | | |
| Fb5 | C, SOL, | C, HOL, | C, HOL, NEFP | C, HOL, NEFP |
| | NEFP | NEFP | | |
| Fb6 | C, SOL, | C, SOL, | C, SOL, NEFP | C, SOL, NEFP |
| | NEFP | NEFP | | |
| Fb7 | C, SOL, | C, SOL, | C, SOL, NEFP | C, SOL, NEFP |
| | FP | NEFP | | |
| Fb8 | C, SOL, | C, SOL, | C, SOL, NEFP | C, SOL, NEFP |
| | NEFP | NEFP | | |
| Fb9 | C, SOL, | C, SOL, | C, SOL, NEFP | C, SOL, NEFP |
| | NEFP | NEFP | | |

Abbreviation:
C = colorless,
SOL = slightly opalescent liquid,
HOL = heavy opalescence liquid,
FP = free of visible particles,
NEFP (not essentially free of visible particles, >5 particles observed).

Table 7 provides the data from the pH and concentration testing for the pH1/buffer screening study.

For pH results, the pH of all formulations were around the target value, except obvious pH shift from 5.0 to 5.4 was measured at Fb2 (20 mM L-Glutamic acid/NaOH) after buffer exchange. For concentration results, all formulations were around the target value (30.0 mg/mL). No obvious changes were observed in all formulations after 4 W incubation under 40° C.

TABLE 7
pH/Buffer screening study: pH and protein concentration

| | | Protein conc. (mg/mL) | | | |
|---|---|---|---|---|---|
| Formulation | pH | | 40° C. | | |
| No. | T0 | T0 | 1 W | 2 W | 4 W |
| Fb1 | 4.7 | 29.0 | 29.2 | 29.3 | 29.3 |
| Fb2 | 5.4 | 29.4 | 29.4 | 29.4 | 29.5 |
| Fb3 | 4.6 | 28.9 | 29.0 | 29.2 | 29.1 |
| Fb4 | 5.2 | 29.1 | 29.2 | 29.5 | 29.8 |
| Fb5 | 5.1 | 28.9 | 29.2 | 29.8 | 30.4 |
| Fb6 | 6.3 | 29.5 | 29.1 | 29.4 | 29.4 |
| Fb7 | 6.5 | 29.1 | 29.2 | 29.4 | 29.4 |
| Fb8 | 6.9 | 29.3 | 29.3 | 29.5 | 29.6 |
| Fb9 | 7.4 | 29.4 | 29.3 | 29.5 | 29.4 |

SE-UPLC: Table 8 provides the data from the SE-UPLC testing for the pH/buffer screening study.

SEC monomer decreases were observed in all formulations. Fb1 (20 mM L-Glutamic acid/NaOH at pH 4.5, 12.3% decreased) and Fb3 (20 mM citric acid 1-hydrate/sodium citrate dehydrate at pH 4.5, 11.3% decreased) had the highest decrease of SEC monomer after 4 W incubation at 40° C. Fb6 (20 mM L-Histidine/L-Histidine monohydrochloride buffer at pH 6.5, 3.8% decreased), Fb7 (20 mM PB buffer at pH 6.5, 3.6% decreased) and Fb8 (20 mM PB buffer at pH 7.0, 3.9% decreased) had the lowest decrease of SEC monomer after 4 W incubation at 40° C.

TABLE 8
pH/Buffer screening study: SE-UPLC

| | SE-UPLC | | | | | | | | | | | |
|---|---|---|---|---|---|---|---|---|---|---|---|---|
| | Monomer (%) | | | | HMW (%) | | | | LMW (%) | | | |
| | | 40° C. | | | | 40º C. | | | | 40° C. | | |
| Formulation | | 1 | 2 | 4 | | 1 | 2 | 4 | | 1 | 2 | 4 |
| No. | T0 | W | W | W | T0 | W | W | W | T0 | W | W | W |
| Fb1 | 99.6 | 94.1 | 91.2 | 87.6 | ND | 4.7 | 6.8 | 8.5 | 0.3 | 1.3 | 2.0 | 3.9 |
| Fb2 | 99.0 | 96.8 | 95.9 | 94.3 | 0.6 | 2.5 | 3.2 | 4.1 | 0.3 | 0.6 | 0.9 | 1.6 |
| Fb3 | 99.2 | 94.3 | 91.5 | 87.9 | 0.4 | 4.6 | 6.7 | 8.7 | 0.3 | 1.2 | 1.8 | 3.4 |
| Fb4 | 99.0 | 96.5 | 95.3 | 92.6 | 0.7 | 2.8 | 3.6 | 5.4 | 0.3 | 0.7 | 1.1 | 2.0 |
| Fb5 | 99.1 | 96.3 | 95.3 | 93.5 | 0.3 | 2.8 | 3.3 | 3.9 | 0.6 | 0.9 | 1.4 | 2.6 |
| Fb6 | 99.7 | 98.1 | 97.4 | 95.9 | ND | 1.4 | 1.9 | 2.9 | 0.3 | 0.5 | 0.7 | 1.1 |
| Fb7 | 99.0 | 97.1 | 96.5 | 95.4 | 0.7 | 2.3 | 2.8 | 3.6 | 0.3 | 0.6 | 0.7 | 0.9 |
| Fb8 | 99.0 | 96.9 | 96.3 | 95.1 | 0.7 | 2.2 | 2.7 | 3.6 | 0.3 | 0.8 | 1.0 | 1.3 |
| Fb9 | 99.0 | 96.9 | 96.1 | 94.4 | 0.7 | 2.1 | 2.8 | 4.1 | 0.3 | 1.0 | 1.1 | 1.5 |

Abbreviation: HMW, high molecular weight; LMW, low molecular weight; ND, not detected.

iCIEF: Table 9 provides the data from the iCIEF testing for the pH/buffer screening study.

Changes in iCIEF peak were observed on all formulations after 4 W incubation 40° C. Fb6 (20 mM L-Histidine/L-Histidine monohydrochloride buffer, pH 6.5) had the smallest decrease on iCIEF after incubation at 40° C. for 4 W.

TABLE 9
pH/Buffer screening study: iCIEF

| | iCIEF | | | | | | | | | | | | | | | | | | | |
|---|---|---|---|---|---|---|---|---|---|---|---|---|---|---|---|---|---|---|---|---|
| For- | GA (%) | | | | P1 (%) | | | | P2 (%) | | | | P3 (%) | | | | GB (%) | | | |
| mulation | | 40° C. | | | | 40° C. | | | | 40° C. | | | | 40° C. | | | | 40° C. | | |
| No. | T0 | 1 W | 2 W | 4 W | T0 | 1 W | 2 W | 4 W | T0 | 1 W | 2 W | 4 W | T0 | 1 W | 2 W | 4 W | T0 | 1 W | 2 W | 4 W |
| Fb1 | 14.9 | 9.2 | 8.0 | 6.4 | 25.1 | 16.8 | 12.9 | 7.7 | 31.2 | 27.5 | 24.1 | 21.1 | 23.9 | 26.6 | 23.7 | 22.3 | 5.0 | 19.9 | 31.4 | 42.5 |
| Fb2 | 15.4 | 11.4 | 10.5 | 11.8 | 25.5 | 19.7 | 18.7 | 12.4 | 30.2 | 31.2 | 27.3 | 29.9 | 23.9 | 23.8 | 24.5 | 22.2 | 5.0 | 13.2 | 17.2 | 23.0 |
| Fb3 | 15.3 | 8.0 | 7.2 | 6.9 | 24.6 | 17.8 | 13.4 | 8.4 | 29.4 | 27.3 | 23.0 | 20.6 | 23.6 | 26.1 | 25.1 | 22.7 | 7.1 | 20.0 | 30.3 | 39.0 |
| Fb4 | 15.6 | 10.6 | 8.9 | 9.9 | 25.2 | 18.8 | 17.1 | 9.9 | 30.8 | 30.2 | 25.5 | 25.7 | 23.3 | 24.2 | 24.6 | 25.1 | 5.2 | 16.3 | 22.9 | 28.4 |
| Fb5 | 15.3 | 9.7 | 9.6 | 10.4 | 25.7 | 19.6 | 17.9 | 12.1 | 29.9 | 28.2 | 23.0 | 22.1 | 23.6 | 26.0 | 24.5 | 23.2 | 5.5 | 16.6 | 24.2 | 30.6 |
| Fb6 | 15.6 | 15.6 | 18.9 | 26.3 | 25.6 | 27.7 | 28.1 | 30.4 | 31.2 | 29.5 | 28.2 | 24.8 | 22.0 | 18.0 | 16.4 | 10.7 | 5.6 | 9.3 | 7.9 | 7.9 |
| Fb7 | 15.9 | 20.4 | 29.1 | 13.5 | 25.7 | 32.4 | 34.6 | 26.1 | 30.4 | 29.5 | 22.9 | 35.2 | 22.7 | 10.8 | 7.3 | 16.0 | 5.4 | 6.1 | 6.1 | 5.2 |
| Fb8 | 16.7 | 12.4 | 13.8 | 26.3 | 25.6 | 17.9 | 28.8 | 28.6 | 29.7 | 40.9 | 43.0 | 33.7 | 21.3 | 19.5 | 8.3 | 4.8 | 6.6 | 2.0 | 1.7 | 3.8 |
| Fb9 | 17.2 | 14.5 | 22.9 | 36.4 | 26.9 | 25.4 | 30.1 | 29.5 | 30.3 | 47.1 | 36.5 | 24.9 | 19.9 | 8.0 | 5.6 | 3.1 | 5.7 | 2.4 | 2.4 | 2.2 |

CE-SDS-NR & R: Table 10 provides the data from the CE-SDS-NR and CE-SDS-R testing for the pH/buffer screening study.

For CE-SDS-NR results, a 7.0%-14.7% decrease in CE-SDS-NR purity were observed for 9 formulations after 4 W incubation at 40° C. Fb1 (20 mM L-Glutamic acid/NaOH at pH 4.5, 14.7% decreased) and Fb3 (20 mM citric acid 1-hydrate/sodium citrate dehydrate at pH 4.5, 14.2% decreased) had the most purity decreases among all formulations. Fb5 (20 mM succinate acid/sodium succinate hexahydrate buffer at pH 6.5, 7.6% decreased) and Fb7 (20 mM PB buffer at pH 6.5, 7.0% decreased) had minimal purity decreases among all formulations after incubation at 40° C. for 1 W or 2 W.

For CE-SDS-R results, 1.2%-6.0% decreases in CE-SDS-R purity were observed for 9 formulations after 4 W incubation at 40° C. Fb9 (20 mM PB buffer at pH 6.5, 6.0% decreased) had the most purity decreases among all formulations. Fb7 (20 mM PB buffer at pH 6.5, 1.2% decreased) had the minimal purity decreases.

TABLE 10
pH/Buffer screening study: CE-SDS-NR & R

| For- | CE-SDS-NR (Purity %) | | | | CE-SDS-R (Purity %) | | | |
|---|---|---|---|---|---|---|---|---|
| mulation | | 40° C. | | | | 40° C. | | |
| No. | T0 | 1 W | 2 W | 4 W | T0 | 1 W | 2 W | 4 W |
| Fb1 | 96.8 | 94.6 | 90.4 | 82.1 | 99.0 | 98.2 | 96.4 | 95.0 |
| Fb2 | 96.6 | 95.2 | 92.5 | 87.3 | 99.5 | 98.7 | 98.0 | 97.9 |
| Fb3 | 96.4 | 94.5 | 90.3 | 82.2 | 99.4 | 98.3 | 95.9 | 95.9 |
| Fb4 | 96.7 | 95.3 | 93.3 | 87.6 | 99.5 | 98.8 | 97.0 | 97.0) |
| Fb5 | 96.6 | 95.1 | 92.8 | 89.0 | 99.5 | 98.8 | 95.3 | 97.0 |
| Fb6 | 96.6 | 95.5 | 94.3 | 88.5 | 99.4 | 98.7 | 97.9 | 97.5 |
| Fb7 | 96.6 | 95.8 | 94.2 | 89.6 | 99.4 | 98.8 | 97.6 | 98.2 |
| Fb8 | 96.6 | 95.2 | 93.6 | 88.0 | 99.5 | 98.7 | 97.9 | 97.3 |
| Fb9 | 96.5 | 94.5 | 91.9 | 83.9 | 99.4 | 98.3 | 96.8 | 93.4 |

The pH/buffer screening study investigated nine pH/buffer types under stress conditions (40° C. for up to 4 weeks), and the results showed stability of IVX-01101 was closely related with both pH and buffer type. Based on all of the tests performed in this study, the protein exhibited poor protein stability at pH 4.5 and pH 5.0, and exhibited better protein stability at pH 6.5.

From the appearance results, Fb1 (20 mM L-Glutamic acid/NaOH buffer at pH 4.5) and Fb7 (20 mM PB buffer at pH 6.5) were free of visible particles at T0. The number of visible particles were visibly increased in Fb2 (20 mM L-Glutamic acid/NaOH, pH 5.0), Fb4 (20 mM citric acid 1-hydrate/sodium citrate dehydrate, pH 5.0) and Fb5 (20 mM succinate acid/sodium succinate hexahydrate buffer, pH 5.0) accompanied by a heavier opalescence level. No substantial changes were observed in protein concentration with any of the tested buffers.

The SE-UPLC results indicated that Fb6 (20 mM L-Histidine/L-Histidine monohydrochloride buffer, pH 6.5), Fb7 (20 mM PB buffer, pH 6.5) and Fb8 (20 mM PB buffer, pH 6.5) resulted in highest purity when the protein was stored at 40° C. for 4 weeks.

The iCIEF results indicated Fb6 (20 mM L-Histidine/L-Histidine monohydrochloride buffer, pH 6.5) had the smallest change on iCIEF.

The CE-SDS-NR&R results showed less substantial difference in purity when IVX-01101 was formulated in Fb6 20 mM L-Histidine/L-Histidine monohydrochloride buffer, pH 6.5) and Fb7 (20 mM PB buffer, pH 6.5).

In conclusion, a 20 mM L-Histidine/L-Histidine monohydrochloride buffer was selected for use with the IVX-01101 protein and the optimal pH was determined to be 6.5.

### Example 2: IVX-01101 Excipients and Surfactant Strength Screening Study

Objective: The excipients and surfactant strength screening study served to screen the optimal excipients and surfactant strengths that best stabilized the exemplary protein. This study was conducted under stress conditions (40° C. incubation, multiple freeze and thaw cycles, agitation).

Sample Preparation: For Fe1 to Fe11, the DS (Lot No.: SH23362110) was initially buffer-exchanged into the pre-prepared 20 mM L-Histidine/L-Histidine monohydrochloride buffer (pH 6.5). Then stock solutions of 40% (w/w) sucrose, 44% (w/w) trehalose 2H₂O dihydrate, 30% (w/w) sorbitol, 700 mM sodium chloride, 700 mM L-arginine hydrochloride, 100 mM L-methionine, 10 mM EDTA, and 5% (w/w) polysorbate 80 (PS80) were prepared. The amounts of DS, excipient stock solutions and surfactant stock solution were calculated, weighed and well-mixed based on the formulation recipes for Fe1-Fe11. All formulation samples listed in Table 11 were eventually filtered with 0.22 μm PVDF filter, filled into 6 mL glass vials (3.0 mL per vial), stoppered, capped and labeled immediately.

TABLE 11
Formulation candidates with list of excipients and surfactant
strength screening study

| Formulation | Buffer System, pH, | |
|---|---|---|
| No. | Protein Conc. | Excipients/Surfactant |
| Fe1 | 20 mM L-Histidine/ | 8% sucrose, 0.02% PS80 |
| Fe2 | L-Histidine | 8.8% trehalose•2H₂O, |
| | monohydrochloride | 0.02% PS80 |
| Fe3 | buffer, pH 6.5, | 4.5% sorbitol, 0.02% PS80 |
| Fe4 | 30.0 mg/mL | 140 mM Arg-HCl, 0.02% PS80 |
| Fe5 | | 140 mM NaCl, 0.02% PS80 |
| Fe6 | | 8% sucrose |
| Fe7 | | 8% sucrose, 0.01% PS80 |
| Fe8 | | 8% sucrose, 0.04% PS80 |
| Fe9 | | 8% sucrose, 0.06% PS80 |
| Fe10 | | 8% sucrose, 10 mM |
| | | L-Methionine 0.02% PS80 |
| Fe11 | | 8% sucrose, 10 mM Methionine, |
| | | 0.05 mM EDTA, 0.02% PS80 |

Note:
all the % in the Table 11 mean % (w/v).

Study Parameters: Table 12 shows the sampling and testing plan for the excipients and surfactant strength screening study. For freeze and thaw (FT), samples were completely frozen at −70° C. and thawed at room temperature (RT). For thermal stress, samples were stored at 40° C. for up to 4 weeks. For agitation stress, samples were agitated at 300 rpm, 25° C. for up to 3 days. Testing included appearance, pH, protein concentration, osmolality, SE-UPLC, CE-SDS-NR&R, iCIEF, MFI and potency for this study.

TABLE 12
Sampling and testing plan of excipients with surfactant strength
screening study

| Formulation | | | | Sampling points | |
|---|---|---|---|---|---|
| No. | Attribute | Condition | T0 | and assay | |
| Fe1-Fe11 | Thermal | 40° C. | X, Y, Z, | 2 W | 4 W |
| | | | S, P | X, Y | X, Y, S, P |
| | Freeze/Thaw | −70° C. to RT | | 1 D | 3 D |
| | | | | X | X, Y, S |
| | Agitation | 300 rpm, | | 3 CYS | 5 CYS |
| | | 25° C. | | X | X, Y, S |

X = Appearance, protein conc., SE-UPLC
Y = CE-SDS-NR&R, iCIEF
Z = Osmolality, pH
S = Sub-visible particles (MFI)
P = Potency (Only for the selected formulation)

### Results

Appearance, pH, and protein conc.: Table 13 provides the data from the appearance testing for the excipients in the surfactant strength screening study.

Except for Fe1, Fe4 to Fe9, all formulations maintained good appearance with clear, slight opalescence and no visible particles in this study. For formulations with sucrose, Fe1, Fe6 to Fe9 samples became slightly yellow after 4 W incubation at 40° C. For Fe4, Fe5, and Fe6, deeper opalescence was observed after agitation. For Fe6, visible particles were observed after agitation, freeze/thaw and thermal stress.

TABLE 13
Excipients and surfactant strength screening study: appearance

| | Appearance | | | | | | |
|---|---|---|---|---|---|---|---|
| Formulation | | Agitation | | FT | | 40° C. | |
| No. | T0 | 1 D | 3 D | 3 CYS | 5 CYS | 2 W | 4 W |
| Fe1 | C, SOL, | C, SOL, F | C, SOL, F | C, SOL, F | C, SOL, F | C, SOL, F | SY, SOL, |
| | FP | P | P | P | P | P | FP |
| Fe2 | C, SOL, | C, SOL, F | C, SOL, F | C, SOL, F | C, SOL, F | C, SOL, F | C, SOL, F |
| | FP | P | P | P | P | P | P |
| Fe3 | C, SOL, | C, SOL, F | C, SOL, F | C, SOL, F | C, SOL, F | C, SOL, F | C, SOL, F |
| | FP | P | P | P | P | P | P |
| Fe4 | C, SOL, | C, SOL+, | C, SOL+, | C, SOL, F | C, SOL, F | C, SOL, F | C, SOL, F |
| | FP | FP | FP | P | P | P | P |
| Fe5 | C, SOL, | C, SOL+, | C, SOL+, | C, SOL, F | C, SOL, F | C, SOL, F | C, SOL, F |
| | FP | FP | FP | P | P | P | P |
| Fe6 | C, SOL, | C, SOL+, | C, SOL+, | C, SOL, N | C, SOL, N | C, SOL, N | SY, SOL, |
| | FP | NEFP | NEFP | EFP | EFP | EFP | NEFP |
| Fe7 | C, SOL, | C, SOL, F | C, SOL, F | C, SOL, F | C, SOL, F | C, SOL, F | SY, SOL, |
| | FP | P | P | P | P | P | FP |
| Fe8 | C, SOL, | C, SOL, F | C, SOL, F | C, SOL, F | C, SOL, F | C, SOL, F | SY, SOL, |
| | FP | P | P | P | P | P | FP |
| Fe9 | C, SOL, | C, SOL, F | C, SOL, F | C, SOL, F | C, SOL, F | C, SOL, F | SY, SOL, |
| | FP | P | P | P | P | P | FP |
| Fe10 | C, SOL, | C, SOL, F | C, SOL, F | C, SOL, F | C, SOL, F | C, SOL, F | C, SOL, F |
| | FP | P | P | P | P | P | P |
| Fe11 | C, SOL, | C, SOL, F | C, SOL, F | C, SOL, F | C, SOL, F | C, SOL, F | C, SOL, F |
| | FP | P | P | P | P | P | P |

Abbreviation: C = colorless; SY = slight yellow; SOL = slightly opalescent liquid; SOL += slightly opalescent liquid with increased opalescence; FP = free of visible particles; NEFP = not essentially of visible particles (>5 particles observed).

Table 14 provides the data from the pH and protein concentration testing for the excipients in the surfactant strength screening study.

The pH values and protein concentration of all formulations were around the target value with a pH 6.5, 30.0 mg/mL and no obvious changes were observed after agitation, freeze/thaw and thermal stress.

TABLE 14
Excipients and surfactant strength screening study:
pH and concentration

| | | Protein conc. (mg/mL) | | | | | | |
|---|---|---|---|---|---|---|---|---|
| For- | | | | | FT | | | |
| mulation | pH | | Agitation | | 3 | 5 | 40° C. | |
| No. | T0 | T0 | 1 D | 3 D | CYS | CYS | 2 W | 4 W |
| Fe1 | 6.4 | 29.6 | 29.5 | 29.2 | 29.8 | 29.2 | 29.2 | 28.8 |
| Fe2 | 6.4 | 29.8 | 29.5 | 29.6 | 29.4 | 29.3 | 29.3 | 29.0 |
| Fe3 | 6.4 | 28.8 | 28.7 | 29.2 | 29.1 | 29.3 | 29.7 | 28.8 |
| Fe4 | 6.4 | 29.3 | 28.8 | 29.5 | 29.1 | 29.3 | 29.0 | 28.7 |
| Fe5 | 6.5 | 29.5 | 29.4 | 29.8 | 29.5 | 29.0 | 29.6 | 28.9 |
| Fe6 | 6.4 | 29.5 | 29.8 | 30.9 | 29.9 | 30.4 | 29.6 | 31.1 |
| Fe7 | 6.4 | 29.4 | 29.3 | 30.3 | 29.6 | 29.3 | 29.0 | 29.1 |
| Fe8 | 6.4 | 28.7 | 28.6 | 29.6 | 29.2 | 29.2 | 28.9 | 28.9 |
| Fe9 | 6.4 | 29.2 | 29.0 | 29.8 | 29.3 | 29.1 | 29.0 | 29.1 |
| Fe10 | 6.4 | 29.0 | 29.2 | 29.7 | 29.0 | 29.2 | 29.0 | 29.0 |
| Fe11 | 6.4 | 29.3 | 29.2 | 29.4 | 29.4 | 29.4 | 28.9 | 28.7 |

Sub-visible particles (MFI): Table 15 provides the data from the sub-visible parties (MFI) testing for the excipients and surfactant strength screening study. Fe4 (with Arg-HCl) had significant increases in sub-visible particles after freeze/thaw for 5 cycles. Increased sub-visible particles were observed in Fe6 (only with sucrose but no surfactant added) under agitation for 3 days, freeze/thaw for 5 cycles and incubation at 40 C for 4 weeks. Other formulations (Fe1 to Fe3, Fe5, Fe7 to Fe11) had no obvious changes in sub-visible particles under all stress conditions.

TABLE 15
Excipients and surfactant strength screening study:
sub-visible particles (MFI)

| | Sub-visible particles counts | | | |
|---|---|---|---|---|
| | (#/mL, ≥2 μm/≥10 μm/≥25 μm) | | | |
| Formulation | | Agitation | FT | 40° C. |
| No. | T0 | 3 D | 5 CYS | 4 W |
| Fe1 | 507/22/0 | 189/10/4 | 631/12/0 | 993/19/4 |
| Fe2 | 454/15/2 | 713/7/0 | 656/23/5 | 562/32/2 |
| Fe3 | 759/33/2 | 420/17/0 | 759/9/0 | 1183/68/2 |
| Fe4 | 656/20/0 | 446/4/0 | 4785/344/5 | 1026/58/2 |
| Fe5 | 960/50/2 | 656/12/0 | 2357/55/0 | 1334/37/0 |
| Fe6 | 4590/132/4 | 145453/15953/ | 7351/389/33 | Out of detection |
| | | 3576 | | range |
| Fe7 | 359/9/0 | 710/4/0 | 611/33/2 | 1412/74/0 |
| Fe8 | 443/9/0 | 598/4/0 | 931/45/10 | 1507/45/0 |
| Fe9 | 295/15/0 | 805/0/0 | 731/7/2 | 1078/19/2 |
| Fe10 | 552/15/2 | 733/4/0 | 721/2/0 | 1731/68/0 |
| Fe11 | 531/15/0 | 508/5/0 | 631/5/0 | 770/33/10 |

SE-UPLC: Table 16 provides the data from SE-UPLC testing for the excipients and surfactant strength screening study.

Compared to T0 samples, no obvious changes in the SEC monomer were observed in formulations under 3 days of agitation and 5 cycles of freeze/thaw stress, except for the Fe6 formulation (only with sucrose but no surfactant added) where a 0.600 decrease in the SEC monomer was observed after 5 cycles of freeze/thaw stress.

After being incubated at 40° C. for 4 weeks, decreases ranging from 1.9-7.3% in the SEC monomer were observed in all formulations. Comparing the excipient used, results demonstrated that Fe1-Fe5, Fe2 (with trehalose) and Fe3 (with sorbitol) formulations performed better than other formulations with 0.02% (w/v) PS80, as the decline in the SEC monomer was less than 3.0%. Evaluating the surfactant amount by comparing Fe1 and Fe6 to Fe9, samples with PS80 less than 0.02% (w/v) showed higher SEC monomer than other samples, with SEC monomer concentration decreasing with increased concentration of PS80. Adding 10 mM L-methionine and 0.05 mM EDTA to the Fe11 formulation was found to prevent high molecule weight (HMW) species effectively. Overall, Fe2, Fe3 and Fe11 performed best amongst among the 11 formulations tested.

TABLE 16
Excipients and surfactant strength screening study: SE-UPLC (HMW: high molecular
weight; LMW, low molecular weight).

| | SE-UPLC | | | | | | | | | | | | | | | | | | | | |
|---|---|---|---|---|---|---|---|---|---|---|---|---|---|---|---|---|---|---|---|---|---|
| For- | Monomer (%) | | | | | | | HMW (%) | | | | | | | LMW (%) | | | | | | |
| mu- | | Agi- | | FT | | | | | Agi- | | FT | | | | | Agi- | | FT | | | |
| lation | | tation | | 3 | 5 | 40° C. | | | tation | | 3 | 5 | 40° C. | | | tation | | 3 | 5 | 40° C. | |
| No. | T0 | 1 D | 3 D | CYS | CYS | 2 W | 4 W | T0 | 1 D | 3 D | CYS | CYS | 2 W | 4 W | T0 | 1 D | 3 D | CYS | CYS | 2 W | 4 W |
| Fe1 | 98.9 | 98.8 | 98.8 | 98.9 | 98.8 | 96.0 | 94.7 | 0.4 | 0.5 | 0.6 | 0.5 | 0.5 | 2.9 | 4.1 | 0.7 | 0.7 | 0.6 | 0.7 | 0.7 | 1.1 | 1.3 |
| Fe2 | 98.7 | 98.7 | 98.7 | 98.7 | 98.7 | 97.1 | 95.9 | 0.6 | 0.7 | 0.7 | 0.7 | 0.7 | 1.9 | 2.9 | 0.7 | 0.7 | 0.6 | 0.7 | 0.7 | 1.0 | 1.2 |
| Fe3 | 98.7 | 98.7 | 98.6 | 98.6 | 98.6 | 97.0 | 95.8 | 0.6 | 0.7 | 0.7 | 0.7 | 0.7 | 2.0 | 3.0 | 0.7 | 0.7 | 0.6 | 0.7 | 0.7 | 1.0 | 1.2 |
| Fe4 | 98.7 | 98.6 | 98.5 | 98.6 | 98.5 | 96.7 | 95.4 | 0.7 | 0.7 | 0.7 | 0.7 | 0.7 | 2.1 | 3.4 | 0.7 | 0.7 | 0.7 | 0.7 | 0.7 | 1.2 | 1.2 |
| Fe5 | 98.6 | 98.5 | 98.4 | 98.4 | 98.9 | 96.5 | 95.3 | 0.8 | 0.9 | 0.9 | 0.9 | 0.4 | 2.4 | 3.6 | 0.7 | 0.7 | 0.7 | 0.7 | 0.7 | 1.1 | 1.1 |
| Fe6 | 99.0 | 99.0 | 98.9 | 98.9 | 98.4 | 97.4 | 94.1 | 0.4 | 0.4 | 0.4 | 0.4 | 0.9 | 1.7 | 3.6 | 0.6 | 0.6 | 0.6 | 0.7 | 0.7 | 0.9 | 2.3 |
| Fe7 | 98.9 | 98.9 | 98.8 | 98.9 | 98.9 | 96.7 | 95.5 | 0.4 | 0.5 | 0.5 | 0.4 | 0.5 | 2.3 | 3.3 | 0.7 | 0.7 | 0.7 | 0.7 | 0.7 | 1.0 | 1.2 |
| Fe8 | 98.9 | 98.8 | 98.8 | 98.8 | 98.8 | 94.8 | 93.1 | 0.4 | 0.6 | 0.6 | 0.5 | 0.5 | 3.9 | 5.6 | 0.7 | 0.6 | 0.7 | 0.7 | 0.7 | 1.3 | 1.3 |
| Fe9 | 98.9 | 98.8 | 98.7 | 98.9 | 98.8 | 93.7 | 91.6 | 0.4 | 0.5 | 0.6 | 0.5 | 0.5 | 4.9 | 7.0 | 0.7 | 0.7 | 0.7 | 0.6 | 0.7 | 1.4 | 1.4 |
| Fe10 | 98.9 | 98.9 | 98.9 | 98.9 | 98.9 | 97.0 | 95.9 | 0.4 | 0.5 | 0.5 | 0.4 | 0.4 | 2.0 | 2.9 | 0.7 | 0.7 | 0.6 | 0.7 | 0.7 | 1.0 | 1.2 |
| Fe11 | 98.9 | 98.9 | 98.9 | 98.9 | 98.9 | 97.8 | 97.0 | 0.4 | 0.4 | 0.5 | 0.4 | 0.4 | 1.4 | 2.0 | 0.7 | 0.7 | 0.6 | 0.7 | 0.7 | 0.8 | 1.0 |

iCIEF: Table 17 provides the data from iCIEF testing for the excipients and surfactant strength screening study.

Comparing with T0 samples, no obvious changes of iCIEF main peak were observed in 11 formulations under 3 days of agitation and 5 cycles of freeze/thaw stress.

After being incubated at 40° C. for 4 weeks, decreases of the iCIEF peak were observed in all formulations, although Fe2, Fe3 and F11 performed the best amongst the 11 formulations.

TABLE 17
Excipients and surfactant strength screening study: iCIEF

| | iCIEF | | | | | | | | | | | | | | |
|---|---|---|---|---|---|---|---|---|---|---|---|---|---|---|---|
| For- | P1 (%) | | | | | P2 (%) | | | | | P3 (%) | | | | |
| mula- | | | FT | | | | | FT | | | | | FT | | |
| tion | | A | 5CY | 40° C. | | | A | 5CY | 40° C. | | | A | 5CY | 40° C. | |
| No. | T0 | 3D | S | 2 W | 4 W | T0 | 3D | S | 2 W | 4 W | T0 | 3D | S | 2 W | 4 W |
| Fb1 | 23.8 | 24.0 | 24.5 | 29.3 | 29.1 | 32.5 | 32.8 | 33.5 | 24.6 | 21.8 | 22.0 | 21.7 | 22.9 | 17.8 | 10.1 |
| Fb2 | 24.4 | 24.0 | 24.4 | 29.3 | 29.5 | 32.4 | 32.9 | 33.3 | 25.7 | 22.8 | 22.1 | 22.5 | 22.7 | 17.3 | 11.9 |
| Fb3 | 24.8 | 24.6 | 24.3 | 29.2 | 30.8 | 32.6 | 32.5 | 33.2 | 26.8 | 22.0 | 22.2 | 22.1 | 22.8 | 19.1 | 11.3 |
| Fb4 | 24.6 | 25.1 | 25 | 37.1 | 36.8 | 32.8 | 33.4 | 33.2 | 23.3 | 16.9 | 22.2 | 20.2 | 21.5 | 10.6 | 6.4 |
| Fb5 | 24.1 | 24.7 | 24.8 | 33.9 | 34.8 | 32.2 | 32.3 | 32.9 | 24.5 | 18.1 | 22.8 | 22.2 | 22.8 | 14.0 | 8.5 |
| Fb6 | 24.6 | 24.0 | 24.3 | 28.6 | 12.5 | 31.6 | 32.6 | 33.1 | 26.9 | 25.8 | 22.9 | 21.7 | 22.6 | 20.9 | 25.6 |
| Fb7 | 24.4 | 24.5 | 24.2 | 29.3 | 29.8 | 32.4 | 32.4 | 32.4 | 25.1 | 20.6 | 21.5 | 22.2 | 23.0 | 19.8 | 12.6 |
| Fb8 | 23.8 | 24.8 | 24.4 | 32.6 | 27.7 | 32.4 | 32.9 | 32.9 | 24.2 | 23.4 | 23.1 | 22.4 | 22.6 | 16.0 | 10.5 |
| Fb9 | 23.8 | 25.1 | 25.1 | 28.3 | 27.8 | 32.4 | 31.8 | 33.5 | 23.5 | 22.2 | 21.9 | 22.9 | 23.2 | 14.3 | 7.4 |
| Fb10 | 24.0 | 24.9 | 24.2 | 24.8 | 29.8 | 31.9 | 33 | 33.3 | 30.1 | 20.7 | 23.3 | 22.1 | 22.9 | 18.4 | 11.0 |
| Fb11 | 24.3 | 24.0 | 25.3 | 28.5 | 29.9 | 32.3 | 33.6 | 33.1 | 26.6 | 22.8 | 22.1 | 21.9 | 23.0 | 18.6 | 13.1 |

Excipients and surfactant strength screening study: iCIEF

| | iCIEF | | | |
|---|---|---|---|---|
| For- | GA (%) | | GB (%) | |
| mula- | | FT | | 40° C. |

| tion | | A | 5CY | 40° C. | | | A | FT 5CY | 2 | |
|---|---|---|---|---|---|---|---|---|---|---|
| No. | T0 | 3D | S | 2 W | 4 W | T0 | 3D | S | W | 4 W |
| Fb1 | 14.7 | 14.9 | 14.8 | 20.2 | 27.7 | 7.0 | 6.5 | 4.3 | 8.2 | 11.3 |
| Fb2 | 14.4 | 14.7 | 14.9 | 18.2 | 25.2 | 6.7 | 5.9 | 4.8 | 9.5 | 10.6 |
| Fb3 | 14.4 | 14.9 | 15.4 | 18.5 | 25.6 | 5.9 | 5.9 | 4.4 | 6.3 | 10.2 |
| Fb4 | 14.9 | 15.6 | 15.3 | 23.9 | 33.9 | 5.5 | 5.8 | 5.1 | 5.1 | 6.0 |
| Fb5 | 15.2 | 15.5 | 15.4 | 23.2 | 31.7 | 5.7 | 5.3 | 4.1 | 4.4 | 6.9 |
| Fb6 | 14.8 | 15.2 | 15.3 | 17.8 | 13.3 | 6.1 | 6.5 | 4.7 | 5.8 | 22.9 |
| Fb7 | 14.5 | 14.8 | 15.2 | 19.0 | 26.7 | 7.2 | 6.1 | 5.1 | 6.9 | 10.3 |
| Fb8 | 14.7 | 14.6 | 15.7 | 19.4 | 31.1 | 6.1 | 5.3 | 4.5 | 7.8 | 7.4 |
| Fb9 | 15.5 | 14.5 | 14.4 | 24.3 | 33.0 | 6.5 | 5.7 | 3.9 | 9.6 | 9.5 |
| Fb10 | 15.4 | 14.6 | 14.9 | 18.8 | 26.7 | 5.4 | 5.4 | 4.6 | 7.9 | 11.8 |
| Fb11 | 14.4 | 14.9 | 14.9 | 18.0 | 24.5 | 6.9 | 5.7 | 3.7 | 8.3 | 9.7 |

CE-SDS-NR&R: Table 18 provides the data from CE-SDS-NR&R testing for the excipients and surfactant strength screening study.

Compared with T0 samples, no obvious changes in non-reduced and reduced purity were observed in the 11 formulations with 3 days of agitation and 5 cycles of freeze/thaw stress.

After being incubated at 40° C. for 4 weeks, decreases of 6.0-10.7% in CE-SDS-NR purity were observed, amongst which, Fe6 (with sucrose) had the largest decrease (10.7%), while the Fe2, Fe3, Fe10 and Fe11 formulations performed better.

After being incubated at 40° C. for 4 weeks, decreases of 1.7-8.1% in CE-SDS-R purity were observed, amongst which Fe6 (with sucrose but no surfactant added) had the largest decrease (8.1%), while Fe2, Fe3, Fe10 and Fe11 formulations performed better.

TABLE 18
Excipients and surfactant strength screening study: CE-SDS-NR&R

| For- | CE-SDS-NR (Purity %) | | | | | CE-SDS-R (Purity %) | | | | |
|---|---|---|---|---|---|---|---|---|---|---|
| mula- | | | FT | | | | | FT | | |
| tion | | Agitation | 5 | 40° C. | 40° C. | | Agitation | 5 | 40° C. | T0 |
| No. | T0 | 3 D | CYS | 2 W | 4 W | T0 | 3 D | CYS | 2 W | 4 W |
| Fe1 | 93.6 | 93.9 | 93.5 | 88.6 | 86.0 | 98.4 | 98.2 | 98.4 | 97.0 | 96.4 |
| Fe2 | 93.7 | 93.9 | 93.6 | 90.9 | 86.5 | 98.4 | 98.3 | 98.4 | 97.3 | 96.4 |
| Fe3 | 93.7 | 93.5 | 93.5 | 91.0 | 86.1 | 98.4 | 98.3 | 98.3 | 97.3 | 96.4 |
| Fe4 | 93.7 | 93.9 | 93.6 | 90.3 | 85.2 | 98.4 | 98.2 | 98.4 | 97.4 | 95.9 |
| Fe5 | 93.6 | 93.9 | 93.3 | 90.1 | 86.0 | 98.5 | 98.3 | 98.3 | 97.3 | 96.3 |
| Fe6 | 93.3 | 94.2 | 92.7 | 90.9 | 82.6 | 98.5 | 98.3 | 98.4 | 97.4 | 90.4 |
| Fe7 | 93.8 | 94.1 | 93.6 | 90.5 | 85.9 | 98.5 | 98.2 | 98.4 | 97.2 | 95.8 |
| Fe8 | 93.7 | 93.0 | 92.5 | 89.1 | 85.2 | 98.4 | 97.9 | 98.3 | 96.7 | 95.2 |
| Fe9 | 93.5 | 93.4 | 93.4 | 88.8 | 84.2 | 98.3 | 98.2 | 98.3 | 96.5 | 95.8 |
| Fe10 | 93.3 | 93.7 | 93.5 | 90.7 | 86.2 | 98.4 | 98.3 | 98.4 | 97.3 | 96.1 |
| Fe11 | 93.6 | 93.7 | 93.6 | 92.2 | 87.6 | 98.4 | 98.2 | 98.4 | 97.3 | 96.7 |

Potency: Based on the above testing results, the Fe3 formulation (20 mM L-Histidine/L-Histidine monohydrochloride buffer, 4.500 (w/v) sorbitol, 0.02% (w/v) PS80, pH 6.5) was selected for potency testing. As shown in Table 19, obvious decrease in potency was found when IVX-01101 was stored at 40° C. for 4 weeks compared to T0.

TABLE 19
Excipients and surfactant strength screening study: potency

| | Binding_Antigen | |
|---|---|---|
| Formulation No. | T0 | 40° C.-4 W |
| Fe3 | 116% | 54% |

In the excipients and surfactant strength screening study, the appearance and sub-visible particles results showed that the IVX-01101 protein exhibited poor stability in the formulations with sucrose, sodium chloride, or L-Arginine hydrochloride. The appearance and sub-visible particles results showed that PS80 can prevent the generation of protein aggregates effectively. For formulations with sucrose, Fe1, Fe6 to Fe9 samples became slight yellow after 4 W incubation at 40° C.

The SE-UPLC results demonstrated that formulation with trehalose and sorbitol performed better than other formulations with 0.02% (w/v) PS80. In formulations with PS80, less than 0.02% (w/v) showed higher SEC monomer than samples without PS80. In addition, SEC monomer decreased with increased concentration of PS80 that were more than 0.04% (w/v). Fe2, Fe3 and Fe11 formulations showed less decrease in SEC monomer than other formulations.

The iCIEF results showed that formulations with sodium chloride or L-Arginine hydrochloride exhibited poor stability with Fe2, Fe3 and Fe11 showing less iCIEF main peak decrease than other formulations.

For CE-SDS-NR&R results, Fe2, Fe3 and Fe11 showed less CE-SDS purity decrease than other formulations.

In addition, results indicated that PS80 can prevent the generation of sub-visible or visible particles effectively, while higher concentrations (close to 0.06%) of PS80 also have some impact on SEC main peak. In order to balance the protection capability of PS80, a medium concentration (0.02%) was selected.

In summary, Fe2, Fe3 and Fe11 formulations performed the best and results were comparable. Even though Fe3 (trehalose) is a suitable excipient, it was not recommended due to relatively high material cost compared to sucrose or sorbitol. Finally, 20 mM L-Histidine/L-Histidine monohydrochloride buffer, 4.5% (w/v) sorbitol, 0.02% (w/v) PS80, 0.05 mM disodium EDTA at pH 6.5 was chosen as the optimal formulation for IVX-01101 formulation.

Formulation Confirmation Study: A study was performed using Day 14 and Day 16 production bioreactor material that was purified using anion exchange chromatography (AEX) under conditions with and without additional salt. The material generated followed the same process as what would be used for the GMP manufacturing of the pivotal study and commercial material except for the D14 vs D16 production bioreactor material and minor variation in AEX salt conditions. The study was performed at 60 mg/mL concentration as the higher concentration is considered a worst case for the product quality and stability. The details of the study are provided below:
Aim: To use the final process material for confirming the formulation.
Method: to evaluate protein stability under thermal stress conditions below.
Target filling volume: 1.15 mL/2 R vial (Label volume: 1 mL).
Study Period: 1 Month.

TABLE 20
Short term formulation confirmation study

| | | | | Sampling Points and Assay | | | |
|---|---|---|---|---|---|---|---|
| Study | Formulation | Concentration | Conditions | T0 | 1 W | 2 W | 4 W |
| D14 | F01: | 60 mg/mL | 40 C. | X, (Y) | X, (Y) | X, (Y) | X, (Y) |
| stability | 20 mM Histidine | | 25 C. | | / | / | X, (Y) |
| | buffer, 5% (w/v) | | 5 C. | | | | (X, Y) |
| | sorbitol, 0.02% (w/v) | | (control) | | | | |
| | PS80, pH 6.5 | | | | | | |
| D16 | F01: 20 mM | 60 mg/mL | 40 C. | X, (Y) | X, (Y) | X, (Y) | X, (Y) |
| stability | Histidine buffer, 5% | | 25 C. | | / | / | X, (Y) |
| | (w/v) sorbitol, 0.02% | | 5 C. | | | | (X, Y) |
| | (w/v) PS80, pH 6.5 | | (control) | | | | |
| | F02: 20 mM | | | | | | |
| | Histidine buffer, 5% | | | | | | |
| | (w/v) sorbitol, 0.02% | | | | | | |
| | (w/v) PS80, 0.05mM | | | | | | |
| | EDTA, pH 6.5 | | | | | | |
| | F03: 20 mM | | | | | | |
| | Acetate, 5 % (w/v) | | | | | | |
| | sorbitol, 0.02% (w/v) | | | | | | |
| | PS80, pH 5.0 | | | | | | |

X = Appearance, SE-UPLC, CE-SDS (NR & R);
Y = Potency, PS80 concentration
(X, Y) : samples reserved, testing if needed.

The results from the different analytical methods are shown below:

TABLE 21
IVX-01101 Stability in Different Formulations Monitored by SE-UPLC method

| | | | | SEC-UPLC main peak/HMW/LMW % | | | | | |
|---|---|---|---|---|---|---|---|---|---|
| Sample | Formulation buffer | | Process | T0 | 40 C.-1 W | 40 C.-2 W | 40 C.-4 W | 25 C.-4 W | 5 C.-4 W |
| F1 | 60 | 20 mM | D14 + | 99.6/ | 97.1(↓2.5)/ | 96.2(↓3.4)/ | 95.5(↓4.1)/ | 98.2(↓1.4)/ | 99.0(↓0.6)/ |
| | mg/ | Histidine | NaCl | 0.4/ | 2.3(↑1.9)/ | 3.0(↑2.6)/ | 3.3(↑2.9)/ | 1.4(↑1.0)/ | 0.6(↑0.2)/ |
| | mL | buffer, 5% | | ND | 0.6(↑0.6) | 0.8(↑0.8) | 1.2(↑1.2) | 0.4(↑0.4) | 0.4(↑0.4) |
| F2 | | (w/v) sorbitol, | D14 | 99.4/ | 96.8(↓2.6)/ | 96.0(↓3.4)/ | 95.2(↓4.2)/ | 97.9(↓1.5)/ | 98.8(↓0.6)/ |
| | | 0.02% (w/v) | | 0.6/ | 2.7(↑1.9)/ | 3.2(↑2.6)/ | 3.6(↑3.0)/ | 1.7(↑1.1)/ | 0.9(↑0.3)/ |
| | | PS80, pH 6.5, | | ND | 0.6(↑0.6) | 0.8(↑0.8) | 1.2(↑1.2) | 0.4(↑0.4) | 0.3(↑0.3) |
| F3 | | 20 mM | D16 + | 99.4/ | 96.3(↓3.1)/ | 95.7(↓3.7)/ | 94.8(↓4.6)/ | 97.6(↓1.8)/ | 98.6(↓0.8)/ |
| | | Histidine | NaCl | 0.6/ | 3.0(↑2.4)/ | 3.5(↑2.9)/ | 4.0(↑3.4)/ | 2.0(↑1.4)/ | 0.9(↑0.3)/ |
| | | buffer, 5% | | ND | 0.6(↑0.6) | 0.8(↑0.8) | 1.2(↑1.2) | 0.5(↑0.5) | 0.4(↑0.4) |
| | | (w/v) sorbitol, | | | | | | | |
| | | 0.02% (w/v) | | | | | | | |
| | | PS80, pH 6.5 | | | | | | | |
| F4 | | 20 mM | D16 + | 99.4/ | 97.7(↓1.7)/ | 97.2(↓2.2)/ | 96.1(↓3.3)/ | 98.3(↓1.1)/ | 98.7(↓0.7)/ |
| | | Histidine | NaCl | 0.6/ | 1.8(↑1.2)/ | 2.1(↑1.5)/ | 2.9(↑2.3)/ | 1.3(↑0.7)/ | 0.9(↑0.3)/ |
| | | buffer, 5% | | ND | 0.5(↑0.5) | 0.7(↑0.7) | 1.0(↑1.0) | 0.4(↑0.4) | 0.4(↑0.4) |
| | | (w/v) sorbitol, | | | | | | | |
| | | 0.02% (w/v) | | | | | | | |
| | | PS80, 0.05mM | | | | | | | |
| | | EDTA, pH 6.5 | | | | | | | |
| | | (Lead) | | | | | | | |
| F5 | | 20 mM | D16 + | 99.2/ | 96.2(↓3.0)/ | 95.1(↓4.1)/ | 93.6(↓5.6)/ | 97.2(↓2.0)/ | 98.3(↓0.9)/ |
| | | Acetate, 5 % | NaCl | 0.8/ | 3.1(↑2.3)/ | 4.0(↑3.2)/ | 4.8(↑4.0)/ | 2.3(↑1.5)/ | 1.4(↑0.6)/ |
| | | (w/v) sorbitol, | | ND | 0.6(↑0.6) | 0.9(↑0.9) | 1.6(↑1.6) | 0.4(↑0.4) | 0.4(↑0.4) |
| | | 0.02% (w/v) | | | | | | | |
| | | PS80, pH 5.0 | | | | | | | |

When results are compared amongst the F3, F4, and F5 formulations, the formulation containing EDTA helped to slightly decrease aggregate levels. 20 mM Acetate performed slightly worse when compared to 20 mM histidine after incubation at 40 C/25 C/5 C for 4 W; however, product quality was still acceptable.

TABLE 22
IVX-01101 Stability in Different Formulations Monitored by Reduced and Non-Reduced CE-SDS method

| | | | | CE-SDS-NR purity % | | | | | | CE-SDS-R purity % | | | | | |
|---|---|---|---|---|---|---|---|---|---|---|---|---|---|---|---|
| | | | | | 40 C.- | 40 C.- | 40 C.- | 25 C.- | 5 C.- | | 40 C.- | 40 C.- | 40 C.- | 25 C.- | 5 C.- |
| Sample | Formulation buffer | | Process | T0 | 1 W | 2 W | 4 W | 4 W | 4 W | T0 | 1 W | 2 W | 4 W | 4 W | 4 W |
| F1 | 60 | 20 mM | D14 + | 97.3 | 95.0 | 93.1 | 90.0 | 95.4 | 96.5 | 95.4 | 95.1 | 93.8 | 93.1 | 94.9 | 95.0 |
| | mg/ | Histidine | NaCl | | (↓2.3) | (↓4.2) | (↓7.3) | (↓1.9) | (↓0.8) | | | (↓1.6) | (↓2.3) | | |
| | mL | buffer, 5% | | | | | | | | | | | | | |
| F2 | | (w/v) sorbitol, | D14 | 96.6 | 94.3 | 92.7 | 89.1 | 94.6 | 95.9 | 95.1 | 94.8 | 93.4 | 92.4 | 94.2 | 94.9 |
| | | 0.02% (w/v) | | | (↓2.3) | (↓3.9) | (↓7.5) | (↓2.0) | (↓0.7) | | | (↓1.7) | (↓2.7) | | |
| | | PS80, pH 6.5, | | | | | | | | | | | | | |
| F3 | | 20 mM | D16 + | 96.2 | 93.7 | 92.2 | 88.5 | 93.8 | 95.4 | 94.9 | 94.0 | 92.9 | 91.5 | 93.2 | 93.9 |
| | | Histidine | NaCl | | (↓2.5) | (↓4.0) | (↓7.7) | (↓2.4) | (↓0.8) | | | (↓2.0) | (↓3.4) | | |
| | | buffer, 5% | | | | | | | | | | | | | |
| | | (w/v) sorbitol, | | | | | | | | | | | | | |
| | | 0.02% (w/v) | | | | | | | | | | | | | |
| | | PS80, pH 6.5 | | | | | | | | | | | | | |
| F4 | | 20 mM | D16 + | 96.2 | 94.4 | 93.1 | 90.3 | 94.9 | 95.5 | 94.7 | 94.5 | 92.9 | 92.6 | 93.9 | 94.3 |
| | | Histidine | NaCl | | (↓1.8) | (↓3.1) | (↓5.9) | (↓1.3) | (↓0.7) | | | (↓1.8) | (↓2.1) | | |
| | | buffer, 5% | | | | | | | | | | | | | |
| | | (w/v) sorbitol, | | | | | | | | | | | | | |
| | | 0.02% (w/v) | | | | | | | | | | | | | |
| | | PS80, 0.05mM | | | | | | | | | | | | | |
| | | EDTA, pH 6.5 | | | | | | | | | | | | | |
| | | (Lead) | | | | | | | | | | | | | |
| F5 | | 20 mM | D16 + | 96.2 | 94.0 | 91.8 | 88.2 | 94.3 | 95.3 | 94.6 | 94.1 | 92.4 | 91.1 | 93.6 | 94.1 |
| | | Acetate, 5 % | NaCl | | (↓2.2) | (↓4.4) | (↓8.0) | (↓1.9) | (↓0.9) | | | (↓2.2) | (↓3.5) | | |
| | | (w/v) sorbitol, | | | | | | | | | | | | | |
| | | 0.02% (w/v) | | | | | | | | | | | | | |
| | | PS80, pH 5.0 | | | | | | | | | | | | | |

When compared with formulations F3, F4, and F5, the formulation with EDTA helped to decrease purity loss.

TABLE 23
IVX-01101 Stability in Different Formulations Monitored by Polysorbate concentration method and Potency by ELISA method

| | | | | PS80 Concentration % | | | | Potency by ELISA % | | | |
|---|---|---|---|---|---|---|---|---|---|---|---|
| | | | | | 40 C.- | 25 C.- | 5 C.- | | 40 C.- | 25 C.- | 5 C.- |
| Sample | Formulation buffer | | Process | T0 | 4 W | 4 W | 4 W | T0 | 4 W | 4 W | 4 W |
| F1 | 60 | 20 mM Histidine buffer, 5% (w/v) | D14 + | NA | | | | | | | |
| | mg/mL | sorbitol, 0.02% (w/v) PS80, pH 6.5 | NaCl | | | | | | | | |
| F2 | | | D14 | | | | | | | | |
| F3 | | 20 mM Histidine buffer, 5% (w/v) | D16 + | 0.018 | <LOQ | <LOQ | 0.017 | 105 | 43 | 86 | 113 |
| | | sorbitol, 0.02% (w/v) PS80, pH 6.5 | NaCl | | | | | | | | |
| F4 | | 20 mM Histidine buffer, 5% (w/v) | D16 + | 0.020 | 0.023 | 0.022 | 0.020 | 103 | 49 | 73 | 106 |
| | | sorbitol, 0.02% (w/v) PS80, | NaCl | | | | | | | | |
| | | 0.05 mM EDTA, pH 6.5 | | | | | | | | | |
| F5 | | 20 mM Acetate, 5% (w/v) sorbitol, | D16 + | NA | | | | | | | |
| | | 0.02% (w/v) PS80, pH 5.0 | NaCl | | | | | | | | |

The formulation development of IVX-01101 consisted of multiple steps: a pH/buffer screening study and an excipients and surfactant strength evaluation study, followed by adjustments in concentrations during formulation confirmation studies. Multiple physicochemical and biochemical methods were involved for choosing a lead formulation, including appearance, pH, protein concentration, osmolality, DSC, SE-UPLC, sub-visible particles (MFI), iCIEF, CE-SDS (NR&R) and potency.

According to the pH/buffer screening results, 20 mM L-Histidine/L-Histidine monohydrochloride buffer at pH 6.5 was selected as the lead pH/buffer system. According to the excipients screening and surfactant strength evaluation study, IVX-01101 protein in 20 mM L-Histidine/L-Histidine monohydrochloride buffer with sorbitol was more stable than other formulations. Based on the formulation confirmation study, the stability profiles of the lead formulations look good. Overall, 5 to 60 mg/mL IVX-01101 protein in 20 mM L-Histidine/L-Histidine monohydrochloride buffer, 4.5% (w/v) sorbitol, 0.02% (w/v) PS80, 0.05 mM disodium EDTA at pH 6.5±0.4 is considered as a lead formulation for the canine antibody, IVX-01101.

### Example 3: IVX-01286 Multi-Dose Formulation Feasibility Study

### Materials and Methods

The purpose of the feasibility study is to evaluate the impact of preservatives/antimicrobial types and strengths in different formulations on IVX-01286 antibody product quality and stability to enable multi-dose formulation. The study was performed to identify formulations with preservatives that demonstrate prolonged stability comparable to ones without preservative and evaluates preservative concentrations that have been shown to protect the formulation against microbial growth. Preservatives at effective anti-microbial concentrations have been shown to cause protein aggregation and degradation for certain classes of proteins, and therefore multiple analytical techniques were used for this study to monitor antibody instability under those anti-microbial concentrations.

Stress studies including incubation at temperature conditions of 40° C. and 25° C. for a short term, and control recommended storage condition at 2˜8° C. over 6 months were conducted to characterize the molecules by appearance, pH, protein concentration (conc.), osmolality, sub-visible particles by high-accuracy liquid particle counting (HIAC), size exclusion-high performance liquid chromatography (SE-HPLC), imaged capillary isoelectric focusing (iCIEF), caliper-sodium dodecyl sulfate (non-reduced & reduced) (caliper-SDS-NR & R) analysis and potency by enzyme-linked immunosorbent assay (ELISA).

The IVX-01286 drug substance (DS) information used in the formulation development study is listed in Table 24.

TABLE 24
IVX-01286 molecule information

| Material | Manufactured | | | |
|---|---|---|---|---|
| name | by | Lot No. | Batch size | Formulation |
| IVX- | WuXi | 7671- | 10 L WuXian | 27.77 mg/mL, |
| 01286 | Biologics PS | 1W230323 | Express | 20 mM |
| | | | (transient | acetate buffer, 8% |
| | | | expression | (w/v) sucrose, |
| | | | system) | pH 5.0 |

Appearance: The appearance of all samples, including color, clarity, and visible particles, was examined against black and white backgrounds using YB-2 Clarity Detector. Light intensity of light box was set as 2000˜3750 lux.

Protein Concentration: Protein concentration (conc.) was measured by Lunatic® UV absorption instrument (Unchained Labs). According to the Lambert-Beer law, the concentration of a protein solution can be calculated based on its absorbance at a given wavelength, the cuvette cell path length, and extinction coefficient value. The absorbance in 280 nm relies on the absorption properties of the aromatic amino acid residues in protein. After loading a 2.5 μL sample volume, Lunatic measured the absorbance in two cuvettes in parallel and calculated the concentration value. The extinction coefficient used in this evaluation was 1.52 mL*mg⁻¹*cm⁻¹.

pH: Sample pH was measured using a pH meter with a glass electrode at 25° C. The pH meter was calibrated prior to use with three different standard buffers (pH 4.01, 7.00 and 9.21). The slope of calibration was between 95.0% to 105.0%, and the zero drift was between −60.0 mV to +60.0 mV. Each sample was tested twice, and an average result was reported.

Osmolality: Osmolality was measured using Osmotech Pro. The sample was directly test without dilution. Before and after the tests, the testing accuracy of the osmometer was confirmed with a Clinitrol 290 mOsm/kg reference solution. The sample volume for testing was 30 μL and only one test was performed for each sample.

HIAC: A Liquid Particle Counting Systems (HIAC 9703+) was utilized to measure the size and counts of sub-visible particles in a bio-safety cabinet. To avoid introducing air bubbles and interference during examination, all samples were held in the bio-safety cabinet for at least 0.5 hour before testing. Each sample was tested for four consecutive runs, 0.4 mL each/run. Per the pharmacopeial method, the data from the first run was discarded, and the results were reported as an average number of the remaining 3 runs, for particles of ≥2 μm, ≥10 μm and ≥25 μm per mL (method conforms to United States Pharmacopoeia (USP) <788> Particulate matter in injections.

SE-HPLC: Size exclusion high performance liquid chromatography (SE-HPLC) is a purity analysis method that separates proteins based on their sizes. Measurements were performed on an Agilent HPLC system with a SEC column (300×7.8 mm, 5 μm). The sampler temperature was set to 5±3° C. and the column oven temperature was set as 25±3° C. The mobile phase was 50 mM PB, 300 mM NaCl, pH 6.8±0.1 and the flow rate was set as 1.0 mL/min. Samples were diluted to 10 mg/mL with mobile phase and 100 μg samples were injected. Detection wavelength was set at 280 nm and the run time was 20 minutes. Data was analyzed by Agilent CDS Software.

iCIEF: Imaged capillary isoelectric focusing (iCIEF) is a purity analysis method used to monitor the percentage of charge variant species or charge heterogeneity of the antibody. The isoelectric point (pI) is an intrinsic property of a specific protein and is the pH at which the protein molecule does not carry net electrical charge. Under an external electric field, the charge variants move along a continuous pH gradient formed by ampholytes and stop at where the pH equals the protein's pI.

20 μg sample is mixed with 80 μL master mix, which is composed of the carrier ampholyte, pI markers, methyl cellulose and urea. The mixture is then analyzed with the iCE3 from ProteinSimple with a fluorocarbon (FC)-coated whole-column detection capillary. Detection wavelength was set at 280 nm to evaluate the charge variants distribution in different pI range. The raw data was processed with Chrom Perfect Analysis and the relative percentage of the charge variants was reported.

Caliper-SDS-NR: Caliper Sodium Dodecyl Sulfate (Caliper-SDS-NR) was performed based on a PerkinElmer Caliper automated electrophoresis that allows protein separation by size. In the non-reduced microchip CE-SDS analysis, samples were diluted to 1 mg/mL with pure water. 2 μL of diluted samples were mixed with 7 μL of the sample denaturing solution (the mixture of sample buffer, 10% SDS solution and 100 mM N-Ethylmaleimide (NEM) with the volume ratio of 100:10:4). The well-mixed samples were incubated at 70° C. for 10 minutes, then spin at 14000 rpm for 1 minute at room temperature. 35 μL of water was added to each sample, vortexed for 10 seconds and centrifuged at 14000 rpm for 1 minute. 42 μL of the prepared samples were transferred to a 96-well plate, centrifuged at 4000 rpm for 15 minutes, placed onto a Labchip GXII's plate holder, separated and detected in the HT Protein Express LabChip filled with destain-gel, gel-dye and lower marker. Results was analyzed with LabChip GX Reviewer software.

Caliper-SDS-R: Caliper Sodium Dodecyl Sulfate (Caliper-SDS-R) was performed based on a PerkinElmer Caliper automated electrophoresis that allows protein separation by size after reduction of sample. In the reduced microchip CE-SDS analysis, samples were diluted to 1 mg/mL with pure water. 2 μL of diluted samples were mixed with 7 μL of the sample denaturing solution (the mixture of sample buffer, 10% SDS solution and 1 M dithiothreitol as to reduction agent with the volume ratio of 100:10:4). The well-mixed samples were incubated at 70° C. for 10 minutes, then spin at 14000 rpm for 1 minute at room temperature. 35 μL of water was added to each sample, vortexed for 10 seconds and centrifuged at 14000 rpm for 1 minute. 42 μL of the prepared samples were transferred to a 96-well plate, centrifuged at 4000 rpm for 15 minutes, placed onto a Labchip GXII's plate holder, separated and detected in the HT Protein Express LabChip filled with destain-gel, gel-dye and lower marker. Results was analyzed with LabChip GX Reviewer software.

Potency: This assay is an ELISA method for the binding to canine NGF (nerve growth factor) potency analysis of IVX-01286 monoclonal antibody. Samples, assay control (AC) and standard (STD) at appropriate dilutions are loaded onto to the antigen canine NGF protein coated 96-well high binding plate. After washing plate, Peroxidase-AffiniPure Rabbit Anti-Dog IgG (H+L) antibody added to the wells allowing interaction with IVX-01286 captured during the previous step. After a final washing step, the TMB substrate solution is loaded into wells. TMB specifically reacts with peroxide in the presence of peroxidase and produces a colorimetric signal that is proportional to the amount of IVX-01286 bound to the wells.

Dose response curves of samples, AC and STD are plotted according to a 4-parameter logistic (Auto-Estimate) regression model using the SoftMax Pro software. Individual EC50 of samples, AC and STD is obtained respectively on the same plate. Relative binding activity of the sample and AC is calculated by the following formula:

$$Relative binding activity of the samples / AC \% = \frac{{EC}_{50} of the STD}{{EC}_{50} of the samp1es / AC} \times 100 \%$$

Take out the Canine NGF protein stock solution from ≤−60° C. refrigerator, equilibrate to room temperature, avoid repeated freezing and thawing. To prepare the antigen working solution, dilute the antigen to 3 μg/mL in coating buffer. Coat the 96-well plates with 3 μg/mL Canine NGF protein solution at 100 μL/well. Seal plates with plate seal film and incubate the plate for 16-86 h at 2-8° C. Remove 96-well assay plate from 2-8° C. Wash plate 6 times with approximately 300 μL/well of 0.05% PBST using multi-channel pipette or plate washer. If using multi-channel pipette, remove residual by lightly blotting the plate onto paper towel. If using a plate washer, wash 3 times with the plate being placed forward and then turn the plate around and wash another 3 times with the plate being placed reverse. Remove residual by lightly blotting the plate onto paper towel. When using a plate washer. Add 200 μL/well of blocking buffer (1% BSA/PBS) to the plate. Seal the plate with plate sealer, shake plate with plate shaker at 220 RPM, 25° C. for 1-3 h. Aliquot each dilution points to set up duplicated wells. Transfer 100 μL of diluted STD, AC and Samples into separate wells in the 96-well plate. Seal the plate. Incubate plate for 70±20 min on plate shaker at approximately 220 RPM at 25° C. Add 100 μL/well of Peroxidase-AffiniPure Rabbit Anti-Dog IgG (H+L) antibody work solution (1:50 k) to the plate. Seal the plate with plate sealer. Incubate plate for 70±20 min on plate shaker at approximately 220 RPM at 25° C. Remove 96-well assay plate and wash plate. Add 100 μL/well of TMB to all wells. Stop reaction after about 10-20 min. Then add 100 μL/well of 1 M HCl to stop the reaction. Place plate into plate reader within 45 min (≤45 min) after stop reaction (Read absorbance at 450 nm).

### Study Design

In this feasibility study report, thermal stress study was examined to characterize the IVX-01286 molecule. A brief description of the study is shown in Table 25.

TABLE 25
The study design of IVX-01286 formulation feasibility study

| Study | Description |
|---|---|
| Formulation | Different types and strengths of excipients ( Stabilizers: |
| feasibility | sorbitol, sucrose; Preservatives: benzyl alcohol, and m- |
| study | cresol) and two different protein concentrations in the |
| | pH/buffer systems were investigated under stress |
| | conditions (40° C. for up to 4 weeks, 25° C. for up to 3 |
| | months, and 5° C. for up to 6 months). Samples were |
| | tested for appearance, protein concentration, pH, |
| | osmolality at T0 only, HIAC, SE-HPLC, iCIEF, caliper- |
| | SDS-NR & R, and potency. |

### Formulation Screening Study

Objective: The formulation feasibility study was aimed at evaluating stability of IVX-01286 molecule, and selecting the optimal pH/buffer and excipient system with 0.02% (w/v) PS80 and 0.05 mM EDTA with preservative to identify preservative formulation with good stability profile.

Sample Preparation: IVX-01286 DS (Lot No.: 7671-1 W230323) was used for this study. It was buffer-exchanged into the prepared 20 mM histidine buffer (pH 6.6) and 20 mM acetate buffer (pH 5.0) first. Stock solutions of 5% (w/w) PS80, 10 mM EDTA, 20% (w/w) sorbitol and 40% (w/w) sucrose were prepared. The required amounts of DS, excipient stock solutions and antimicrobials were calculated, weighed and well-mixed based on the formulation recipes listed in Table 26. Each of the formulation solutions were filtered with a 0.22 μm PES filter and filled into 2 mL glass vials (1 mL per vial), then stoppered, capped and labeled immediately. All sample filtration and aliquoting was carried out with aseptic procedures in a bio-safety cabinet.

TABLE 26
Formulation candidates list of formulation feasibility study

| | Protein | | | | | | |
|---|---|---|---|---|---|---|---|
| | concentration | Buffer | Target | | | | |
| No. | (mg/mL) | system | pH | Excipient | Surfactant | Chelator | Antimicrobial |
| F01 | 60 | 20 mM | 6.6 | 5% (w/v) | 0.02% | 0.05 mM | NA |
| F02 | 60 | Histidine | | sorbitol | (w/v) | EDTA | 0.9% (w/v) |
| | | | | | PS80 | | benzyl alcohol |
| F03 | 60 | | | | | | 1.2% (w/v) |
| | | | | | | | benzyl alcohol |
| F04 | 60 | | | | | | 0.4% (w/v) |
| | | | | | | | m-cresol |
| F05 | 30 | | | 9% (w/v) | | | 1.2% (w/v) |
| | | | | sucrose | | | benzyl alcohol |
| F06 | 60 | 20 mM | 5.0 | | | | 1.2% (w/v) |
| | | Acetate | | | | | benzyl alcohol |

Study Parameters: The sampling and testing plan for the formulation feasibility study are shown in Table 27. For thermal stress, samples were stored at 40° C. for up to 4 weeks, 25° C. for up to 3 months and 5° C. for up to 6 months. Testing items including appearance, protein concentration, pH, osmolality, HIAC, SE-HPLC, iCIEF, caliper-SDS-NR & R and potency were performed in this study.

TABLE 27
Sampling and testing plan of formulation feasibility study

| Attribute | Condition | T0 | Sampling Points and Assays | |
|---|---|---|---|---|
| Thermal | 40° C. | X, Y, | 2 weeks | 4 weeks |
| | | (Z) | X, Y | X, Y, (Z) |

| | | | | |
|---|---|---|---|---|
| | 25° C. | 1 month | 2 months | 3 months |
| | | X | X, Y | X, Y, (Z) |
| Control | 2~8° C. | 1 month | 3 months | 6 months |
| | | X | X, Y | X, Y, (Z) |

X = Appearance, pH, protein concentration, SE-HPLC, Caliper (NR & R), iCIEF, Osmolality at T = 0 only
Y = Sub-visible particles (HIAC)
Z = ELISA activity (only for the selected formulations)

### Results

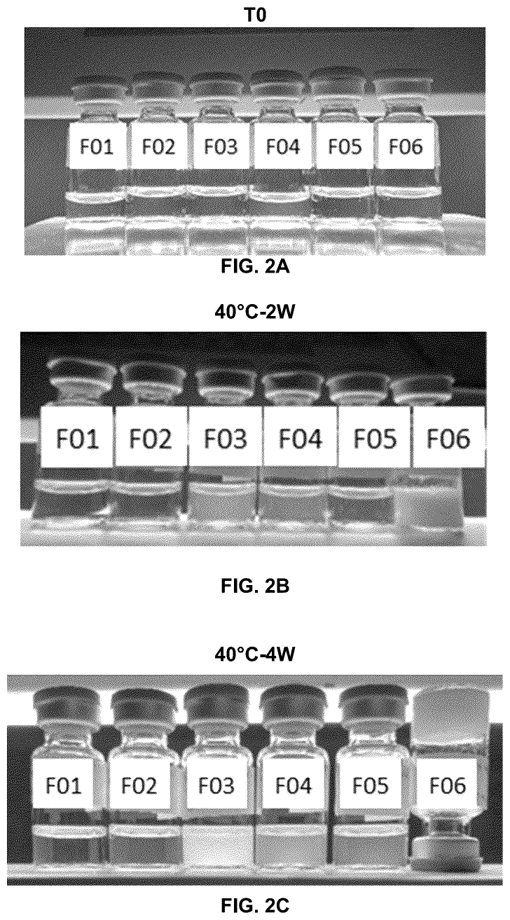

Appearance: The data summary of the appearance testing for the formulation screening study is provided in Table 28 and FIGS. 2A-2C. All formulations remained colorless, slightly opalescent and free of visible particles at T0. After incubation at 40° C. for 4 weeks, F01/F02/F05 samples remained colorless, slightly opalescent and free of visible particles, while the opalescence of F03 and F04 was opalescent and obviously heavier than T0. Notably, F06 sample became gel like, no further test could be conducted.

The samples subjected post incubation at 25° C. for 3 months and 5° C. for 6 months, maintained a colorless, slightly opalescent and free of visible particles appearance.

TABLE 28
Formulation feasibility study results: appearance

| Form- | Appearance | | | | | | | | |
|---|---|---|---|---|---|---|---|---|---|
| ulation | | 40° C. | | 25° C. | | | 5° C. | | |
| No. | T0 | 2 W | 4 W | 1 M | 2 M | 3 M | 1 M | 3 M | 6 M |
| F01 | C, SOL, | C, SOL, | C, SOL, | C, SOL, | C, SOL, | C, SOL, | C, SOL, | C, SOL, | C, SOL, |
| | FP | FP | FP | FP | FP | FP | FP | FP | FP |
| F02 | C, SOL, | C, SOL, | C, SOL, | C, SOL, | C, SOL, | C, SOL, | C, SOL, | C, SOL, | C, SOL, |
| | FP | FP | FP | FP | FP | FP | FP | FP | FP |
| F03 | C, SOL, | C, OL, | C, OL, | C, SOL, | C, SOL, | C, SOL, | C, SOL, | C, SOL, | C, SOL, |
| | FP | FP | FP | FP | FP | FP | FP | FP | FP |
| F04 | C, SOL, | C, SOL, | C, OL, | C, SOL, | C, SOL, | C, SOL, | C, SOL, | C, SOL, | C, SOL, |
| | FP | FP | FP | FP | FP | FP | FP | FP | FP |
| F05 | C, SOL, | C, SOL, | C, SOL, | C, SOL, | C, SOL, | C, SOL, | C, SOL, | C, SOL, | C, SOL, |
| | FP | FP | FP | FP | FP | FP | FP | FP | FP |
| F06 | C, SOL, | W, OG | W, OG | C, SOL, | C, SOL, | C, SOL, | C, SOL, | C, SOL, | C, SOL, |
| | FP | | | FP | FP | FP | FP | FP | FP |

Abbreviation: C = Colorless; SOL = Slightly opalescent liquid; W = White; OG = Opalescent gel; OL = Opalescent liquid; FP = Free of visible particles; EFP = Essentially free of visible particles (≤3); NEFP = Not essentially free of visible particles (>3); M = Month; W = Weeks.

Protein concentration: The protein concentration of the formulations (F01-F06) was assessed over several months at different thermal conditions as shown in Table 29 below. The protein concentration (approximately 60 mg/mL and 30 mg/mL) of all candidates remained relatively consistent after the incubation at 40° C., 25° C. and 5° C. compared to those at T0, indicating that protein concentration was stable under the three thermal stress conditions.

TABLE 29
Formulation feasibility study results: protein concentration

| Form- | Protein concentration (mg/ml) | | | | | | | | |
|---|---|---|---|---|---|---|---|---|---|
| ulation | | 40° C. | | 25° C. | | | 5° C. | | |
| No. | T0 | 2 W | 4 W | 1 M | 2 M | 3 M | 1 M | 3 M | 6 M |
| F01 | 60.6 | 62.7 | 63.6 | 61.3 | 60.7 | 60.9 | 61.7 | 61.5 | 61.2 |
| F02 | 60.5 | 61.1 | 61.7 | 60.9 | 60.5 | 60.5 | 60.7 | 60.5 | 60.7 |
| F03 | 60.5 | 61.4 | 63.9 | 60.9 | 60.6 | 60.7 | 61.0 | 60.6 | 61.0 |
| F04^{[1]} | 61.8 | 63.3 | 64.2 | 62.5 | 61.9 | 61.5 | 61.7 | 61.6 | 60.8 |
| F05 | 30.5 | 31.3 | 32.0 | 30.9 | 30.8 | 30.6 | 30.9 | 30.6 | 30.8 |
| F06 | 60.5 | NT | NT | 60.9 | 60.7 | 60.6 | 61.0 | 60.6 | 60.4 |

Abbreviation: W = Week; M = Month; NT = Not test.
Note:
^{[1]}The addition of m-cresol affected the determination of F04 prescription concentration, all the concentration of F04 were deducted the m-cresol absorption, and the concentration was within the reasonable range.

pH and osmolality: As shown in Table 30, the pH of the tested formulations remained relatively stable during the study, except F06, which was not tested under 40° C. condition. The osmolality (non-stability indicative parameter) of all samples was 363-536 mOsm/kg. An increase in osmolality with increasing concentration of antimicrobial preservative and drug substance was observed. These are considered slightly hypertonic and not expected to cause any patient issues during parenteral administration.

TABLE 30
Formulation feasibility study results: pH and osmolality.

| | pH | | | | | | | | | Osmolality |
|---|---|---|---|---|---|---|---|---|---|---|
| | | 40° C. | | 25° C. | | | 5° C. | | | (mOsm/kg) |
| No. | T0 | 2 W | 4 W | 1 M | 2 M | 3 M | 1 M | 3 M | 6 M | T0 |
| F01 | 6.4 | 6.6 | 6.6 | 6.5 | 6.6 | 6.5 | 6.5 | 6.5 | 6.6 | 363 |
| F02 | 6.5 | 6.6 | 6.5 | 6.5 | 6.6 | 6.5 | 6.5 | 6.5 | 6.5 | 460 |
| F03 | 6.5 | 6.6 | 6.5 | 6.5 | 6.6 | 6.5 | 6.5 | 6.5 | 6.5 | 491 |
| F04 | 6.4 | 6.6 | 6.5 | 6.5 | 6.5 | 6.5 | 6.5 | 6.5 | 6.5 | 399 |
| F05 | 6.5 | 6.6 | 6.5 | 6.5 | 6.5 | 6.6 | 6.5 | 6.5 | 6.5 | 483 |
| F06 | 5.0 | NT | NT | 5.1 | 5.1 | 5.2 | 5.1 | 5.1 | 5.1 | 536 |

Abbreviation: W = Week; M = Month; NT = Not test.

SE-UPLC: The data summary of SE-HPLC testing results for the formulation feasibility study is shown in Table 31. All candidates showed a decline in the % monomer (up to 12.5% for adding m-cresol as antimicrobial) after 4 weeks of incubation at 40° C. This change was the most pronounced with the addition of antimicrobial agents while F01 without any antimicrobial agents showed the smallest changes in monomer purity. F04 (12.5%↓) and F05 (7.1%↓) showed more decline in % monomer, F06 at 40° C. became a gel-like substance. When stored at 25° C. for 3 months, the % monomer in all formulation had slightly decline compared to T0, and F06 has the lowest % monomer of 97.0%.

Together, the SE-HPLC analysis indicated that IVX-01286 protein tended to form polymers of high molecular weight under thermal stress conditions, especially at 40° C., and least stable under the interaction of acetate buffer, 9% (w/v) sucrose and to some extent at slightly higher preservative level (i.e. 1.2% benzyl alcohol) comparted to control. Besides, 0.4% (w/v) m-cresol had more impact than 1.2% (w/v) benzyl alcohol on the SEC % monomer when at the same protein concentration based on the 40° C. 4 W data.

All formulations were stable at the control recommended storage condition of 5° C. for up to 6 months based on % monomer.

iCIEF. The data summary of iCIEF testing results for the formulation feasibility study is shown in Table 32. The measured pI of all samples was 5.4. A significant decline in the main peak % (up to 29.2% for m-cresol and 27.2% for benzyl alcohol) was seen in all formulation systems after storage at 40° C. for 4 weeks. The formulation with 0.9% benzyl alcohol performed comparable to the corresponding formulation without benzyl alcohol within the expected variability of the analytical method. After incubation at 25° C. and 5° C. thermal conditions, all formulations can be observed a decline in the main peak %, among them F04 showed the highest decline of 22.9% at 25° C.-3M and 8.4% at 5° C.-6M. However, all formulations showed similar decline and were considered relatively stable at the control recommended storage condition of 5° C. for up to 6 months.

TABLE 31
Formulation feasibility study results: SE-HPLC

| | SE-HPLC (Monomer %/HMW %/LMW %) | | | | | | | | |
|---|---|---|---|---|---|---|---|---|---|
| | | 40° C. | | 25° C. | | | 5° C. | | |
| No. | T0 | 2 W | 4 W | 1 M | 2 M | 3 M | 1 M | 3 M | 6 M |
| F01 | 99.6/ | 98.8( ↓ 0.8)/ | 97.6( ↓ 2.0)/ | 99.2/ | 99.1/ | 98.9( ↓ 0.7)/ | 99.5/ | 99.5/ | 99.3/ |
| | 0.3/0.1 | 1.1/0.1 | 1.6/0.8 | 0.6/0.2 | 0.7/0.2 | 0.8/0.3 | 0.5/0.0 | 0.5/0.0 | 0.6/0.1 |
| F02 | 99.4/ | 98.4( ↓ 1.0)/ | 94.0( ↓ 5.4)/ | 99.0/ | 98.7( ↓ 0.7)/ | 98.6( ↓ 0.8)/ | 99.3/ | 99.1/ | 99.1/ |
| | 0.5/0.1 | 1.1/0.5 | 5.2/0.8 | 0.8/0.2 | 1.0/0.2 | 1.1/0.3 | 0.6/0.0 | 0.7/0.1 | 0.8/0.1 |
| F03 | 99.1/ | 96.2(↓ 2.9)/ | 95.4( ↓ 3.7)/ | 98.8/ | 98.5( ↓ 0.6)/ | 98.3( ↓ 0.8)/ | 99.2/ | 99.0/ | 99.0/ |
| | 0.8/0.1 | 3.3/0.5 | 3.8/0.8 | 1.0/0.2 | 1.2/0.3 | 1.3/0.4 | 0.8/0.0 | 0.9/0.1 | 0.9/0.1 |
| F04 | 98.2/ | 91.3( ↓ 6.9)/ | 85.7( ↓ 12.5)/ | 97.8/ | 97.7/ | 97.3( ↓ 0.9)/ | 98.3/ | 98.4/ | 98.0/ |
| | 1.7/0.1 | 8.2/0.5 | 13.5/0.8 | 2.0/0.2 | 2.0/0.4 | 2.3/0.4 | 1.6/0.1 | 1.5/0.1 | 1.9/0.1 |
| F05 | 99.2/ | 94.9( ↓ 4.3)/ | 92.1( ↓ 7.1)/ | 99.0/ | 98.8/ | 98.6( ↓ 0.6)/ | 99.2/ | 99.1/ | 99.1/ |
| | 0.7/0.1 | 4.6/0.5 | 7.0/0.8 | 0.8/0.2 | 1.0/0.2 | 1.1/0.3 | 0.7/0.1 | 0.8/0.1 | 0.8/0.1 |
| F06 | 98.9/ | NT/NT/NT | NT/NT/NT | 98.0(↓ 0.9)/ | 97.4( ↓ 1.5)/ | 97.0( ↓ 1.9)/ | 99.1/ | 98.8/ | 98.8/ |
| | 1.0/0.1 | | | 1.8/0.2 | 2.2/0.4 | 2.6/0.5 | 0.8/0.1 | 1.1/0.1 | 1.1/0.1 |

Abbreviation: HMW = High molecular weight; LMW = Low molecular weight; NT = Not test; W = Week; M = Month.

TABLE 32
Formulation feasibility study results: iCIEF.

| | iCIEF (Main peak %/Acidic peaks %/Basic peaks %) | | | | | | | | |
|---|---|---|---|---|---|---|---|---|---|
| | | 40° C. | | 25° C. | | | 5° C. | | |
| No. | T0 | 2 W | 4 W | 1 M | 2 M | 3 M | 1 M | 3 M | 6 M |
| F01 | 36.3/ | 17.1(↓19.2)/ | 11.4(↓24.9)/ | 26.0(↓10.3)/ | 20.2(↓16.1)/ | 16.3(↓20.0)/ | 35.6(↓0.7)/ | 33.7(↓2.6)/ | 29.3(↓7.0)/ |
| | 56.5/7.3 | 75.4/7.5 | 82.7/5.9 | 65.4/8.6 | 71.2/8.6 | 77.4/6.3 | 57.8/6.6 | 59.5/6.9 | 63.3/7.4 |
| F02 | 36.7/ | 16.3(↓20.4)/ | 10.7(↓26.0)/ | 25.7(↓11.0)/ | 19.7(↓17.0)/ | 15.4(↓21.3)/ | 35.8(↓0.9)/ | 33.4(↓3.3)/ | 30.3(↓6.4)/ |
| | 56.1/7.2 | 74.5/9.2 | 80.3/9.0 | 65.3/9.0 | 72.5/7.8 | 78.8/5.7 | 58.0/6.2 | 60.3/6.3 | 62.6/7.0 |
| F03 | 36.2/ | 14.5(↓21.7)/ | 9.0(↓27.2)/ | 24.6(↓11.6)/ | 19.5(↓16.7)/ | 16.2(↓20.0)/ | 35.3(↓0.9)/ | 32.5(↓3.7)/ | 30.9(↓5.3)/ |
| | 56.1/7.7 | 67.9/17.6 | 71.3/19.8 | 66.5/8.8 | 71.6/8.8 | 78.3/5.5 | 57.8/6.9 | 61.1/6.4 | 62.3/6.7 |
| F04 | 36.8/ | 14.3(↓22.5)/ | 7.6(↓29.2)/ | 23.4(↓13.4)/ | 15.9(↓20.9)/ | 13.9(↓22.9)/ | 34.4(↓2.4)/ | 33.1(↓3.7)/ | 28.4(↓8.4)/ |
| | 55.5/7.7 | 74.3/11.5 | 73.9/18.5 | 67.6/9.1 | 75.3/8.8 | 79.5/6.6 | 57.9/7.7 | 60.4/6.5 | 64.1/7.6 |
| F05 | 36.7/ | 17.8(↓18.9)/ | 9.7(↓27.0)/ | 26.6(↓10.1)/ | 21.0(↓15.7)/ | 17.5(↓19.2)/ | 35.7(↓1.0)/ | 34.4(↓2.3)/ | 30.9(↓5.8)/ |
| | 55.9/7.4 | 69.8/12.5 | 66.4/23.9 | 64.4/9.0 | 69.8/9.2 | 74.9/7.6 | 57.0/7.4 | 59.1/6.5 | 62.2/6.9 |
| F06 | 38.5/ | NT/NT/NT | NT/NT/NT | 35.6(↓2.9)/ | 33.3(↓5.2)/ | 31.1(↓7.4)/ | 38.7/ | 39.3/ | 40.2/ |
| | 53.9/7.6 | | | 47.7/16.7 | 44.1/22.6 | 43.5/25.4 | 53.7/7.6 | 53.1/7.5 | 49.8/9.9 |

Caliper-SDS-NR & R: Caliper-SDS-NR & R testing results for the formulation feasibility study are reported in Table 33. For Caliper-SDS-NR, the purity of all samples decreased with the increase of incubation time at 40° C. and 25° C., and the decrease in purity was up to 4.2% and 3.2% when incubated at 40° C. for 4 weeks and 25° C. for 3 months, respectively The formulation with 0.9% benzyl alcohol performed comparable to the corresponding formulation without benzyl alcohol within the expected variability of the analytical method. For Caliper-SDS-R, less changes were seen in all the samples compared with Caliper-SDS-NR results, indicating that breakage of inter-chain disulfide bonds might occur during incubation at 40° C. Slight decrease in purity at 40° C. for up to 4 weeks and at 25° C. for up to 3 months was observed, there was no significant decline observed in the 5° C. samples. All formulations were stable at the control recommended storage condition of 5° C. for up to 6 months.

TABLE 33
Formulation feasibility study results: Caliper-SDS-NR & R.

| | | | | | | | | | | Caliper- |
|---|---|---|---|---|---|---|---|---|---|---|
| | Caliper-SDS-NR (Purity) % | | | | | | | | | SDS-R |
| | | 40° C. | | 25° C. | | | 5° C. | | | (Purity) % |
| No. | T0 | 2 W | 4 W | 1 M | 2 M | 3 M | 1 M | 3 M | 6 M | T0 |
| F01 | 94.9 | 94.1 | 93.1 | 96.0 | 95.1 | 94.4 | 96.3 | 95.8 | 95.8 | 99.2 |
| | | ( ↓ 0.8) | ( ↓ 1.8) | | | ( ↓ 0.5) | | | | |
| F02 | 96.1 | 94.4 | 93.1 | 96.0 | 95.2 | 94.5 | 96.4 | 95.6 | 95.6 | 99.3 |
| | | ( ↓ 1.7) | ( ↓ 3.0) | | ( ↓ 0.9) | ( ↓ 1.6) | | | | |
| F03 | 96.0 | 94.0 | 93.1 | 96.0 | 95.3 | 94.5 | 96.4 | 95.7 | 95.9 | 99.2 |
| | | ( ↓ 2.0) | ( ↓ 2.9) | | ( ↓ 0.7) | ( ↓ 1.5) | | | | |
| F04 | 96.4 | 93.5 | 92.2 | 95.5 | 94.4 | 93.2 | 96.2 | 95.6 | 95.6 | 99.2 |
| | | ( ↓ 2.9) | ( ↓ 4.2) | ( ↓ 0.9) | ( ↓ 2.0) | ( ↓ 3.2) | | | | |
| F05 | 96.3 | 94.5 | 93.7 | 96.3 | 95.5 | 94.8 | 96.2 | 95.7 | 95.8 | 99.2 |
| | | ( ↓ 1.8) | ( ↓ 2.6) | | ( ↓ 0.8) | ( ↓ 1.5) | | | | |
| F06 | 96.4 | NT | NT | 96.0 | 95.3 | 94.5 | 96.6 | 95.8 | 95.7 | 99.2 |
| | | | | ( ↓ 0.4) | ( ↓ 1.1) | ( ↓ 1.9) | | | | |
| | | | Caliper-SDS-R (Purity) % | | | | | | | |
| | | | 40° C. | | 25° C. | | | 5° C. | | |
| | | No. | 2 W | 4 W | 1 M | 2 M | 3 M | 1 M | 3 M | 6 M |
| | | F01 | 97.6 | 97.3 | 98.8 | 98.4 | 97.2 | 98.9 | 97.6 | 98.2 |
| | | | ( ↓ 1.6) | ( ↓ 1.9) | ( ↓ 0.4) | ( ↓ 0.8) | ( ↓ 2.0) | | | |
| | | F02 | 97.6 | 96.9 | 98.7 | 98.3 | 97.7 | 99.0 | 98.1 | 98.2 |
| | | | ( ↓ 1.7) | ( ↓ 2.4) | ( ↓ 0.6) | ( ↓ 1.0) | ( ↓ 1.6) | | | |
| | | F03 | 97.4 | 96.8 | 98.8 | 98.1 | 97.5 | 98.9 | 97.8 | 98.2 |
| | | | ( ↓ 1.8) | ( ↓ 2.4) | ( ↓ 0.4) | ( ↓ 1.1) | ( ↓ 1.7) | | | |
| | | F04 | 97.5 | 97.0 | 98.8 | 98.2 | 97.3 | 98.9 | 97.6 | 98.2 |
| | | | ( ↓ 1.7) | ( ↓ 2.2) | ( ↓ 0.4) | ( ↓ 1.0) | ( ↓ 1.9) | | | |
| | | F05 | 97.4 | 97.0 | 98.8 | 98.1 | 97.6 | 98.9 | 97.9 | 98.0 |
| | | | ( ↓ 1.8) | ( ↓ 2.2) | ( ↓ 0.4) | ( ↓ 1.1) | ( ↓ 1.6) | | | |
| | | F06 | NT | NT | 98.6 | 98.0 | 97.1 | 99.0 | 96.3 | 98.2 |
| | | | | | ( ↓ 0.6) | ( ↓ 1.2) | ( ↓ 2.1) | | | |

Sub-visible particle (HIAC): The sub-visible particles (HIAC) testing results for the formulation feasibility study are shown in Table 34. After incubated at different thermal conditions, no obvious changes in amount of ≥2 μm, ≥10 μm, ≥25 μm sub-visible particles were observed. All formulations were stable at the control recommended storage condition of 5° C. for up to 6 months.

TABLE 34
Formulation feasibility study results: sub-visible particles (HIAC).

| | Sub-visible particles counts (#/ml, ≥2 μm/≥10 μm/≥25 μm) | | | | | | |
|---|---|---|---|---|---|---|---|
| | | 40° C. | | 25° C. | | 5° C. | |
| No. | T0 | 2 W | 4 W | 2 M | 3 M | 3 M | 6 M |
| F01 | 602/139/86 | 361/16/0 | 555/25/1 | 671/20/0 | 524/15/0 | 760/7/0 | 799/5/0 |
| F02 | 49/6/1 | 353/4/0 | 351/8/0 | 220/5/0 | 231/5/0 | 285/0/0 | 522/5/0 |
| F03 | 263/14/0 | 310/18/2 | 247/6/0 | 311/12/0 | 238/2/1 | 578/5/0 | 1153/9/0 |
| F04 | 801/4/1 | 99/5/0 | 180/6/1 | 169/2/0 | 399/2/0 | 289/2/0 | 105/3/0 |
| F05 | 69/6/1 | 35/2/0 | 78/5/0 | 167/2/0 | 142/1/0 | 281/2/0 | 445/5/0 |
| F06 | 42/6/1 | NT/NT/NT | NT/NT/NT | 136/3/0 | 370/0/0 | 211/0/0 | 302/4/0 |

Abbreviation: W = Week; M = Month; NT = Not test.

Potency: Based on the above testing results, F02 (60 mg/mL protein, 20 mM Histidine, 500 (w/v) sorbitol, 0.900 (w/v) benzyl alcohol, at pH 6.6), F03 (60 mg/mL protein, 20 mM Histidine, 50% (w/v) sorbitol, 1.2% (w/v) benzyl alcohol, at pH 6.6) and F05 (30 mg/mL protein, 20 mM Histidine, 9% (w/v) sucrose, 1.2% (w/v) benzyl alcohol, at pH 6.6) were selected for potency testing. As shown in Table 35, no difference in potency was found when IVX-01286 protein was stored at 5° C. for 6 months compared to T0 within the acceptance criteria of 50-150% based on analytical method variability.

TABLE 35
Formulation feasibility study results: ELISA

| | | Potency (% ) | |
|---|---|---|---|
| No. | Formulation | T0 | 5° C.-6 M |
| F01 | 60, His, 6.6, 5% Sorb | NT | NT |
| F02 | 60, His, 6.6, 5% Sorb, 0.9% BA | 94 | 88 |
| F03 | 60, His, 6.6, 5% Sorb, 1.2% BA | NT | 88 |
| F04 | 60, His, 6.6, 5% Sorb, 0.4% CR | NT | NT |
| F05 | 30, His, 6.6, 9% Suc, 1.2% BA | NT | 81 |
| F06 | 60, Ace, 5.0, 9% Suc, 1.2% BA | NT | NT |

Abbreviation:
W = Week;
M = Month;
NT = Not test;
His = Histidine;
Ace = Acetate;
Sorb = Sorbitol;
BA = Benzyl alcohol;
CR = m-cresol;
Suc = Sucrose;
NT = Not test.

Summary of Formulation Feasibility Study: The formulation feasibility study investigated the stability of IVX-01286 molecule formulated in different formulations with varying concentrations of preservatives (antimicrobial agent)s under stress conditions (40° C. for up to 4 weeks, 25° C. for up to 3 months) and long-term storage condition (5° C. for up to 6 months). The results showed that stability of the IVX-01286 molecule was closely related with the type and strengths of antimicrobials. The concentration of 0.4% (w/v) m-cresol had more impact than 1.2% (w/v) benzyl alcohol on protein quality when at the same protein concentration.

Based on the study results, IVX-01286 molecule formulated in 20 mM Histidine buffer, 5% (w/v) sorbitol, 0.02% (w/v) PS80, 0.05 mM EDTA, 0.9% (w/v) benzyl alcohol, pH 6.6 showed relatively good thermal stability and stability profile is similar to the molecule in the formulation without preservative, i.e. 20 mM Histidine buffer, 5% (w/v) sorbitol, 0.02% (w/v) PS80, 0.05 mM EDTA, pH 6.6.

### Example 4: IVX-03023 Formulation Feasibility Study

### Materials and Methods

Development batch of IVX-03023 Feline monoclonal antibody (mAb) (also referred to as IVX-03) was produced at a 15 L bioreactor scale for use in proof of concept studies using the pool of clones generated by stable expression system. 10 mg/mL of IVX-03023 mAb was formulated in 20 mM acetic acid and sodium acetate buffer, 9% (w/w) Sucrose, 0.02% (w/w) Polysorbate 80 at pH 5.0 for Drug substance (DS) and Drug Product (DP) material. Product stability was monitored using multiple analytical methods including SE-HPLC, Reduced and Non-Reduced capillary electrophoresis-SDS (CE-SDS) Imaged capillary isoelectric focusing (iCIEF) concentration and potency, as described above. Sub-visible particles was also monitored for DP. All method information are similar to what has been described above. Only the ELISA method is different as described below.

ELISA Potency Assay: The IL5 antigen is coated on the surface of the wells of a 96-well plate to capture the IVX-03023 antibody. After the binding of IVX-03023 antibody, a goat anti-feline IgG (H+L) secondary antibody fused to the HRP enzyme for detection is added to bind to IVX-03023 antibody. Addition of the TMB substrate results in color development in the assay wells. After adding stop solution, the assay plate is read at 450 nm and 630 nm. The OD difference between 450 nm and 630 nm is proportional to the quantity of bound IVX-03023. Standard curves are plotted and fit according to a 4-parameter logistic regression model using the SoftMax Pro Software for samples and reference standard, which are used to calculate EC50 values for the curves. The conversion of EC50 for the samples to binding potency is calculated by comparison to the reference standard assayed on the same plate.

### Results

The following is long-term stability data for the IVX-03023 monoclonal antibody with a drug product formulation of 20 mM NaAc—HAc, 9% (w/w) Sucrose, 0.02% (w/w) PS80, pH 5.0, 10 mg/mL mAb. 15 L Bioreactor non-GMP Stable Pool Production Stability were conducted

The data summary (DS) information used in the −70° C.±10° C. IVX-03023 formulation development study is listed in Table 36.

Study Parameters: The sampling and testing plan for the formulation feasibility study are shown below. For thermal stress, samples were stored at −70° C.±10° C. for up to 12 months (Table 36), 5° C. for up to 12 months (Table 37), 25° C. for up to 3 months (Table 38), 40° C.±2° C. for up to 1 month (Table 39). Testing items including appearance, protein concentration, ELISA binding, pH, CE-SDS (reduced and non-reduced), SE-UPLC, and iCIEF.

TABLE 36
Study Parameters for IVX-03023 (−70° C. ± 10° C.)

| Test/Time | Specification/ | T = 0 | T = 1 M | T = 3 M |
|---|---|---|---|---|
| point | Acceptance Criteria | (2022 Oct. 26) | (2022 Nov. 26) | (2023 Jan. 26) |
| Color | Colorless to slight | Colorless | Colorless | Colorless |
| | yellow | | | |
| Clarity | Clear to slightly | Clear | Clear | Clear |
| | opalescent solution | | | |
| Protein | 10 ± 1 mg/mL | 10.4 | 10.4 | 10.5 |
| Concentration | | | | |
| ELISA | 50-150% RP | 96.89% | NA | NA |
| Binding | | | | |
| pH | 5.0 ± 0.5 | 5.1 | 5.0 | 5.1 |
| CE-SDS | Heavy + Light Chain | % Impurity = 1.9 | % Impurity = 3.3 | % Impurity = 3.8 |
| (reduced) | Peak Area ≥ 90% | % Light Chain = 26.9 | % Light Chain = 26.4 | % Light Chain = 26.9 |
| | | % Heavy Chain = 71.2 | % Heavy Chain = 70.3 | % Heavy Chain = 69.3 |
| | | % Purity | % Purity | % Purity |
| | | (% LC + % HC) = 98.1 | (% LC + % HC) = 96.7 | (% LC + % HC) = 96.2 |
| CE-SDS | Main Peak | %Pre-Peaks = 4.0 | % Pre-Peaks = 4.9 | % Pre-Peaks = 4.0 |
| (non-reduced) | Purity ≥ 90% | %Main Peak = 96.0 | % Main Peak = 95.1 | % Main Peak = 96.0 |
| SEC-UPLC | Monomer: ≥90% | Monomer: 99.4% | Monomer: 99.4% | Monomer: 99.4% |
| | HMW: Report Results | HMW: 0.6% | HMW: 0.6% | HMW: 0.6% |
| | LMW: Report | LMW: ND² | LMW: ND | LMW: ND |
| | Results | | | |
| iCIEF | Main Species: Report | Main Species: 53.7% | Main Species: 54.3% | Main Species: 54.8% |
| | Results | Acidic Species: 33.9% | Acidic Species:33.9% | Acidic Species: 32.2% |
| | Acidic Species: | Basic Species: 12.5% | Basic Species: 11.8% | Basic Species: 13.0% |
| | Report Result | | | |
| | | Test/Time | T = 6 M | T = 12 M |
| | | point | (2023 Apr. 26) | (2023 Oct. 26) |
| | | Color | Colorless | Colorless |
| | | Clarity | Clear | Clear |
| | | Protein | 10.4 | 10.5 |
| | | Concentration | | |
| | | ELISA | NA | 81.47% |
| | | Binding | | |
| | | pH | 5.1 | 5.1 |
| | | CE-SDS | % Impurity = 3.4 | % Impurity = 3.5 |
| | | (reduced) | % Light Chain = 27.0 | % Light Chain = 27.1 |
| | | | % Heavy Chain = 69.6 | % Heavy Chain = 69.4 |
| | | | % Purity | % Purity |
| | | | (% LC + % HC) = 96.6 | (% LC + % HC) = 96.5 |
| | | CE-SDS | % Pre-Peaks = 3.9 | % Pre-Peaks = 4.5 |
| | | (non-reduced) | % Main Peak = 96.1 | % Main Peak = 95.5 |
| | | SEC-UPLC | Monomer: 99.4% | Monomer: 99.4% |
| | | | HMW: 0.6% | HMW: 0.6% |
| | | | LMW: ND | LMW: ND |
| | | iCIEF | Main Species: 54.9% | Main Species: 54.3% |
| | | | Acidic Species: 31.7% | Acidic Species: 28.1% |
| | | | Basic Species: 13.4% | Basic Species: 17.6% |

TABLE 37
Study Parameters for IVX-03023 (5° C. ± 3° C.)

| Test/Time | Specification/ | T = 0 | | T = 1 M | | T = 3 M | |
|---|---|---|---|---|---|---|---|
| point | Acceptance Criteria | (2022 Oct. 26) | | (2022 Nov. 26) | | (2023 Jan. 26) | |
| Color | Colorless to | Colorless | | Colorless | | Colorless | |
| | slight | | | | | | |
| | yellow | | | | | | |
| Clarity | Clear to | Clear | | Clear | | Clear | |
| | slightly | | | | | | |
| | opalescent | | | | | | |
| | solution | | | | | | |
| Visible | ≤10 Visible | Essentially | | Essentially free | | Essentially free | |
| Particles | Proteinaceous | free of | | of visible | | of visible | |
| | particles; No | visible | | particles | | particles | |
| | foreign particles | particles | | | | | |
| Subvisible | Report Results | ≥2 μm: 108 | | ≥2 μm: 189 | | ≥2 μm: 22 | |
| Particles by | for ≥2 μm and | ≥5 μm: 33 | | ≥5 μm: 97 | | ≥5 μm: 2 | |
| HIAC | ≥5 μm particles. | ≥10 μm: 10 | | ≥10 μm: 34 | | ≥10 μm: 0 | |
| | ≥10 μm: | ≥25 μm: 0 | | ≥25 μm: 2 | | ≥25 μm: 0 | |
| | ≤6000 particles/mL | | | | | | |
| | ≥25 μm: | | | | | | |
| | ≤600 particles/mL | | | | | | |
| Protein | 10 ± 1 mg/mL | 10.4 | | 10.4 | | 10.5 | |
| Concentration | | | | | | | |
| ELISA | 50-150% RP | 84.40% | | NA | | NA | |
| Binding | | | | | | | |
| pH | 5.0 ± 0.5 | 5.1 | | 5.0 | | 5.1 | |
| CE-SDS | Heavy + | % Impurity = 1.4 | | % Impurity = 4.1 | | % Impurity = 4.1 | |
| (reduced) | Light Chain | % Light Chain = 26.6 | | % Light Chain = 26.5 | | % Light Chain = 27.1 | |
| | Peak Area ≥ 90% | % Heavy Chain = 72.1 | | % Heavy Chain = 69.4 | | % Heavy Chain = 68.8 | |
| | | % Purity | | % Purity | | % Purity | |
| | | (% LC + % HC) = 98.6 | | (% LC + % HC) = 96.0 | | (% LC + % HC) = 95.9 | |
| CE-SDS | Main Peak | % Pre-Peaks = 5.9 | | % Pre-Peaks = 4.6 | | % Pre-Peaks = 4.0 | |
| (non-reduced) | Purity ≥ 90% | % Main Peak = 94.2 | | % Main Peak = 95.4 | | % Main Peak = 96.0 | |
| SEC-UPLC | Monomer: | Monomer: 99.3% | | Monomer: 99.3% | | Monomer: 99.1% | |
| | 90% HMW: | HMW: 0.7% | | HMW: 0.7% | | HMW: 0.9% | |
| | Report Results | LMW: ND² | | LMW: ND | | LMW: ND | |
| | LMW: Report | | | | | | |
| | Results | | | | | | |
| iCIEF | Main Species: | Main Species: 51.7% | | Main Species: 54.5% | | Main Species: 54.2% | |
| | Report Results | Acidic Species: 33.6% | | Acidic Species: 32.0% | | Acidic Species: 28.6% | |
| | Acidic Species: | Basic Species: 14.7% | | Basic Species: 13.5% | | Basic Species: 17.2% | |
| | Report Result | | | | | | |
| PS80 | −0.05 to 0.35% | NA | | NA | | NA | |
| | w/v | | | | | | |
| | | Test/Time | | T = 6 M | | T = 12 M | |
| | | point | | (2023 Apr. 26) | | (2023 Oct. 26) | |
| | | Color | | Colorless | | Colorless | |
| | | Clarity | | Clear | | Clear | |
| | | Visible | | Essentially free of | | Essentially free of | |
| | | Particles | | visible particles | | visible particles | |
| | | Subvisible | | ≥2 μm: 242 | | ≥2 μm: 110 | |
| | | Particles by | | ≥5 μm: 15 | | ≥5 μm: 34 | |
| | | HIAC | | ≥10 μm: 4 | | ≥10 μm: 15 | |
| | | | | ≥25 μm: 0 | | ≥25 μm: 0 | |
| | | Protein | | 10.4 | | 10.5 | |
| | | Concentration | | | | | |
| | | ELISA | | NA | | 86.47% | |
| | | Binding | | | | | |
| | | pH | | 5.1 | | 5.1 | |
| | | CE-SDS | | % Impurity = 3.9 | | % Impurity = 4.0 | |
| | | (reduced) | | % Light Chain = 26.9 | | % Light Chain = 28.1 | |
| | | | | % Heavy Chain = 69.2 | | % Heavy Chain = 67.9 | |
| | | | | % Purity | | % Purity | |
| | | | | (% LC + % HC) = 96.1 | | (% LC + % HC) = 96.0 | |
| | | CE-SDS | | % Pre-Peaks = 4.6 | | % Pre-Peaks = 4.1 | |
| | | (non-reduced) | | % Main Peak = 95.4 | | % Main Peak = 95.9 | |
| | | SEC-UPLC | | Monomer: 99.0% | | Monomer: 98.8% | |
| | | | | HMW: 1.0% | | HMW: 1.2% | |
| | | | | LMW: ND | | LMW: ND | |
| | | iCIEF | | Main Species: 51.8% | | Main Species: 47.7% | |
| | | | | Acidic Species: 27.7% | | Acidic Species: 27.8% | |
| | | | | Basic Species: 20.5% | | Basic Species: 24.6% | |
| | | PS80 | | 0.02% | | 0.02% | |
| Stability | 25° C. ± 2° C.- | Timepoint | T = 1, 3 M | Stability | 2022 Oct. 26 | Stability | CRAN- |
| Condition | Invented 60 | | | Start Date | | Protocol No. | WBP2470- |
| | RH ± 5% RH | | | | | | DPD-STP- |
| | | | | | | | 002-01 |

TABLE 38
Study Parameters for IVX-03023 (25° C. ± 2° C.)

| Test/ | Specification/ | T = 0 | T = 1 M | T = 3 M |
|---|---|---|---|---|
| Time point | Acceptance Criteria | (2022 Oct. 26) | (2022 Nov. 26) | (2022 Jan. 26) |
| Color | Colorless to slight yellow | Colorless | Colorless | Colorless |
| Clarity | Clear to slightly opalescent | Clear | Clear | Clear |
| | solution | | | |
| Visible | ≤10 Visible Proteinaceous | Essentially free of | Essentially free of | Essentially free of |
| Particles | particles; No foreign | visible particles | visible particles | visible particles |
| | particles | | | |
| Subvisible | Report Results for ≥2 μm | ≥2 μm: 108 | ≥2 μm: 597 | ≥2 μm: 349 |
| Particles by | and ≥5 μm particles. | ≥5 μm: 33 | ≥5 μm: 155 | ≥5 μm: 63 |
| HIAC | ≥10 μm: ≤6000 particles/mL | ≥10 μm: 10 | ≥10 μm: 17 | ≥10 μm: 13 |
| | ≥25 μm: ≤600 particles/mL | ≥25 μm: 0 | ≥25 μm: 0 | ≥25 μm: 0 |
| Protein | 10 ± 1 mg/mL | 10.4 | 10.5 | 10.5 |
| Concentration | | | | |
| pH | 5.0 ± 0.5 | 5.1 | 5.0 | 5.1 |
| CE-SDS | Heavy + Light | % Impurity = 1.4 | % Impurity = 2.9 | % Impurity = 4.2 |
| (reduced) | Chain Peak | % Light Chain = 26.6 | % Light Chain = 26.1 | % Light Chain = 27.3 |
| | Area ≥90% | % Heavy Chain = 72.1 | % Heavy Chain = 71.1 | % Heavy Chain = 68.6 |
| | | % Purity(% LC + | % Purity(% LC + | % Purity(% LC + |
| | | % HC) = 98.6 | % HC) = 97.2 | % HC) = 95.8 |
| CE-SDS | Main Peak Purity ≥90% | % Pre-Peaks = 5.9 | % Pre-Peaks = 5.1 | % Pre-Peaks = 3.7 |
| (non-reduced) | | % Main Peak = 94.2 | % Main Peak = 94.9 | % Main Peak = 96.3 |
| SEC-UPLC | Monomer: ≥90% | Monomer: 99.3% | Monomer: 98.9% | Monomer: 98.3% |
| | HMW: Report Results | HMW: 0.7% | HMW: 1.1% | HMW: 1.4% |
| | LMW: Report Results | LMW: ND | LMW: ND | LMW: 0.3% |
| iCIEF | Main Species: Report Results | Main Species: 51.7% | Main Species: 50.1% | Main Species: 46.6% |
| | Acidic Species: Report Result | Acidic Species: 33.6% | Acidic Species: 30.0% | Acidic Species: 27.1% |
| | Basic Species: Report Result | Basic Species: 14.7% | Basic Species: 19.9% | Basic Species: 26.4% |

TABLE 39
Study Parameters for IVX-03023 (40° C. ± 2 ° C.)

| Test/ | Specification/ | T = 0 | T = 2 wk | T = 1 M |
|---|---|---|---|---|
| Time point | Acceptance Criteria | (2022 Oct. 26) | (2022 Nov. 09) | (2022 Nov. 26) |
| Color | Colorless to slight yellow | Colorless | Colorless | Colorless |
| Clarity | Clear to slightly opalescent | Clear | Clear | Clear |
| | solution | | | |
| Visible | ≤10 Visible Proteinaceous | Essentially free of | Essentially free of | Essentially free |
| Particles | particles; No foreign | visible particles | visible particles | of visible particles |
| | particles | | | |
| Subvisible | Report Results for ≥2 μm | ≥2 μm: 108 | ≥2 μm: 149 | ≥2 μm: 147 |
| Particles by | and ≥5 μm particles. | ≥5 μm: 33 | ≥5 μm: 60 | ≥5 μm: 49 |
| HIAC | ≥10 μm: ≤6000 particles/mL | ≥10 μm: 10 | ≥10 μm: 25 | ≥10 μm: 17 |
| | ≥25 μm: ≤600 particles/mL | ≥25 μm: 0 | ≥25 μm: 10 | ≥25 μm: 0 |
| Protein | 10 ± 1 mg/mL | 10.4 | 10.5 | 10.5 |
| Concentration | | | | |
| pH | 5.0 ± 0.5 | 5.1 | 5.1 | 5.0 |
| CE-SDS | Heavy + Light | % Impurity = 1.4 | % Impurity = 2.7 | % Impurity = 3.2 |
| (reduced) | Chain Peak | % Light Chain = 26.6 | % Light Chain = 26.1 | % Light Chain = 26.1 |
| | Area ≥90% | % Heavy Chain = 72.1 | % Heavy Chain = 71.2 | % Heavy Chain = 70.7 |
| | | % Purity(% LC + | % Purity(% LC + | % Purity(% LC + |
| | | % HC) = 98.6 | % HC) = 97.3 | % HC) = 96.8 |
| CE-SDS | Main Peak Purity ≥90% | % Pre-Peaks = 5.9 | % Pre-Peaks = 5.8 | % Pre-Peaks = 9.8 |
| (non-reduced) | | % Main Peak = 94.2 | % Main Peak = 94.2 | % Main Peak = 90.2 |
| SEC-UPLC | Monomer: ≥90% | Monomer: 99.3% | Monomer: 97.6% | Monomer: 93.2% |
| | HMW: Report Results | HMW: 0.7% | HMW: 1.9% | HMW: 3.2% |
| | LMW: Report Results | LMW: ND² | LMW: 0.5% | LMW: 3.6% |
| iCIEF | Main Species: Report Results | Main Species: 51.7% | Main Species: 47.3% | Main Species: 46.4% |
| | Acidic Species: Report Result | Acidic Species: 33.6% | Acidic Species: 29.0% | Acidic Species: 29.3% |
| | Basic Species: Report Result | Basic Species: 14.7% | Basic Species: 23.7% | Basic Species: 24.4% |
| ELISA | 50-150% RP | 84.40% | NA | 72.79 |
| Binding | | | | |

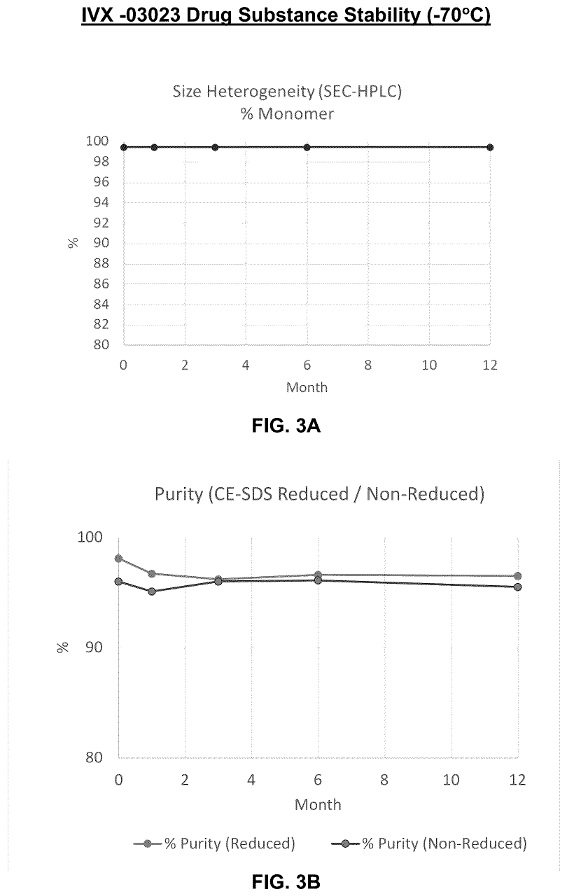

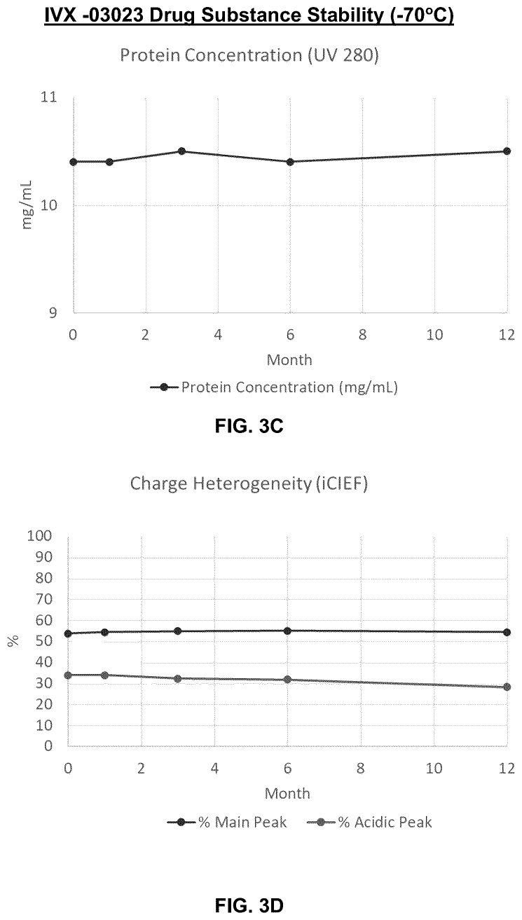

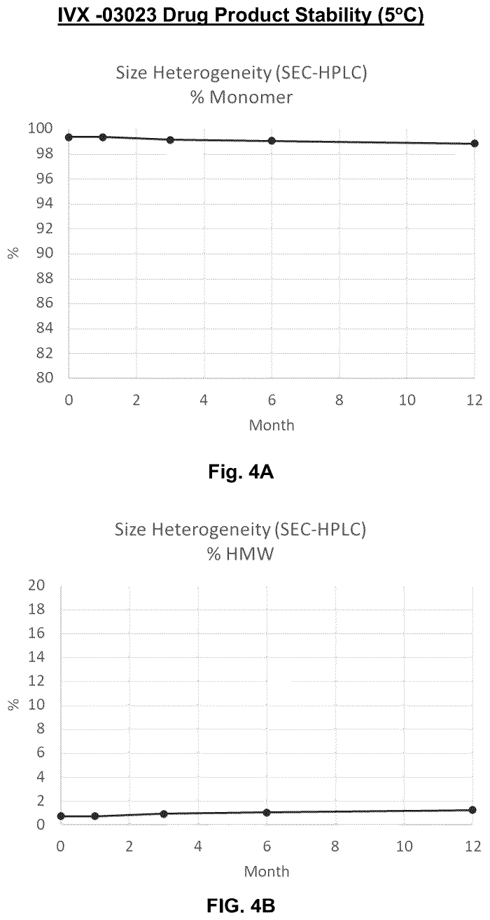

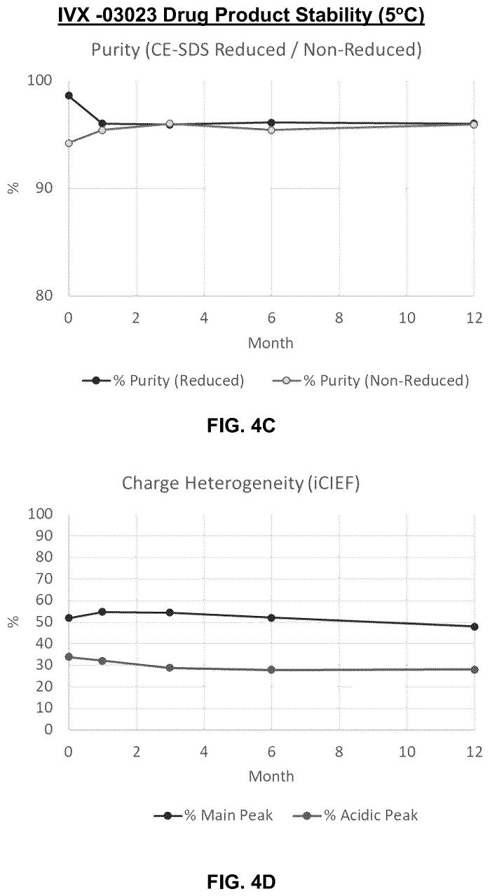

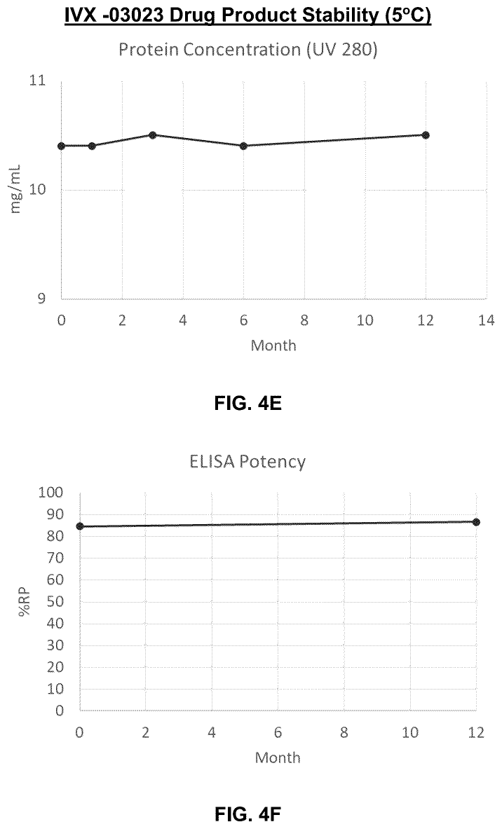

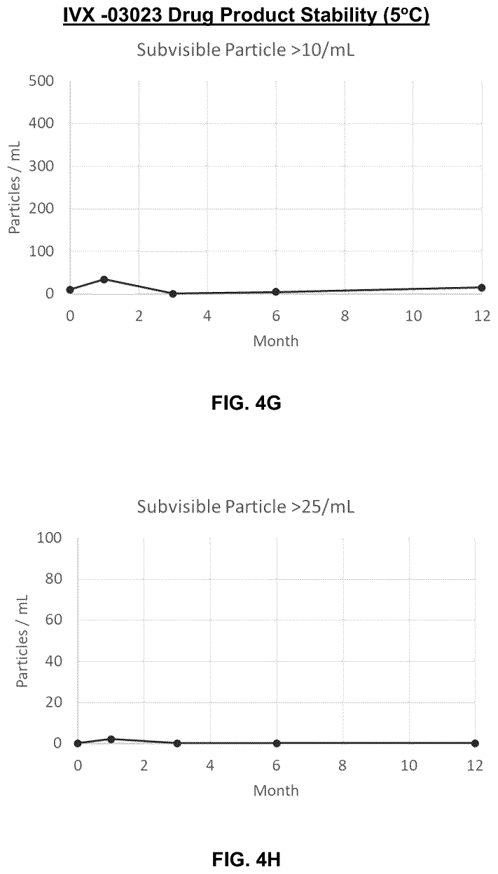

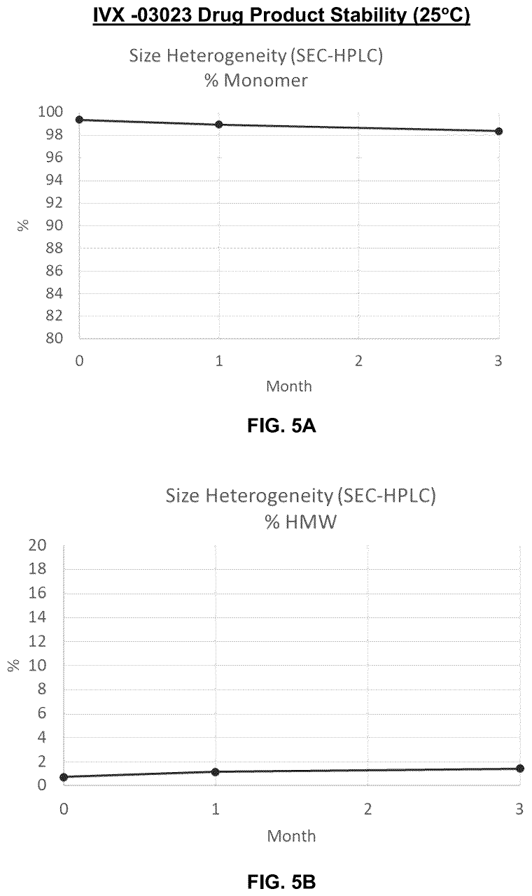

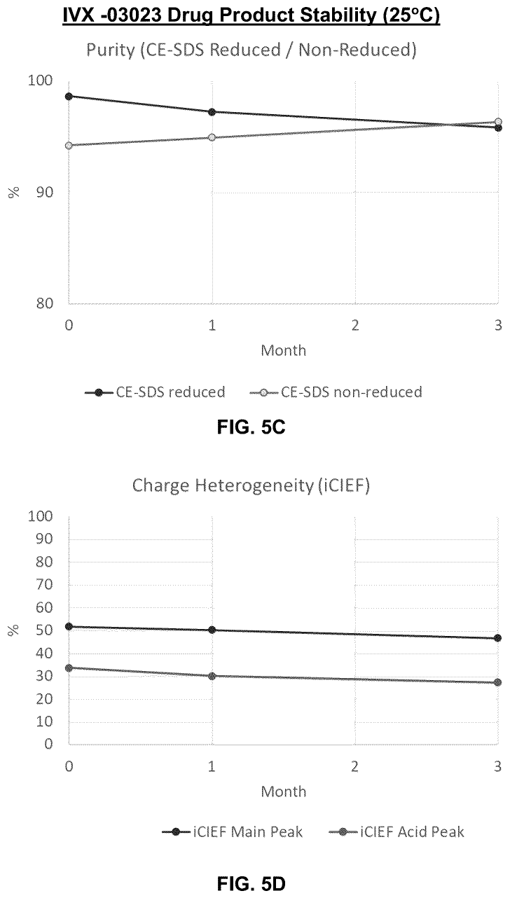

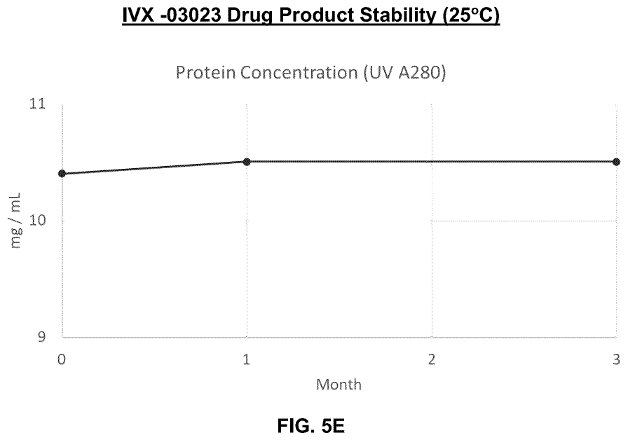

IVX-03023 drug substance was stable at recommended long term storage temperature of −70±−10° C. for 1 year and well above the acceptance criteria for all the product quality attributes (Table 36, FIGS. 3A-3D). The drug product was stable at recommended long term storage temperature of 2-8° C. for 1 year (Table 37, FIGS. 4A-4H) and accelerated temperature of 25° C. for 3 months (Table 48, FIGS. 5A-5E) well above the acceptance criteria for all the product quality attributes. Potency of the molecule is maintained. pH, visible particles and osmolarity results all show no meaningful differences over time.

### Example 5: IVX-06076 Formulation Feasibility Study

### Materials and Methods

Development batch of IVX-06076 Feline mAb (also referred to as IVX-06) was produced at 15 L bioreactor scale for use in proof of concept studies using the pool of clones generated by stable expression system. 10 mg/mL of IVX-06076 mAb was formulated in 20 mM Acetic acid and sodium acetate buffer, 9% (w/w) Sucrose, 0.02% (w/w) Polysorbate 80 at pH 5.0 for Drug substance (DS) and Drug Product (DP) material. Product stability was monitored using multiple analytical methods including SE-HPLC, Reduced and Non-Reduced capillary electrophoresis-SDS (CE-SDS) Imaged capillary isoelectric focusing (iCIEF) concentration and potency. Sub-visible particles was also monitored for DP. All methods are as shown above. ELISA is identical to what is presented for IVX-01101.

### Results

The following is long-term stability data for the IVX-00676 monoclonal antibody with a drug product formulation of 20 mM NaAc-Hac, 9% (w/w) Sucrose, 0.02% (w/w) PS80, pH 5.0, 10 mg/mL mAb. 15 L Bioreactor non-GMP Stable Pool Production Stability were conducted.

The data summary (DS) information used in the −70° C.±10° C. IVX-06076 formulation development study is listed in Table 44.

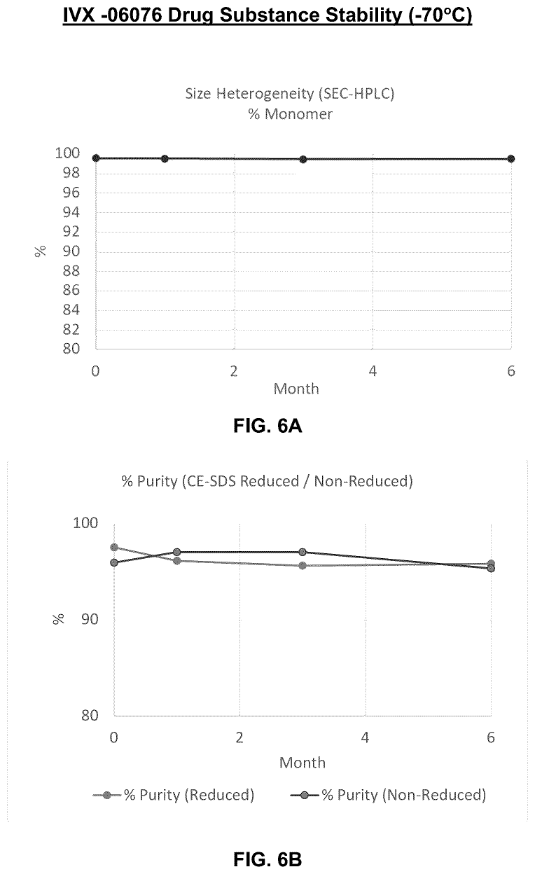

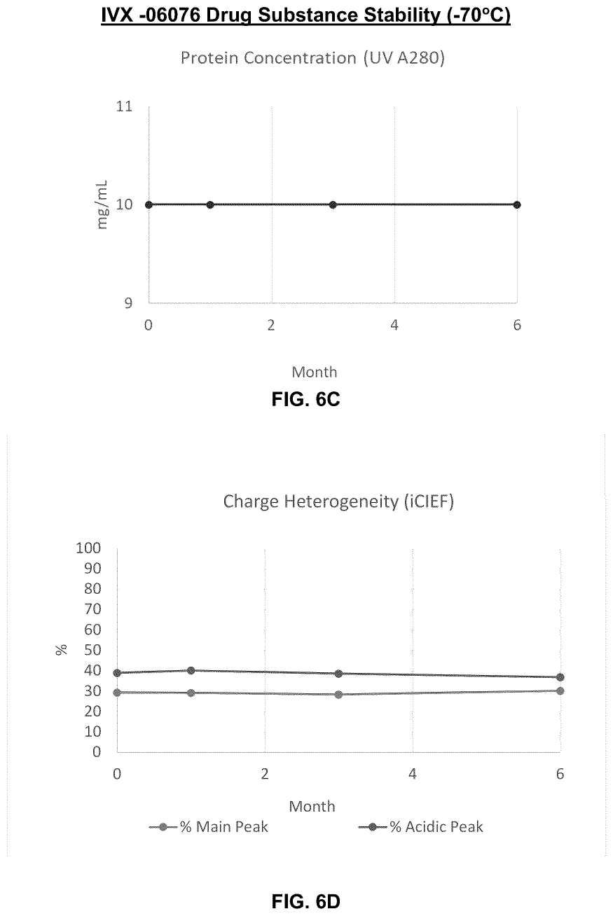

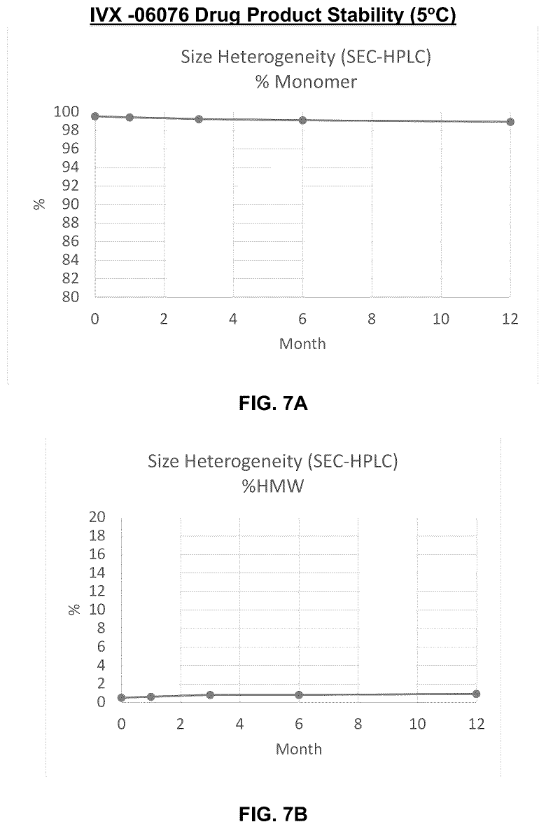

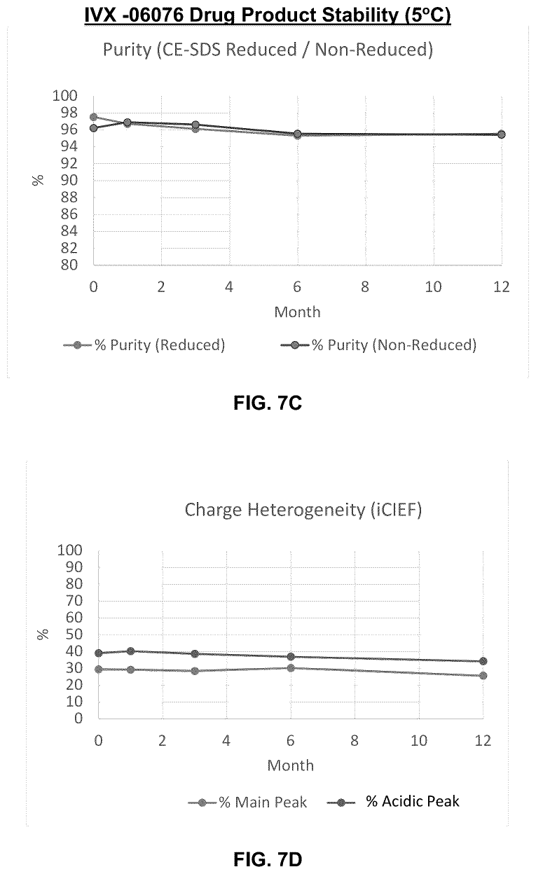

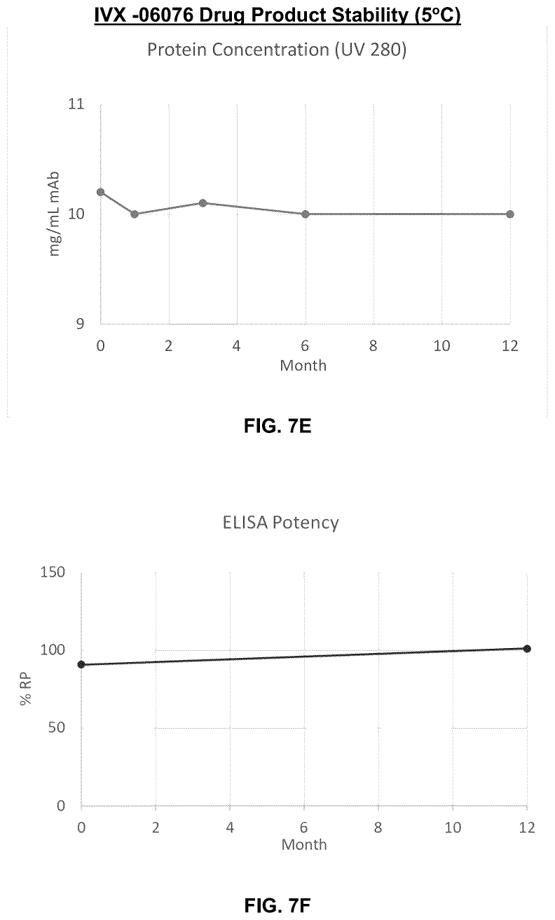

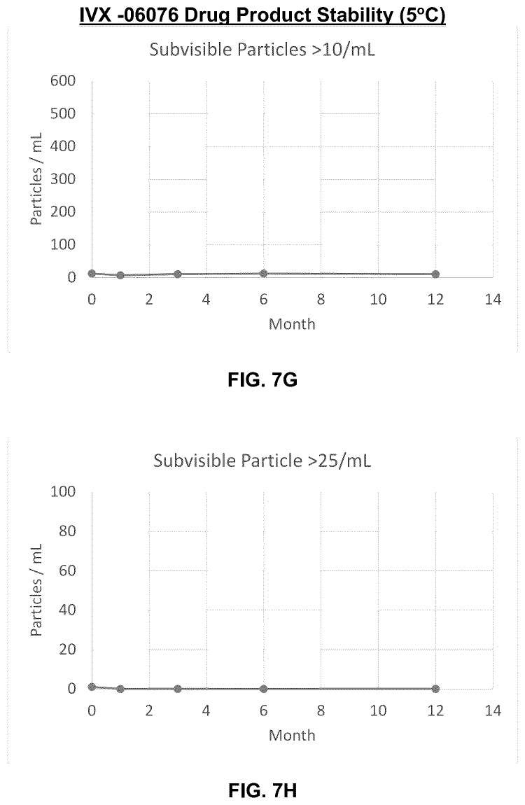

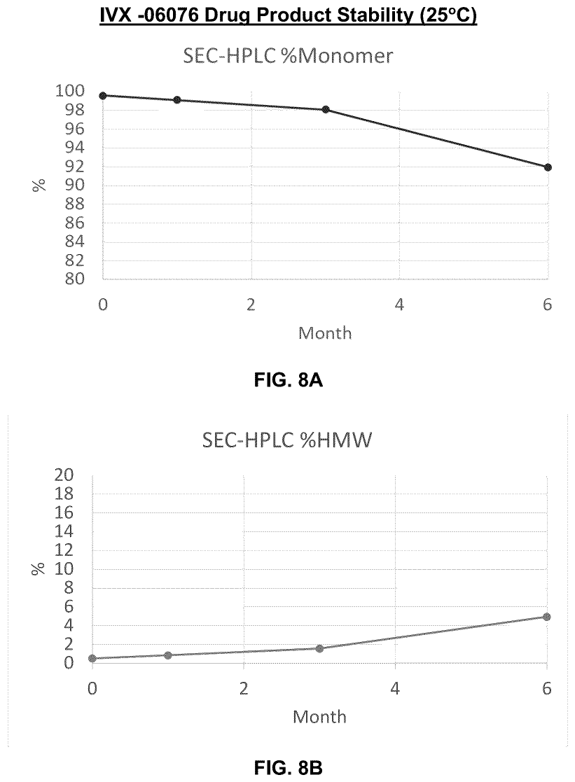

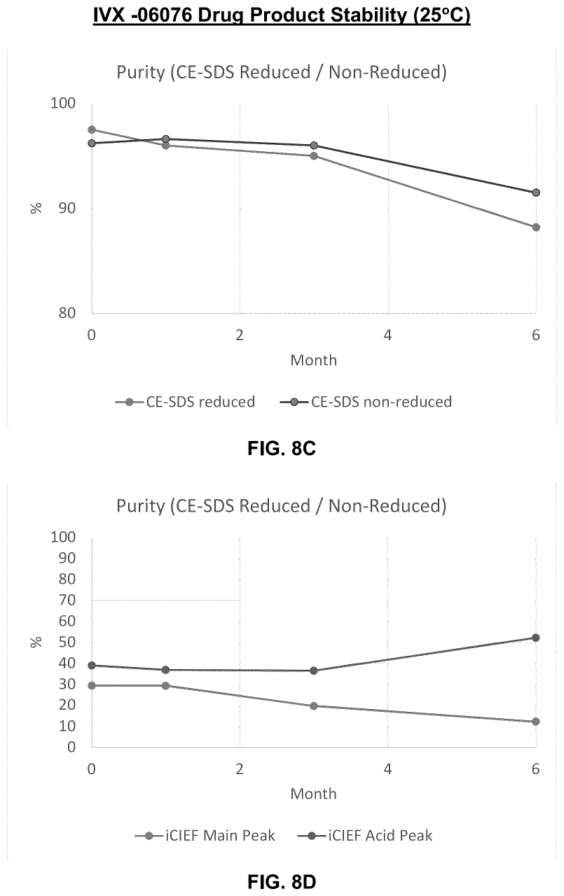

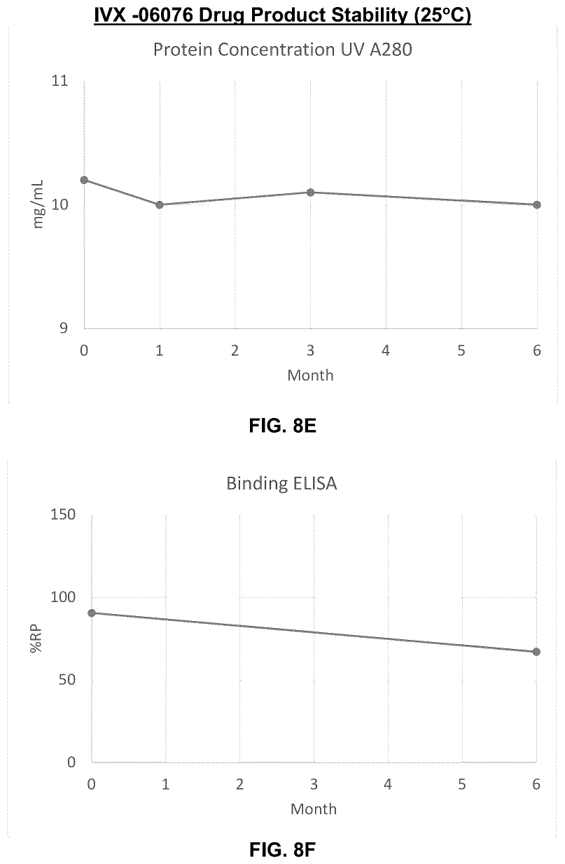

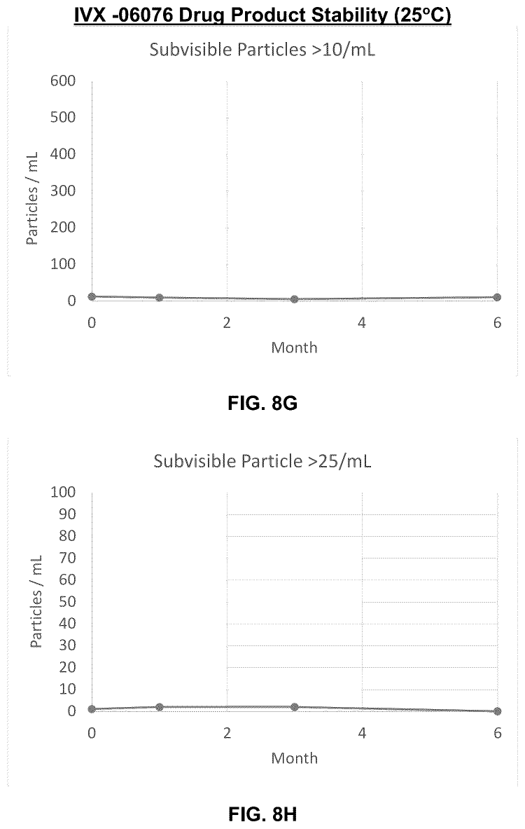

Study Parameters: The sampling and testing plan for the formulation feasibility study for IVX-06076 are shown below. For thermal stress, samples were stored at −70° C.±10° C. for up to 12 months (Table 40, FIGS. 6A-6D), 5° C. for up to 18 months (Table 41, FIGS. 7A-7H), 25° C. for up to 3 months (Table 42, FIGS. 8A-8H), 40° C.±2° C. for up to 1 month (Table 43). Testing items including appearance, protein concentration, ELISA binding, pH, CE-SDS (reduced and non-reduced), SE-UPLC, and iCIEF.

TABLE 40
Study Parameters for IVX-03023 (−70 ° C. ± 10 ° C.)

| Test/ | Specification/ | | T = 1 M | T = 3 M | T=6 M |
|---|---|---|---|---|---|
| Time point | Acceptance Criteria | T = 0 | (2022 Dec. 09) | (2023 Feb. 09) | (2023-05-09) |
| Color | Colorless to slight yellow | Colorless | Colorless | Colorless | Colorless |
| Clarity | Clear to slightly opalescent | Clear | Clear | Clear | Clear |
| | solution | | | | |
| Protein | 10 ± 1 mg/mL | 10.0 | 10.0 | 10.0 | 10.0 |
| Concentration | | | | | |
| pH | 5.0 ± 0.5 | 5.1 | 5.1 | 5.0 | 5.1 |
| ELISA | 50-150% RP | 94.99 | NA | NA | NA |
| Binding | | | | | |
| CE-SDS | Heavy + Light | % Impurity = N/A | % Impurity = 3.77 | % Impurity = 4.4 | % Impurity = 4.2 |
| (reduced) | Chain Peak | % Light Chain = 23.3 | % Light Chain = 23.1 | % Light Chain = 23.7 | % Light Chain = 24.0 |
| | Area ≥90% | % Heavy Chain = 74.2 | % Heavy Chain = 73 | % Heavy Chain = 71.9 | % Heavy Chain = 71.8 |
| | | % Purity (% LC + | % Purity (% LC + | % Purity (% LC + | % Purity (% LC + |
| | | % HC) = 97.5 | % HC) = 96.1 | % HC) = 95.6 | % HC) = 95.8 |
| CE-SDS | Main Peak Purity ≥90% | % Pre-Peaks = 4.1 | % Pre-Peaks = 3.01 | % Pre-Peaks = 3.0 | % Pre-Peaks = 4.7 |
| (non-reduced) | | % Main Peak = 95.9 | % Main Peak = 96.99 | % Main Peak = 97.0 | % Main Peak = 95.3 |
| SEC-UPLC | Monomer: ≥90% | Monomer: 99.5% | Monomer: 99.45% | Monomer: 99.38% | Monomer: 99.43% |
| | HMW: Report Results | HMW: 0.6% | HMW: 0.55% | HMW: 0.58% | HMW: 0.53% |
| | LMW: Report Results | LMW: ND1 | LMW: ND1 | LMW: 0.03 | LMW: 0.04 |
| iCIEF | The difference in main | The difference in main | The difference in main | The difference in main | The difference in main |
| | peak pI value between | peak pI value between | peak pI value between | peak pI value between | peak pI value between |
| | sample and RS ≤0.2 | sample and RS = 0.0 | sample and RS = 0.1 | sample and RS = 0.0 | sample and RS = 0.0 |
| | Acidic Species: Report Result | Acidic Species: 38.4% | Acidic Species: 38.6% | Acidic Species: 38.7% | Acidic Species: 37.8% |
| | Main Species: Report Results | Main Species: 30.9% | Main Species: 31.8% | Main Species: 27.6% | Main Species: 31.1% |
| | Basic Species: Report Result | Basic Species: 30.7% | Basic Species: 29.6% | Basic Species: 33.7% | Basic Species: 31.1% |

TABLE 41
Study Parameters for IVX-03023 (5° C. ± 3° C.)

| Test/Time | Specification/ | | T = 1 M | T = 3 M |
|---|---|---|---|---|
| point | AcceptanceCriteria | T = 0 | (2022 Dec. 9) | (2023 Feb. 9) |
| Color | Colorless to | Colorless | Colorless | Colorless |
| | slight yellow | | | |
| Clarity | Clear to | Clear | Clear | Clear |
| | slightly opalescent | | | |
| | solution | | | |
| Visible | ≤10 Visible | Essentially free of | Essentially free of | Essentially free of |
| Particles | Proteinaceous | visible particles | visible particles | visible particles |
| | particles; No | | | |
| | foreign particles | | | |
| Subvisible | Report Results | ≥2 μm = 42 | ≥2 μm = 87 | ≥2 μm = 49 |
| Particles by | for ≥2 μm and | ≥5 μm = 18 | ≥5 μm = 22 | ≥5 μm = 22 |
| HIAC | ≥ 5 μm particles. | ≥10 μm = 12 | ≥10 μm = 7 | ≥10 μm = 10 |
| | ≥10 μm: | ≥25 μm = 1 | ≥25 μm = 0 | ≥25 μm = 0 |
| | ≤6000 particles/mL | | | |
| | ≥25 μm: | | | |
| | ≤600 particles/mL | | | |
| Protein | 10 ± 1 mg/mL | 10.2 | 10.0 | 10.1 |
| Concentration | | | | |
| pH | 5.0 ± 0.5 | 5.1 | 5.1 | 5.1 |
| ELISA Binding | 50-150% RP | 90.49 | NA | NA |
| CE-SDS | Heavy + Light Chain | % Impurity = N/A | % Impurity = 3.35 | % Impurity = 3.9 |
| (reduced) | Peak Area ≥ 90% | % Light Chain = 23.4 | % Light Chain = 23.3 | % Light Chain = 23.6 |
| | | % Heavy Chain = 74.1 | % Heavy Chain = 73.4 | % Heavy Chain = 72.5 |
| | | % Purity | % Purity | % Purity |
| | | (% LC + % HC) = 97.5 | (% LC + % HC) = 96.7 | (% LC+ % HC) = 96.1 |
| CE-SDS | Main Peak Purity ≥ 90% | % Pre-Peaks = 3.8 | % Pre-Peaks = 3.1 | % Pre-Peaks = 3.4 |
| (non-reduced) | | % Main Peak = 96.2 | % Main Peak = 96.9 | % Main Peak = 96.6 |
| SEC-UPLC | Monomer: ≥90% | Monomer: 99.5 | Monomer: 99.4 | Monomer: 99.2 |
| | HMW: Report | HMW: 0.5 | HMW: 0.6 | HMW: 0.8 |
| | Results (%) | LMW: ND¹ | LMW: ND¹ | LMW: 0.1 |
| | LMW: Report Results | | | |
| | (%) | | | |
| iCIEF | The difference in | The difference in | The difference in | The difference in |
| | main peak pI value | main peak pI value | main peak pI value | main peak pI value |
| | between sample and | between sample and | between sample and | between sample and |
| | RS ≤0.2 | RS = 0.0 | RS = 0.1 | RS = 0.0 |
| | Acidic Species: | Acidic Species: 38.9 | Acidic Species: 40.1 | Acidic Species: 38.5 |
| | Report Result (%) | Main Species: 29.3 | Main Species: 29.1 | Main Species: 28.3 |
| | Main Species: | Basic Species: 31.8 | Basic Species: 30.8 | Basic Species: 33.3 |
| | Report Results (%) | | | |
| | Basic Species: | | | |
| | Report Result (%) | | | |
| PS80 | 0.05 to 0.35% w/v | 0.016 | NA | NA |
| | | Test/Time | T = 6 M | T = 12 M |
| | | point | (2023 May 9) | (2023 Nov. 9) |
| | | Color | Colorless | Colorless |
| | | Clarity | Clear | Clear |
| | | Visible | Essentially free of | Essentially free |
| | | Particles | visible particles | of visible particles |
| | | Subvisible | ≥2 μm = 100 | ≥2 μm = 144 |
| | | Particles by | ≥ 5 μm = 32 | ≥5 μm = 37 |
| | | HIAC | ≥10 μm = 12 | ≥10 μm = 10 |
| | | | ≥25 μm = 0 | ≥25 μm = 0 |
| | | Protein | 10.0 | 10.0 |
| | | Concentration | | |
| | | pH | 5.1 | 5.0 |
| | | ELISA Binding | NA | 100.80 |
| | | CE-SDS | % Impurity = 4.7 | % Impurity = 4.5 |
| | | (reduced) | % Light Chain = 23.9 | % Light Chain = 28.7 |
| | | | % Heavy Chain = 71.4 | % Heavy Chain = 66.8 |
| | | | % Purity | % Purity |
| | | | (% LC + % HC) = 95.3 | (% LC + % HC) = 95.5 |
| | | CE-SDS | % Pre-Peaks = 4.5 | % Pre-Peaks = 4.6 |
| | | (non-reduced) | % Main Peak = 95.5 | % Main Peak = 95.4 |
| | | SEC-UPLC | Monomer: 99.1 | Monomer: 98.9 |
| | | | HMW: 0.8 | HMW: 0.9 |
| | | | LMW: 0.1 | LMW: 0.1 |
| | | iCIEF | The difference in | The difference in |
| | | | main peak pI value | main peak pI value |
| | | | between sample and | between sample and |
| | | | RS = 0.0 | RS = 0.0 |
| | | | Acidic Species: 36.8 | Acidic Species: 34.1 |
| | | | Main Species: 30.1 | Main Species: 25.5 |
| | | | Basic Species: 33.1 | Basic Species: 40.5 |
| | | PS80 | 0.020 | 0.018 |

TABLE 42
Study Parameters for IVX-06076 (25° C. ± 2° C.)

| Test/ | Specification/ | | T = 1 M | T = 3 M | T = 6 M |
|---|---|---|---|---|---|
| Time point | Acceptance Criteria | T = 0 | (2022 Dec. 09) | (2023 Feb. 09) | (2023 May 09) |
| Color | Colorless to slight yellow | Colorless | Colorless | Colorless | Colorless |
| Clarity | Clear to slightly opalescent | Clear | Clear | Clear | Clear |
| | solution | | | | |
| Visible | ≤10 Visible | Essentially free of | Essentially free of | Essentially free of | Essentially free of |
| Particles | Proteinaceous particles; | visible particles | visible particles | visible particles | visible particles |
| | No foreign particles | | | | |
| Subvisible | Report Results for ≥2 μm | ≥2 μm = 42 | ≥2 μm = 22 | ≥2 μm = 67 | ≥2 μm = 149 |
| Particles by | and ≥5 μm particles. | ≥5 μm = 18 | ≥5 μm = 17 | ≥5 μm = 27 | ≥5 μm = 29 |
| HIAC | ≥10 μm: ≤6000 particles/mL | ≥10 μm = 12 | ≥10 μm = 9 | ≥10 μm = 5 | ≥10 μm = 10 |
| | ≥25 μm: ≤600 particles/mL | ≥25 μm = 1 | ≥25 μm = 2 | ≥25 μm = 2 | ≥25 μm = 0 |
| Protein | 10 ± 1 mg/mL | 10.2 | 10.0 | 10.1 | 10.0 |
| Concentration | | | | | |
| pH | 5.0 ± 0.5 | 5.1 | 5.1 | 5.1 | 5.1 |
| ELISA | 50-150% RP | 90.49 | NA | NA | 66.95 |
| Binding | | | | | |
| CE-SDS | Heavy + Light | % Impurity = N/A | % Impurity = 3.98 | % Impurity = 5.0 | % Impurity = 11.8 |
| (reduced) | Chain Peak | % Light Chain = 23.4 | % Light Chain = 23.4 | % Light Chain = 23.9 | % Light Chain = 23.8 |
| | Area ≥90% | % Heavy Chain = 74.1 | % Heavy Chain = 72.6 | % Heavy Chain = 71.2 | % Heavy Chain = 64.4 |
| | | % Purity (% LC + | % Purity (% LC + | % Purity (% LC + | % Purity(% LC + |
| | | % HC) = 97.5 | % HC) = 96 | % HC) = 95.0 | % HC) = 88.2 |
| CE-SDS | Main Peak Purity ≥90% | % Pre-Peaks = 3.8 | % Pre-Peaks = 3.4 | % Pre-Peaks = 4.0 | % Pre-Peaks |
| (non-reduced) | | % Main Peak = 96.2 | % Main Peak = 96.61 | % Main Peak = 96.0 | (LMWs) = 6.9 |
| | | | | | HMWs = 1.5 |
| | | | | | % Main Peak = 91.5 |
| SEC-UPLC | Monomer: ≥90% | Monomer: 99.5 | Monomer: 99.01 | Monomer: 98.01 | Monomer: 91.90 |
| | HMW: Report Results (%) | HMW: 0.5 | HMW: 0.83 | HMW: 1.53 | HMW: 4.92 |
| | LMW: Report Results (%) | LMW: ND¹ | LMW: 0.16 | LMW: 0.40 | LMW: 3.18 |
| iCIEF | The difference in main | Basic Species: 31.8 | The difference in main | The difference in main | The difference in main |
| | peak pI value between | The difference in main | peak pI value between | peak pI value between | peak pI value between |
| | sample and RS ≤0.2 | peak pI value between | sample and RS = 0.1 | sample and RS = 0.0 | sample and RS = 0.0 |
| | Acidic Species: Report | sample and RS = 0.0 | Acidic Species: 36.8 | Acidic Species: 36.4 | Acidic Species: 52.1 |
| | Result (%) | Acidic Species: 38.9 | Main Species: 29.2 | Main Species: 19.6 | Main Species: 12.1 |
| | Main Species: Report | Main Species: 29.3 | Basic Species: 34 | Basic Species: 44.0 | Basic Species: 35.8 |
| | Results (%) | | | | |
| | Basic Species: Report | | | | |
| | Result (%) | | | | |
| PS80 | 0.05 to 0.35% w/v | 0.016 | NA | NA | NA |

TABLE 43
Study Parameters for IVX-06076 (40° C. ± 2° C.)

| Test/ | Specification/ | | T = 2 W | T = 1 M |
|---|---|---|---|---|
| Time point | Acceptance Criteria | T = 0 | (2022 Nov. 23) | (2023-12-09) |
| Color | Colorless to slight yellow | Colorless | Colorless | Colorless |
| Clarity | Clear to slightly opalescent | Clear | Clear | Clear |
| | solution | | | |
| Visible | ≤10 Visible | Essentially free of | Essentially free of | Essentially free of |
| Particles | Proteinaceous particles; | visible particles | visible particles | visible particles |
| | No foreign particles | | | |
| Subvisible | Report Results for ≥2 μm | ≥2 μm = 42 | ≥2 μm = 155 | ≥2 μm = 45 |
| Particles | and ≥5 μm particles. | ≥5 μm = 18 | ≥5 μm = 60 | ≥5 μm = 20 |
| by HIAC | ≥10 μm: ≤6000 particles/mL | ≥10 μm = 12 | ≥10 μm = 15 | ≥10 μm = 9 |
| | ≥25 μm: ≤600 particles/mL | ≥25 μm = 1 | ≥25 μm = 2 | ≥25 μm = 0 |
| Protein | 10 ± 1 mg/mL | 10.2 | 10.1 | 10.1 |
| Concentration | | | | |
| pH | 5.0 ± 0.5 | 5.1 | 5.1 | 5.1 |
| ELISA | 50-150% RP | 90.49 | NA | 86.14 |
| Binding | | | | |
| CE-SDS | Heavy + Light | % Impurity = N/A | % Impurity = 4.6 | % Impurity = 4.71 |
| (reduced) | Chain Peak | % Light Chain = 23.4 | % Light Chain = 23.6 | % Light Chain = 23.5 |
| | Area ≥90% | % Heavy Chain = 74.1 | % Heavy Chain = 71.8 | % Heavy Chain = 71.8 |
| | | % Purity (% LC + | % Purity (% LC + | % Purity (% LC + |
| | | % HC) = 97.5 | % HC) = 95.4 | % HC) = 95.3 |
| CE-SDS | Main Peak Purity ≥90% | % Pre-Peaks = 3.8 | % Pre-Peaks = 3.91 | % Pre-Peaks = 4.67 |
| (non-reduced) | | % Main Peak = 96.2 | % Main Peak = 96.08 | % Main Peak = 95.32 |
| SEC-UPLC | Monomer: ≥90% | Monomer: 99.5 | Monomer: 97.7 | Monomer: 95.32 |
| | HMW: Report Results (%) | HMW: 0.5 | HMW: 1.89 | HMW: 3.9 |
| | LMW: Report Results (%) | LMW: ND¹ | LMW: 0.41 | LMW: 0.78 |
| iCIEF | The difference in main | The difference in main | The difference in main | The difference in main |
| | peak pI value between | peak pI value between | peak pI value between | peak pI value between |
| | sample and RS ≤0.2 | sample and RS = 0.0 | sample and RS = 0.0 | sample and RS = 0.0 |
| | Acidic Species: Report | Acidic Species: 38.9 | Acidic Species: 36.8 | Acidic Species: 33.4 |
| | Result (%) | Main Species: 29.3 | Main Species: 25.9 | Main Species: 25.2 |
| | Main Species: Report | Basic Species: 31.8 | Basic Species: 37.3 | Basic Species: 41.4 |
| | Results (%) | | | |
| | Basic Species: Report | | | |
| | Result (%) | | | |
| PS80 | 0.05 to 0.35% w/v | 0.016 | NA | NA |

IVX-06076 Drug substance was stable at recommended long term storage temperature of −70±−10° C. for 6 months (Table 40, FIGS. 6A-6D) and well above the acceptance criteria for all the product quality attributes. Drug product was stable at recommended long term storage temperature of 2-8° C. for 1 year (Table 41, FIGS. 7A-7H) and accelerated storage temperature of 25° C. for 6 months (Table 42, FIGS. 8A-8H) well above the acceptance criteria for all the product quality attributes. Potency of the molecule is maintained. pH, visible particles and osmolarity results all show no meaningful differences over time (data not shown in the graphs)

The complete disclosure of all patents, patent applications, and publications, and electronically available material (including, for instance, nucleotide sequence submissions in, e.g., GenBank and RefSeq, and amino acid sequence submissions in, for example, SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A stable pharmaceutical formulation comprising:
(a) 5 to 100 mg/ml of an antibody comprising a polypeptide having a canine or feline CDR of variable antibody domain, CH2, CH3, IgG Fc region variant, or a canine or feline FcRn-binding region thereof;
(b) 4.0 to 50 mM of a buffering agent at a pH in the range of from 4.5 to 7.0;
(c) 0.01 to 35% (w/v) of a tonicity and/or a stabilizing agent; and
(d) 0.01 to 10% (w/v) of a surfactant;
wherein the formulation is a single-dose or multi-dose formulation, and
wherein the formulation retains stability for up to twenty-four months in solution.

2. The pharmaceutical formulation of claim 1 further comprising 0.01 to 5.0 mM of a chelating agent.

3. The pharmaceutical formulation of claim 1, wherein the multi-dose formulation further comprises an antimicrobial preservative selected from a group comprising benzyl alcohol, phenol, m-cresol, benzalkonium chloride, benzalthonium chloride, phenoxyethanol and methyl paraben or mixtures thereof at about a concentration of 0.1 to 1.5% (w.v).

4. The pharmaceutical formulation of claim 1, wherein the buffering agent is selected from a group consisting of sodium acetate, histidine, histidine hydrochloride (HCl), succinate, phosphate, Tris, diethanolamine, citrate, acetate, other organic acids and mixtures thereof.

5. The pharmaceutical formulation of claim 1, wherein the buffering agent is present at a concentration of 5 to 30 mM and maintains a physiologically suitable pH in the range of from pH 5 to pH 6.5.

6. The pharmaceutical formulation of claim 1, wherein the tonicity and/or stabilizing agent is selected from a group consisting of CaCl₂), NaCl, MgCl₂, lactose, sorbitol, sucrose, mannitol, trehalose, raffinose, polyethylene glycol, hydroxyethyl starch, glycine and mixtures thereof.

7. The pharmaceutical formulation of claim 6, wherein the tonicity and/or stabilizing agent is preferably sucrose, trehalose, sorbitol.

8. The pharmaceutical formulation of claim 1, wherein the tonicity and/or stabilizing and/or stabilizing agent is present at a concentration of 0.02% to 10% (w.v).

9. The pharmaceutical formulation of claim 1, wherein the chelating agent is selected from a group consisting of aminopolycarboxylic acids, hydroxyaminocarboxylic acids, N-substituted glycines, 2-(2-amino-2-oxoethyl) aminoethane sulfonic acid (BES), deferoxamine (DEF), citric acid, niacinamide, desoxycholates, diethylenetriamine pentaacetic acid 5 (DTPA), nitrilotriacetic acid (NTA), N-2-acetamido-2-iminodiacetic acid (ADA), bis(aminoethyl)glycolether, N,N,N′,N′-tetraacetic acid (EGTA), trans-diaminocyclohexane tetraacetic acid (DCTA), glutamic acid, and aspartic acid, N-hydroxyethyliminodiacetic acid (HIMDA), N,N-bis-hydroxyethylglycine (bicine) and N-(trishydroxymethylmethyl) 10 glycine (tricine), glycylglycine, sodium desoxycholate, ethylenediamine, propylenediamine, diethylenetriamine, triethylenetetraamine (trien), ethylenediaminetetraacetic acid (EDTA), disodium EDTA, calcium EDTA oxalic acid, malate, citric acid, citric acid monohydrate, and trisodium citrate-dihydrate, 8-hydroxyquinolate, amino acids, histidine, cysteine, methionine, peptides, polypeptides, and proteins and mixtures thereof.

10. The pharmaceutical formulation of claim 9, wherein the chelating agent is preferably EDTA, disodium EDTA, calcium EDTA.

11. The pharmaceutical formulation of claim 9, wherein the chelating agent is present at a concentration of 0.01 to 5 mM.

12. The pharmaceutical formulation of claim 1, wherein the surfactant is selected from a group consisting of polysorbates, poloxamers, tritons, sodium dodecyl sulfate, sodium laurel sulfate, sodium octyl glycoside, lauryl-sulfobetaine, myristyl-sulfobetaine, linoleyl-sulfobetaine, stearyl-sulfobetaine, lauryl-sarcosine, myristyl-sarcosine, linoleyl-sarcosine, stearyl-sarcosine, linoleyl-betaine, myristyl-betaine, cetyl-betaine, lauroamidopropyl-betaine, cocamidopropyl-betaine, linoleamidopropyl-betaine, myristamidopropyl-betaine, palmidopropyl-betaine, isostearamidopropyl-betaine, myristamidopropyl-dimethylamine, palmidopropyl-dimethylamine, isostearamidopropyl-dimethylamine, sodium methyl cocoyl-taurate, disodium methyl oleyl-taurate, dihydroxypropyl PEG 5 linoleammonium chloride, polyethylene glycol, polypropylene glycol, and mixtures thereof.

13. The pharmaceutical formulation of claim 12, wherein the polysorbate is selected from the group consisting of polysorbate 20, polysorbate 21, polysorbate 40, polysorbate 60, polysorbate 61, polysorbate 65, polysorbate 80, polysorbate 81, polysorbate 85, and mixtures thereof.

14. The pharmaceutical formulation of claim 1, wherein the surfactant is present at a concentration of 0.002 to 0.5% (w/v).

15. The pharmaceutical formulation of claim 1, further comprising an antioxidant selected from a group consisting of GLA (gamma-linolenic acid)-lipoic acid, DHA (docosahexaenoic acid)-lipoic acid, GLA-tocopherol, di-GLA-3,3′-thiodipropionic acid, DGLA (dihomo-gamma-linolenic acid), AA (arachidonic acid), SA (salicylic acid), EPA (eicosapentaenoic acid) or DHA (docosahexaenoic acid), phenolic anti-oxidants including, polyenes, unsaturated sterols, ascorbic acid, organosulfur compounds, terpenes and amino acid antioxidants.

16. The pharmaceutical formulation of claim 15, wherein the amino acid antioxidants are selected from a group consisting of methionine, cysteine, carnosine and analogs thereof.

17. The pharmaceutical formulation of claim 15, wherein the antioxidant is present at a concentration of 0.01 mM to about 50 mM.

18. The pharmaceutical formulation of claim 1, further comprising a preservative present at a concentration of 0.001% w/v to 10% w/v wherein the preservative is selected from a group consisting of phenols, m-cresol, benzyl alcohol, benzalkonium chloride, benzalthonium chloride, phenoxyethanol and methyl paraben.

19. The pharmaceutical formulation of claim 1, wherein the formulation is suitable for oral, rectal, transmucosal, intestinal, or parenteral administration.

20. The pharmaceutical formulation of claim 19, wherein parenteral administration is selected from intravenous, intraarterial, intraperitoneal, intrathecal, intraventricular, intraurethral, intrasternal, intracranial, intramuscular, intra-ossial, intradermal or subcutaneous administration.
